(19) United States
(12) Patent Application Publication
(10) Pub. No.: US 2024/0173272 A1
(43) Pub. Date: May 30, 2024

(54) EXTENDED-RELEASE LIQUID COMPOSITIONS OF MEXILETINE FOR ORAL ADMINISTRATION

(71) Applicant: LUPIN ATLANTIS HOLDINGS SA, Zug (CH)

(72) Inventors: Sajeev Chandran, Pune (IN); Hemant Bhaurao Banbale, Pune (IN); Dipak Dattatray Joshi, Aurangabad (IN); Shailesh Bhamare, Bengaluru (IN); Makarand Krishnakumar Avachat, Pune (IN)

(21) Appl. No.: 18/534,710

(22) Filed: Dec. 10, 2023

Related U.S. Application Data
(63) Continuation-in-part of application No. PCT/EP2022/065917, filed on Jun. 10, 2022.

(30) Foreign Application Priority Data
Jun. 11, 2021 (IN) 202131026198

(51) Int. Cl. A61K 31/138; A61K 9/00

(52) U.S. Cl. CPC A61K 31/138; A61K 9/0053

(57) ABSTRACT

The present invention relates to controlled-release pharmaceutical compositions of mexiletine compounds in combination with a release retardant agent, e.g., with an ion exchange resin, and the method of making and using such compositions. In a particular aspect, the invention relates to a controlled release suspension of mexiletine, or a pharmaceutically acceptable salt thereof complexed with an effective amount of a pharmaceutically acceptable ion exchange resin. In particular, the invention provides a controlled-release suspension of mexiletine to be administered once a day for the treatment of myotonic disorders. Further, the present invention provides new mexiletine compound-containing compositions having improved characteristics in terms of bioavailability, dosing frequency, and patient compliance.

### RELATED APPLICATIONS AND PRIORITY

This application is a continuation-in-part of PCT/EP2022/065917, filed on Jun. 10, 2022, which claims priority to Indian Patent Application No. 202131026198, filed on Jun. 11, 2021. The Applicant hereby incorporates the entirety of these above-referenced priority applications by reference.

### FIELD OF THE INVENTION

The present invention relates to oral controlled release pharmaceutical compositions of mexiletine or pharmaceutically acceptable salt thereof suitable for oral administration. The present invention also relates to a process of preparing and use of said compositions for treating myotonic disorders.

### BACKGROUND OF THE INVENTION

Myotonia refers to a neuromuscular condition in which the relaxation of a muscle is impaired. It can be mild or severe, interfering with daily activities such as walking, climbing stairs, or opening and closing the eyelids. Myotonia is caused by an abnormality in the muscle membrane and is often associated with inherited neurological disorders. It is commonly seen in individuals with myotonic muscular dystrophy, myotonia congenita, and in people who have one of a group of neurological disorders called channelopathies. It may be acquired or inherited and is caused specifically by mutations in the chloride, sodium, or potassium ion transport channels in the muscle membrane. Moreover, affected individuals have proximal muscle weakness, and it spreads to eventually involve all muscle groups, including oropharyngeal muscles, and hence swallowing impairment is prevalent in patients with myotonia.

Mexiletine hydrochloride is a class 1b antiarrhythmic medication with a high affinity for muscle sodium channels and is still used to treat arrhythmias. One commercial mexiletine hydrochloride product, sold under the trademark MEXITIL® by Boehringer Ingelheim Pharmaceuticals, Inc., is available in 150 mg, 200 mg, and 250 mg capsules administered thrice daily.

The structure of mexiletine hydrochloride is shown below:

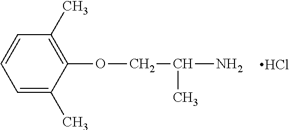

Mexiletine is rapidly and completely absorbed following oral administration with a bioavailability of about 90%. Labbé L, Turgeon J. Clinical pharmacokinetics of mexiletine. Clin Pharmacokinet. 1999 November; 37(5):361-84. doi: 10.2165/00003088-199937050-00002.

Peak plasma concentrations following oral administration occur within 1 to 4 hours, and a linear relationship between dose and plasma concentration is observed in the dose range of 100 to 600 mg. Id. Mexiletine is eliminated slowly in humans (with an elimination half-life of 10 hours). Id. Mexiletine is a weak base (pKa of about 9.1) and is absorbed in the intestine with absorption in the stomach reportedly being negligible. European Medicines Agency, Assessment Report: Namuscla (Oct. 18, 2018) (EMA/831802/2018) (“EMA 2018”). Reportedly, food has no effect on the rate of absorption of previously described mexiletine products. Id. at p. 38.

In recent years, mexiletine has found increasing utility in the treatment of myotonia. For example, the mexiletine hydrochloride immediate-release capsule NaMuscla® by Lupin Limited is used for the treatment of symptomatic myotonia in adults with non-dystrophic myotonic disorders. The approved maintenance dose is between 167 mg and 500 mg, i.e., 1 to 3 capsules administered at 8 hourly intervals. Nearly all studies in support of NaMuscla's European approval involved three-times-daily (TID) administration (EMA, 2018).

Generally, drug therapies that use immediate-release oral dosage forms need to be administered at spaced intervals to maintain a desired therapeutic effect, and hence, patients suffering from myotonia or similar conditions often have trouble complying with this administration schedule. As in the case of myotonia patients, muscles of the face, mouth, and throat are affected, which causes problems with swallowing, especially while using high doses and frequent administration of immediate-release dosage forms. Further, mexiletine acts by blocking fast sodium channels, hence the high peak trough plasma level of mexiletine from multiple dosing regimens of immediate release composition is associated with the cardiac events. Additionally, when administered as immediate release composition, the propensity of such formulations to dump the entire dose in the early portion of the GI tract tends to cause other GI-related side effects like emesis.

The desire to extend the duration of the therapeutic effect of active pharmaceutical ingredients often leads to the proposed or actual development of controlled drug release formulations, which are often associated with reduced frequency of administration. Various techniques for developing such formulations are known. For example, ion exchange resins have been an approach utilized for achieving controlled release dosage forms, and various attempts have been made to further utilize the technology in powders, liquids, suspensions, and dispersible tablet compositions as well. Ion exchange resins are cross-linked water-insoluble polymer-carrying, ionizable functional groups and have received considerable attention from pharmaceutical scientists because of their versatile properties as a drug delivery vehicle. Not all drugs are suitable for ion exchange resins. For example, active pharmaceutical ingredients with a biological half-life of over 8 hours, such as mexiletine, are considered generally unsuitable for such formulations (see, e.g., Dp, Venkatesh & Karki, Roopa & Goli, Divakar & Jha, Sajal. (2013). JOURNAL OF PHARMACY AND PHARMACEUTICAL SCIENCES. Vol. 2, Issue 6, pp. 4764-77). There are other factors that might suggest mexiletine to be an unsuitable candidate for such approaches. Id.

Other types of sustained-release formulation concepts have been proposed for application to mexiletine products in the art. For example, CN101032462 discloses sustained release preparation of mexiletine hydrochloride having a dual release profile, i.e., immediate release and sustained release, requires twice-daily administration for the treatment of arrhythmia. U.S. Pat. No. 4,459,279 discloses a process of making mexiletine immediate-release mini tablets by admixing the mexiletine with conventional excipients such as lactose, polyvinyl pyrrolidone, and magnesium stearate. Further, the tablets are coated with a release retardant spray solution to produce sustained-release mini tablets. However, the disclosure of the above-mentioned references appears to fail to disclose a suitable once-a-day controlled-release composition of mexiletine that is suitable for the treatment of myotonia. For example, when administered in high doses or once daily, the formulations disclosed in the above-mentioned references are expected to be unsuitable because they become too bulky and hence very difficult to swallow specifically in patients with myotonia. Dose dumping is also a known problem associated with extended-release formulations that might make such formulations unsuitable for use in connection with mexiletine products for the treatment of conditions such as myotonia. Indeed, despite the known usefulness of mexiletine for the treatment of myotonia, and the existence of such proposed formulations in the art, mexiletine formulations that offer these or other significant advantages to myotonia patients appear to be both lacking from the market and active development. Such facts evidence that developing improved mexiletine formulations having advantageous properties for patients suffering from myotonia or similar conditions requires inventive ingenuity.

### Construction, Terms, and Acronyms

This section offers guidelines for reading this disclosure. The intended audience for this disclosure (“readers”) are people having ordinary skills in the practice of technologies discussed or used herein. Readers may also be called “skilled persons,” and such technologies are called “the art.” Terms such as “understood,” “known,” and “ordinary meaning,” refer to the general knowledge of skilled persons.

The following description of certain terms and acronyms is provided to assist readers in understanding the invention. Additional acronyms may be only provided in other parts of this disclosure, and acronyms that are well-known in the art may not be provided here.

The term “uncontradicted” means not contradicted by this disclosure or by logic, or plausibility, or unsuitability based on knowledge of skilled persons.

Disclosed here are several different but related exemplary aspects of the invention (referred also to as, e.g., “cases,” “facets,” or “embodiments”). The invention encompasses all aspects as described individually and as can be arrived at by any combination of such individual aspects. The breadth and scope of the invention should not be limited by any exemplary embodiment(s). No language in this disclosure should be construed as indicating any element/step is essential to the practice of the invention unless such a requirement is explicitly stated. Uncontradicted, any aspect(s) described in any part of this disclosure can be combined with any other aspect(s) in any other part.

Uncontradicted, all technical/scientific terms used here generally have the same meanings as commonly understood by skilled persons, regardless of any narrower examples or descriptions provided here (including any term introduced initially in quotations). However, aspects characterized by the inclusion of elements, steps, etc., associated with specific descriptions provided here are distinct embodiments of the invention. Uncontradicted, disclosure of any aspect using known terms, which terms are narrowed by example or otherwise in this disclosure, implicitly discloses related aspects in which such terms are alternatively interpreted using the broadest reasonable interpretation of skilled persons.

Uncontradicted, “or” means “and/or” here, regardless of any occasional inclusion of “and/or” (e.g., phrases such as “A, B, or C” and “A, B, and/or C” simultaneously disclose aspects including (1) all of A, B, and C; (2) A and C; (3) A and B; (4) B and C; (5) only A; (6) only B; and (7) only C (and also support sub-groupings, such as “A or B,” “A or C,” etc.)).

Uncontradicted, “also” means “also or alternatively.” Uncontradicted, “here” & “herein” mean “in this disclosure.” The term “i.a.” means “inter alia” or “among other things.” “Also known as” is abbreviated “aka” or “AKA.” (and can mean is otherwise referred to, even if the relationship between the terms is not well known). “Elsewhere” means “elsewhere herein.”

For conciseness, symbols are used where appropriate. E.g., “&” is used for “and,” & “˜” for “about.” Symbols such as < and > are given their ordinary meaning (e.g., “≤” means “less than or equal to” & “≥” means “greater than or equal to”). A slash “/” between terms here can represent “or” (“A/B” means “A or B”) or identify synonyms of an element, as will be clear from context. The inclusion of “(s)” after an element or a step indicates that ≥1 of such an element is present, step performed, and the like. E.g., “element(s)” means both 1 element or ≥2 elements, with the understanding that each thereof is an independent aspect of the invention. Uncontradicted, any aspect disclosed herein with an element or step expressed in the singular provides implicit support for a corresponding embodiment in which the element(s)/step(s) are present in the plural (two or more), and vice versa.

Use of the abbreviation “etc.” (or “et cetera”) in association with a list of elements/steps means any or all suitable combinations of the recited elements/steps or any known equivalents of such recited elements/steps for achieving the function(s) of such elements/steps known in the art. Readers should interpret phrases like “and the like” similarly.

Uncontradicted, terms such as “and combinations,” “or combinations,” and “combinations thereof,” etc., regarding listed elements/steps, means any or all possible/suitable combinations of the associated elements/steps. Thus, e.g., uncontradicted, a phrase like “combination of any thereof” refers to any or all combinations.

Aspects may be described as suitable for use(s) disclosed herein. Uncontradicted, terms such as “suitability” mean acceptable or appropriate for performing a particular function/achieving particular state(s)/outcome(s), and typically means effective, practical, and non-deleterious/harmful in the context the term is used. E.g., uncontradicted, the term “suitable” means appropriate, acceptable, or in contexts sufficient, or providing at least generally or substantially all of an intended function, without causing or imparting significant negative/detrimental impact. Uncontradicted terms such as “suitable” or “suitable for” also mean that the associated components, methods, steps, devices, etc., are generally safe and effective, e.g., have sufficient strength/potency, quality, or safety for their intended application. Such elements may be judged by relevant experts, consumer approval/surveys, or regulatory authority review, or as may be established by e.g., scientific measures, commercial success, expert opinions (e.g., medical opinions), or, in aspects, controlled and adequate clinical studies, adequately powered preclinical or consumer testing, and the like. Components, elements, and steps described as “suitable” should be interpreted in a similar manner. Uncontradicted, any object, step, or other element of any aspect is suitable for the purpose(s)/function(s) or characteristic(s) associated with such element herein or in the art, even if the word suitable is not used to characterize such an object, step, element, etc.

Uncontradicted, heading(s) (e.g., “Construction, Terms . . . ”) and subheadings are included for convenience and do not limit the scope of any aspect(s). Uncontradicted aspect(s), step(s), or element(s) described under one heading can apply to any other aspect(s), even those described in different sections, headings, etc.

Ranges of values are used to represent each value falling within such range that is within an order of magnitude of the smallest endpoint of the range without having to explicitly write each value of the range. E.g., a recited range of 1-2 implicitly discloses each of 1.0, 1.1, 1.2, . . . 1.9, and 2.0, and 10-100 implicitly discloses each of 10, 11, 12, . . . 98, 99, and 100. Uncontradicted, all ranges include the range's endpoints, regardless of how a range is described. E.g., “between 1-5” includes 1 and 5 in addition to 2, 3, and 4 (and all numbers between such numbers within an order of magnitude of such endpoints, e.g., 1.0, 1.1, . . . 4.9, and 5.0). For the avoidance of doubt, any number within a range, regardless of the order of magnitude of the number, is covered by the range (e.g., a range of 2-20 covers 18.593). Uncontradicted, readers will understand that any two values in a range provided as a list herein can be combined as endpoints to form a range defining a more particular aspect of the invention (e.g., if a list of values 1, 2, 3, 4, and 5 of element X is provided, readers will understand that the disclosure implicitly discloses an aspect comprising 2-4 X, 3-5 X, and 1-3 X, etc.

Terms of approximation (e.g., “about,” “˜” or “approximately”) are used (1) to refer to a set of related values or (2) where a precise value is difficult to define (e.g., due to limits of measurement). Uncontradicted, all exact values provided here simultaneously/implicitly disclose corresponding approximate values and vice versa (e.g., disclosure of “about 10” provides explicit support for the use of 10 exactly in such aspect/description). Ranges described with approximate value(s) include all values encompassed by each approximate endpoint, regardless of presentation (e.g., “about 10-20” has the same meaning as “about 10-about 20”). The scope of value(s) encompassed by an approximate term typically depends on the context of the disclosure, criticality or operability, statistical significance, understanding in the art, etc. In the absence of guidance here or in the art for an element, terms such as “about” when used in connection with an element should be interpreted as +/−10% of the indicated value(s) and implicitly disclosing +/−5%, +/−2%, +/−1%, and +/−0.5%.

This disclosure includes aspects associated with particular characteristics, such as amounts of components (or ranges thereof), In cases, several such characteristics of varying scope may be provided. Readers will understand that each such characteristic can be associated with particular properties that distinguish such aspects from other aspects, and, accordingly, each such range can be viewed as critical to a particular aspect of the invention, even if the associated results, properties, functions, etc., associated with such aspects are not directly communicated in association with such characteristics.

Lists of aspects, elements, steps, and features are sometimes employed for conciseness. Unless indicated, each member of each list should be viewed as an independent aspect. Each aspect defined by any individual member of a list can have and often will have, nonobvious properties vis-a-vis aspects characterized by other members of the list.

Uncontradicted, the terms “a” and “an” and “the” and similar referents encompass both the singular and the plural form of the referenced element, step, or aspect. Uncontradicted, terms in the singular implicitly convey the plural and vice versa herein (in other words, disclosure of an element/step implicitly discloses the corresponding use of such/similar elements/steps and vice versa). Hence, e.g., a passage regarding an aspect including X step supports a corresponding aspect including several X steps. Uncontradicted, any mixed use of a referent such as “a” in respect of one element/step or characteristic and “one or more of” with respect to another element/step or characteristic in a paragraph, sentence, aspect, or claim, does not change the meaning of such referents. Thus, for example, if a paragraph describes a composition comprising “an X” and “one or more Ys,” the paragraph should be understood as providing disclosure of “one or more Xs” and “one or more Ys.”

“Significant” and “significantly” mean results/characteristics that are statistically significant using ≥1 appropriate test(s)/trial(s) in the given context (e.g., p<0.05/0.01). “Detectable” means measurably present/different using known detection tools/techniques. The acronym “DOS” (or “DoS”) means “detectable(ly) or significant(ly).” The term “measurably” means at a measurable level. The term detectable provides implicit disclosure for aspects that are “measurable” and the term “measurable” implicitly supports aspects where the measured or measurable element is “detectable.”

Uncontradicted, for any value here that is not accompanied by a unit of measurement (e.g., a weight of 50 or a length of 20), any previously provided unit for the same element/step or the same type of element/step will apply, or, in cases where no such disclosure exists, the unit most commonly used in association with such an element/step in the art will apply or, in cases where no such disclosure exists, the unit most commonly used in association with such an element/step in the art will apply (e.g., a ruler with a length of 12 would be recognized as being 12 inches long).

Uncontradicted, the terms “including,” “containing,” “comprising,” and “having” mean “including, but not limited to” or “including, without limitation.” Uncontradicted, use of terms such as comprising and including regarding elements/steps means including any detectable number or amount of an element or including any detectable performance of a step/number of steps (with or without other elements/steps).

For conciseness, description of an aspect “comprising” or “including” an element, with respect to a collection/whole (e.g., a system, device, or composition), implicitly provides support for any detectable amount/number or ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the whole/collection being made up of the element, or essentially all of the whole/collection being made up of the element (i.e., that the collection consists essentially of the referenced element). Similarly, a method described as including a step with respect to an effect/outcome implicitly provides support for the referenced step providing ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the effect/outcome, representing ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the steps/effort performed, or both. Explicit listing of percentages of elements/steps in connection with aspects does not limit or contradict such implicit disclosure. Uncontradicted, any disclosure of an object/element (e.g., composition, device, or system) “comprising” or “including” element(s) provides implicit support for an alternative corresponding aspect that is characterized by the object consisting of that element or “consisting essentially of” that element (excluding anything that would “materially affect” the “basic and novel characteristic(s)” of the invention. Uncontradicted, any specific uses of phrases such as “consists,” “consists of,” and “consisting essentially of” herein does not undermine this principle of construction. Readers will understand the “basic and novel characteristic(s)” of the invention, and the scope of what constitutes a “material affect” (or “material effect”) of such “basic and novel characteristics” will vary with the specific applicable aspect. In aspects, the basic and novel characteristics include one or more intended functions and levels of performance. In one aspect, the basic and novel characteristics include suitability, effectiveness, or both. The basic and novel characteristics of any aspect of the invention also include, of course, any specific recited and associated elements of an aspect. In an aspect, a material effect is an effect that reduces, diminishes, eliminates, counteracts, cancels, or prevents one or more of such functions in one or more respects (e.g., delaying onset, reducing scope, reducing duration, reducing output, reducing the level of applicability, reducing effect, or combinations thereof). In an aspect, a material effect is one that changes such functions by making such functions impractical, difficult to obtain, or materially more expensive or otherwise costly in terms of inputs. From this and the other guidance provided herein, readers can understand the scope of an aspect that is defined by consisting essentially of a collection of elements. E.g., a composition that consists essentially of elements A and B, which are helpful towards human health, would exclude element C, which is known to reduce the efficacy of A, and also exclude element D, which is known to be toxic.”

Uncontradicted terms such as “comprising” when used in connection with a step of a method, provide implicit support for performing the step once, ≥2 times, or one or more times until an associated function/effect is achieved.

Uncontradicted, the term “one” means a single type, single iteration/copy/thing, of a recited element or step, or both, which will be clear from the context. For example, the referent “one” used with a component of a composition can refer to one type of element (which may be present in numerous copies, as in the case of an ingredient in a composition), one unit of the element, or both. Similarly, “one” component, a “single” component, or the “only component” of a system typically means 1 type of element (which may be present in numerous copies), 1 instance/unit of the element, or both. Further, “one” step of a method typically means performing one type of action (step), one iteration of a step, or both. Uncontradicted, a disclosure of “one” element provides support for both, but uncontradicted, any claim to any “one” element means one type of such an element (e.g., a component of a composition/system).

Uncontradicted, the term “some” means ≥2 copies/instances or ≥5% of a listed collection/whole is or is made up of an element. Regarding methods, some means ≥5% of an effect, effort, or both, is made up of or is attributable to a step (e.g., as in “some of the method is performed by step Y”) or indicates a step is performed ≥2 times (e.g., as in “step X is repeated some number of times”). Terms such as “predominately,” “most,” or “mostly,” (and “primarily” when not used to refer to an order of events or “mainly”) means detectably ≥50% (e.g., mostly comprises, predominately includes, etc., mean ≥50%) (e.g., a system that mostly includes element X is composed of >50% of element X). The term “generally” means ≥75% (e.g., generally consists of, generally associated with, generally comprises, etc., means ≥75%) (e.g., a method that generally consists of step X means that 75% of the effort or effect of the method is attributable to step X). “Substantially” or “nearly” uncontradicted means ≥95% (e.g., nearly all, substantially consists of, etc., mean ≥95%) (e.g., a collection that nearly entirely is made up of element X means that at least 95% of the elements in the collection are element X). Terms such as “generally free” of an element or “generally lacking” an element mean comprising ≤25˜% of an element and, uncontradicted, terms such as “substantially free” of an element mean comprising ≤˜5% of an element.

Uncontradicted, any aspect described with respect to an optionally present element(s)/step(s) also provides implicit support for corresponding aspect(s) in which one, some, most, generally all, nearly all, essentially all, or all of such element(s) are lacking/step(s) not performed, in respect of the relevant aspect. E.g., disclosure of a system comprising element X implicitly also supports a system lacking element X. That is, readers will understand that any element, feature, step, or characteristic of any aspect of the invention recited herein as being present in an aspect also implicitly provides support for the element, feature, step, or characteristic as being excluded from a corresponding/similar aspect of the invention implicitly disclosed by the explicit positive disclosure.

Uncontradicted, readers should interpret terms such as “essentially all” or “essentially” consistent with the concept of “consisting essentially of.”

Uncontradicted, changes to tense or presentation of terms (e.g., using “comprises predominately” in place of “predominately comprises”) do not change the meaning of the corresponding term/phrase.

Uncontradicted, all methods provided here can be performed in any suitable order regardless of presentation (e.g., a method comprising steps A, B, and C, can be performed in the order C, B, and A; B and A and C simultaneously, etc.). Uncontradicted, elements of a composition can be assembled in any suitable manner by any suitable method. In general, any methods and materials similar or equivalent to those described here can be used in the practice of embodiments. Uncontradicted, the use of ordinal numbers such as “first,” “second,” “third,” and so on to distinguish respective elements rather than to denote a particular order of elements.

Uncontradicted, any elements, steps, components, or features of aspects and all variations thereof, etc., are within the scope of the invention and the broadest version of any aspect.

Except where explicitly indicated or clearly indicated by context, terms such as “better” or “improved” herein mean detectably or significantly “increased,” when the increase is beneficial to a patient/user or with respect to desirable properties of a composition or method. In aspects, “improved” means DoS “reduced,” such as with respect to the toxicity of a composition. Uncontradicted, terms such as “enhanced,” “improved,” and the like are used synonymously.

Any elements associated with a function can be alternatively described as “means for” performing a function in a composition/device/system or a “step for” performing a part of a method, and parts of this disclosure refer to “equivalents,” which means known equivalents known in the art for achieving a referenced function associated with disclosed mean(s)/step(s). However, no element of this disclosure or claim should be interpreted as limited to a “means-plus-function” or “step-plus-function” construction unless such intent is clearly indicated by the use of the terms “means for” or “step for.” Terms such as “configured to” or “adapted to” do not indicate “means-plus-function” interpretation but, rather, describe element(s)/step(s) configured to, designed to, selected to, or adapted to achieve a certain performance, characteristic, property, or the like using teachings provided here or in the art.

As used herein, the word “exemplary” means “serving as an example, instance, or illustration.” The following detailed description is merely exemplary in nature and is not intended to limit application and uses. Any embodiment described herein as “exemplary” is not necessarily to be construed as preferred or advantageous over other embodiments.

All references (e.g., publications, patent applications, and patents) cited herein are hereby incorporated by reference as if each reference were individually and specifically indicated to be incorporated by reference and set forth in its entirety herein. Uncontradicted, any suitable principles, methods, or elements of such references (collectively “teachings”) can be combined with or adapted to aspects. However, citation/incorporation of patent documents is limited to the technical disclosure thereof and does not reflect any view regarding the validity, patentability, etc., thereof. In the event of any conflict between this disclosure and the teachings of such documents, the content of this disclosure controls regarding aspects of the invention. Numerous references are cited here to concisely incorporate known information and aid skilled persons in putting aspects into practice. While efforts have been made to include the most relevant references for such purposes, readers will understand that not every aspect of every cited reference will apply to every aspect of the invention. The invention is, in some respects, intended to provide device(s), system(s), method(s), or composition(s) that are distinguishable from (inventive with respect to) the prior art. Accordingly, while all references herein are incorporated by reference, readers will likewise understand that this disclosure provides for the explicit exclusion of any element disclosed in such prior art references that differs from the applicable elements of any aspect of the invention described herein.

While elements disclosed in such incorporated references can be combined with aspects of the invention disclosed herein, readers will understand that the invention is intended to stand apart from such disclosures and, accordingly, uncontradicted, in aspects, any element(s) of the objects or methods of any such references are excluded from the scope of the invention (e.g., if reference A discloses object or element B, any aspect that is not directed to object or element B can be characterized by, as one aspect, the lack of object or element B).

### Additional Terms, Concepts, and Acronyms

Uncontradicted, any description of the weight of compounds, ingredients, etc., of a composition described here is in reference to the weight percent (“wt. %”). As used herein, the term “w/w” means the weight ratio of one ingredient to another.

Compositions and practices described herein are typically pharmaceutically suitable. “Pharmaceutical suitability”, “pharmaceutically suitable,” and the like, are phrases typically used to refer to compositions/formulations or practices that are safe and effective for pharmaceutical administration and application, having sufficient potency, purity, strength, quality, and safety for pharmaceutical application, as may be judged by regulatory authority review, and as established by, e.g., one or more well-controlled and adequate clinical studies performed in compliance with generally prevailing regulatory authority standards. Typically, a “pharmaceutically acceptable” material refers to a material that is suitable for use in contact with the tissues of humans and lower animals without undue toxicity, irritation, allergic response, and the like, in keeping with a reasonable benefit-risk ratio, and effective for their intended use.

Uncontradicted, phrases such as “substantially identical” or “substantially similar” may be used to refer to element(s)/component(s)/ingredient(s)/thing(s) (e.g., composition, system, device, etc.) or step(s)/method(s) that have the same or about the same characteristic(s) or achieve the same or about the same result(s), typically in a similar way, as a referenced element/thing or step/method or otherwise do not meaningfully differ in intended result and manner of achieving such a result or are otherwise recognized in the art as not differing or not differing substantially in the relevant context (e.g., by being considered equivalents). Uncontradicted, readers will understand that a “substantially identical” or “substantially similar” element/thing or step/method when compared to a comparator thing/element or method/step means that the referenced element/thing or step/method exhibits such a similar function as a comparator at identical, approximately identical, or statistically similar amounts as the comparator thing or method when applied under similar conditions of use. Again, where statistical, approximate, or other measured comparisons are not possible, readers will understand the phrase as encompassing those things known as being identical or substantially identical to the referenced element/step or are described as such herein. In aspects, substantially similar or substantially identical elements exhibit no significant (i.e., statistically significant) difference from a referenced element.

Uncontradicted, terms such as “carrier” and “excipients” mean any physiologically inert, pharmacologically inactive material known to one skilled in the art, which is compatible with the physical and chemical characteristics of mexiletine compounds, generally, or the particular mexiletine compound or pharmaceutically acceptable salt described in connection therewith as an active ingredient. Pharmaceutically acceptable excipients include polymers, resins, plasticizers, fillers, binders, lubricants, glidants, disintegrants, suspending agents, deflocculating agents, solvents, co-solvents, buffer systems, surfactants, preservatives, sweetening agents, flavoring agents, pharmaceutical grade dyes or pigments, and viscosity agents, which are typically present in amounts suitable for providing a DoS effect consistent with such classification(s) of excipients.

Constituents of compositions/formulations described herein are typically present in “effective amounts,” and uncontradicted, any described class/type of, e.g., excipient (often referred to as a “component” herein—e.g., a “suspension component” which may include one or more suspension agent(s)/constituent(s), wherein each suspension agent may be referred to as a constituent of the suspension component, a constituent of the composition(s)/formulation(s) or both) or specific excipient, or, e.g., in certain aspects active pharmaceutical ingredient(s) (API(s) or an API component) is understood to be present in the associated composition/formulation in an effective amount, which generally means, in this context, an amount that is effective for the described function(s) associated with the excipient/API (it being understood that some excipient or API compound(s)/ingredient(s) exhibit more than one effect). E.g., a suspension agent will be understood to be present in a composition/formulation in an amount that is effective to impart an indicated suspension effect, a suspension effect that is required for suitability of the composition, or an effect that imparts a detectable or significant suspension-maintaining effect alone or in combination with other suspension component constituent(s) on a composition (with respect to a comparator composition lacking the compound(s)/ingredient(s)).

Steps, elements, devices, components, and the like also or alternatively can be described as “effective.” Uncontradicted, any step performed, function of a component, etc., is to be understood as being applied effectively, such as in an effective amount, which generally means, in this context, an amount that is effective for the described function(s) associated with the component, device, step, etc. Efficacy in this respect can mean, and, uncontradicted, should be interpreted to implicitly disclose efficacy in terms of (1) in a treated subject, (2) in a majority of subjects in a population, (3) in a statistically significant number of subjects in a population, (4) generally all subjects in a population, (5) substantially all subjects in a population, or (6) in a statistically significant number of or more of a typical or average subject of the class of subjects to be treated in the condition (treatment in this sense encompassing any administration, application, etc., whether for therapeutic reasons or prophylactic/maintenance reasons).

As used herein, terms such as “pharmaceutical composition,” “formulation,” and the like, when used to describe compositions generally described or referenced herein, include solid dosage forms such as granules, multiunit particulate systems (MUPS), pellets, spheres, tablets, dispersible tablets, soft capsules, hard capsules, mini-tablets, beads, particles and the like; and liquid dosage forms such as solutions, suspensions, emulsions, colloids and the like, meant for enteral administration; and, further, can encompass dosage forms suitable for transdermal or transmucosal administration.

Excipients and active pharmaceutical ingredients (e.g., mexiletine) described in aspects herein are typically “effective” and present in “effective amounts,” and, uncontradicted, any described class of excipient or specific excipient is understood to be present in the associated composition/formulation in an effective amount, which generally means, in this context, an amount that is effective for the described function(s) associated with the excipient (it being understood that some excipient compound(s)/ingredient(s) exhibit more than one effect).

Uncontradicted, the term “standard” as used herein with respect to techniques or components/elements not further described or exemplified means either the standard applied by leading regulatory authorities (e.g., US FDA, the EU EMA, the Chinese NMPA, Japan's PMDA, Health Canada, India's CDSCO, and the like) or that is most commonly used in the art.

Uncontradicted, any reference to a compound herein that can form salts is to be interpreted as simultaneously implicitly disclosing the compound and any or all suitable pharmaceutically acceptable salts thereof. The term “pharmaceutically acceptable salt” includes suitable salts prepared from pharmaceutically acceptable bases or acids including inorganic or organic bases and inorganic or organic acids. Examples of such salts that can be suitable in contexts include acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate, diphosphate, polygalacturonate, salicylate, stearate, sulfate, subacetate, succinate, tannate, tartrate, teoclate, tosylate, triethiodide, and valerate. Examples of salts derived from inorganic bases include but are not limited to, aluminum, ammonium, calcium, copper, ferric, ferrous, lithium, magnesium, manganic, mangamous, potassium, sodium, and zinc.

Uncontradicted, the term “controlled release” means the time course of drug appearance in medium surrounding the composition is DOS modified, typically significantly modified, as compared to an otherwise corresponding/related immediate release (IR) composition. Uncontradicted, immediate-release compositions exhibit detectable or significant release within minutes of administration and typically achieve ≥85% of release (e.g., ˜80%, ˜90%, or ˜95%) within ˜30 minutes of administration (e.g., within ˜20, ˜25, ˜35, or ˜40 minutes of administration). Controlled release encompasses “delayed release” and “extended-release” formulations. Uncontradicted, the term “extended release” refers to compositions that are characterized by having a detectable or significant release over a period of at least ˜24 hours. Delayed-release formulations can include a release of part of a delivered active pharmaceutical ingredient (API) beyond immediate release.

As used herein, the term “T1 or Test product 1” refers to a product having composition 1 as described in a suitable example. As used herein, the term “T2 or Test product 2” refers to a product having composition 2 as described in a suitable example. As used herein, the term “T3 or Test product 3” refers to a product having composition 3 as described in a suitable example.

Uncontradicted, a “subject” means a mammal, such as, for example, a human, e.g., a human patient having a condition treatable by receiving an effective amount of a composition of the invention an effective number of times. In aspects, subjects are limited to those mammals that would exhibit responses to a composition that is indicative of efficacy of the same composition in humans.

The “relative bioavailability” can be understood as the ratio of the plasma or serum AUC determined from a plot of the plasma or serum drug concentration versus time measured for the composition or method of the present composition to the plasma or serum AUC of a control composition. The term “AUC₀₋₂₄” can be understood as the area under the curve relating blood plasma concentration to time after administration from 0 to 24 hours, as determined using the linear trapezoidal rule, and are expressed in units of (ng*h/ml). The term “AUC_{0-∞}” can be understood as the area under the curve relating blood plasma concentration to time from time 0 hours to infinity and is expressed in units of (ng*h/ml). The term “AUC_{0-τ}” can be understood as the area under the blood plasma concentration to time curve from time zero to time tau over a dosing interval at steady state, where tau is the length of the dosing interval and is expressed in units of (ng*h/ml). The term “Cₘₐₓ” can be understood as the maximum observed blood plasma concentration or the maximum blood plasma concentration calculated or estimated from a concentration to time curve, and is expressed in units of ng/ml. The term “Tₘₐₓ” can be understood as the time after administration at which Cₘₐₓ occurs and is expressed in units of hours (h). Terms like “single dose” can be understood to indicate a human patient has received a single dose of the drug formulation and the drug plasma concentration has not achieved a steady state (e.g., 7.5 mL of 80 mg/mL of a suspension as described herein). The “steady state” can be understood as the blood plasma concentration curve for a given drug does not substantially fluctuate after repeated doses of the formulation. The elimination rate constant “Kel” can be understood to describe the fraction of drug eliminated per unit of time or the rate at which plasma concentrations will decline during the elimination phase. The term “half-life (t_{1/2})” can be understood as referring to the time required for plasma concentration of a drug to decrease by 50% t_{1/2} is dependent on the rate constant (k). The term “food effect” can be understood as meaning the relative difference in AUC, Cₘₐₓ, and/or Tₘₐₓ of an active substance, when said substance or a formulation thereof, such as a tablet, a capsule, or a suspension, is administered orally to a mammal, preferably a human, concomitantly with food or in a fed state as compared to the same values when the same formulation is administered in a fasted state. The food effect F can be calculated as, e.g., F=(Y_{fed}−Y_{fasted})/Y_{fasted}; wherein Y_{fed} and Y_{fasted} are the found values of AUC, Cₘₐₓ, or Tₘₐₓ in the fed and fasted state, respectively.

The term “myotonia” can generally be understood to include dystrophic and/or non-dystrophic myotonic disorders as well as myotonic dystrophy type 1 and type 2. Examples of such myotonic disorders are provided in, e.g., EMA 2018. However, each such condition also can be a particular aspect, at the exclusion of one, some, or all such other conditions. The invention also includes methods for treating symptomatic treatment of myotonia in adult patients with non-dystrophic and/or dystrophic myotonic disorders.

Uncontradicted, an “ion exchange resin” can be understood to mean either or both anionic or cationic ion exchange resins. Uncontradicted, a “drug ion-exchange resin complex” can be understood as a drug-containing ion-exchange resin particle in which there is an ionic bond between the drug and the ion-exchange resin particle. Uncontradicted, a “mexiletine ion exchange resin complex” means a complex formed by loading mexiletine or its pharmaceutically acceptable salt onto an ion exchange resin.

### SUMMARY OF THE INVENTION

This “Summary of the Invention” section (“Summary”) briefly describes the elements and characteristics of selected illustrative embodiment(s) of the invention. The brief summaries of such embodiments provided here are primarily intended to illustrate the nature of the invention and, accordingly, the content of this Summary is not intended to be all-inclusive, and the scope of the invention is not limited to, or by, the exemplary aspects of the invention provided in this section. Any of the aspects of the invention described in this section can be combined with any other aspect described in this or any other aspect of this disclosure.

In one aspect, the invention provides pharmaceutical compositions that are adapted for oral administration and that comprise a therapeutically effective amount of mexiletine or a pharmaceutically acceptable salt thereof and a release retarding agent, wherein the release retarding agent effectively controls the release of the mexiletine or pharmaceutically salt thereof from the composition such that the release retarding agent causes the mexiletine or a pharmaceutically acceptable salt thereof to be released from the composition after administration to human patients at a rate such that a single administration of the composition administered daily is effective to treat myotonia in a statistically significant number of myotonia patients over a treatment period of at least about a month, such as at least about three months (e.g., at least about 6, 12, 18, 24, 30, or 36 months, or longer). In aspects, the invention provides methods of using such compositions to treat conditions, such as myotonia, comprising administering an effective amount of such a composition to a patient, once a day, for a period, such as a period of at least 1 month, or 3, 6, 9, 12, 18, 24, 30, or 36 months, or longer, wherein the method does not comprise administering the composition to the patient more than once a day. In aspects, the methods and compositions are at least about as effective, if not detectably or significantly more effective, than comparator products, such as the on-market product known as Namuscla which is described further herein and in the EMA 2018 document cited in the Background (EMA 2018). Such compositions are distinguishable from Namuscla in being effective throughout a treatment period with only once-daily administration. In aspects, the number of patients in which once-daily administration of the composition of the invention is effective is significantly greater than the number of patients treatable with once-daily administration of Namuscla.

In aspects, each dose of a composition as described in the preceding paragraph comprises about 100-about 600 mg of mexiletine hydrochloride, such as about 200-about 600 mg of mexiletine hydrochloride (e.g., ˜225-600 mg, ˜250-600 mg, ˜275-600 mg, ˜300-600 mg, ˜350-600 mg, ˜400-600 mg, or ˜450-600 mg, such as ˜100-550 mg, ˜100-500 mg, ˜100-450 mg, ˜100-400 mg, ˜100-300 mg, or ˜100-250 mg. In aspects, each dose of a composition comprises at least ˜200, 250, or ˜300 mg to no more than ˜600 mg, 550 mg, 500 mg, 450 mg, 400 mg, or ˜350 mg.

In aspects, the invention provides compositions according to either of the preceding paragraphs of this section, wherein the composition can be further characterized in exhibiting a median, Tₘ of about 5 to about 8 hours (e.g., about 5.5-7.5 hours, such as about 6-7, about 5.75-6.75, about 5.75-6.25, or about 6 hours); a mean t_{1/2} about 10 hours to about 13.5 hours (e.g., about 10.5-13 hours, about 11-12.5 hours, about 10.5-12.5 hours, about 11-12 hours, about 10.5-12 hours, about 11 hours, about 12 hours, about 11-13 hours, or about 11-13.5 hours); or both such characteristics.

The invention further provides compositions according to any of the preceding passages of this section, wherein the composition is further characterized in exhibiting at least one of the following pharmacokinetic profile characteristics after single dose administration: (a) a mean Cₘₐₓ of about 700-1250 ng/mL; a mean AUC0-24 about 12000 ng*hr/mL to about 17500 ng*hr/mL; a mean AUC₀₋ₜ of about 22000-24250 ng*hr/mL; and a mean AUC_{0-∞} about 22500-26000 ng*hr/mL.

In further aspects, the invention provides pharmaceutical compositions comprising an effective amount of mexiletine or a pharmaceutically acceptable salt thereof and an effective amount of a release retardant/retarding agent wherein the composition also (in combination with any other aspects of this section or disclosure) or alternatively (to any such aspects) exhibits at least one, such as at least two, such as 3 or more (e.g., 1-5, 2-5, 2-4, 2-3, 1-4, 1-3, 3, 4, 5, or all) of the following pharmacokinetic profile characteristics after single dose administration:
(a) median, Tₘₐₓ about 5 to about 8 hours;
(b) mean, Cₘₐₓ about 700-1250 ng/mL;
(c) mean, AUC0-24 about 12000 ng*hr/mL to about 17500 ng*hr/mL;
(d) mean, AUC₀₋ₜ of about 22000-24250 ng*hr/mL;
(e) mean, AUC_{0-∞} about 22500-26000 ng*hr/mL; and
(f) mean, t_{1/2} about 10 hours to about 13.5 hours.

In aspects, the compositions of the invention, such as the compositions described in any of the preceding parts of this section, further exhibit an increase in mean Cmax, mean AUC, or both, in a fed condition with respect to fasting condition.

In aspects, the composition of the invention exhibits one, two, three, or more of any such pharmacokinetic profile characteristics, wherein the applicable pharmacokinetic profile characteristic is significantly or detectably different from the corresponding characteristic obtained by TID administration of 167 mg of Namuscla.

In aspects, the compositions of the invention, such as compositions described in any of the preceding passages of this section, exhibit a dissolution profile in a suitable dissolution media, such that those exemplified herein or otherwise known in the art, in which about 10-40% of the mexiletine in the composition is released within 4 hours, about 30-70% is released within about 12 hours, and more than about 60%, 70%, or more than about 80% of the mexiletine in the composition is released within 24 hours.

In aspects, the invention provides orally-administrable mexiletine controlled-release once-a-day liquid suspension of mexiletine or a pharmaceutical acceptable salt thereof that enables an early onset of therapeutic effect or efficacy for an extended period of about 24 hours while maintaining drug bioavailability (e.g., at least about 70% bioavailability, at least about 80% bioavailability, such as ˜60-95%, ˜65-90%, 65-85%, 70-90%, ˜75-90%, 70-85%, or 75-85% bioavailability, etc.), e.g., for the treatment of myotonia.

In some embodiment, the present invention discloses an oral controlled release once a day pharmaceutical composition comprising mexiletine or pharmaceutically acceptable salts thereof wherein, the pharmaceutical compositions exhibit at least about 80% relative bioavailability based on the area under the plasma concentration curve (AUC) in human subjects under fasting and fed condition, while compared to commercially available Namuscla® 167 mg immediate-release capsules administered thrice a day.

In some embodiment, the present invention discloses an oral controlled release once daily pharmaceutical composition comprising mexiletine or pharmaceutically acceptable salts thereof, wherein such pharmaceutical composition exhibits an increase in Cmax and AUC in a standard fed condition compared to a standard fasted condition.

In some embodiment, the present invention discloses an oral controlled release once daily pharmaceutical composition according to any one or more of the preceding aspects described in this section comprising about 150 mg to about 600 mg of mexiletine or pharmaceutically acceptable salts thereof (e.g., about 150-550, 150-500, 150-450, 150-400, 150-350, 200-550, 200-500, 200-450, 200-400, 250-600, 250-550, 250-500, 250-450, 250-400, 300-500, 300-450, or 300-400 mg of mexiletine or a pharmaceutically acceptable salt thereof).

This disclosure provides a number of different means for achieving formulations/compositions having such properties. One exemplary aspect relates to an oral controlled release once daily pharmaceutical compositions comprising mexiletine or a pharmaceutically acceptable salt thereof, a pharmaceutically acceptable ion exchange resin, and a pharmaceutically acceptable carrier, which exhibits one or more characteristics described in the preceding paragraphs of this section.

Another aspect of the invention relates to an oral controlled release once daily pharmaceutical composition comprising mexiletine or a pharmaceutically acceptable salt thereof, a pharmaceutically acceptable ion exchange resin, and a pharmaceutically acceptable carrier wherein, the said composition further comprises at least one release retarding agent.

Another aspect of the invention relates to an oral controlled release once daily pharmaceutical composition containing an ion exchange resin having mexiletine or pharmaceutical acceptable salt thereof adsorbed thereon to provide a mexiletine ion exchange resin complex wherein the complex is further coated with at least one release retarding agent to enhance the control of mexiletine release from the mexiletine ion exchange resin complex.

Another aspect of the invention relates to a process for preparing an oral controlled release once daily pharmaceutical composition of mexiletine or pharmaceutical acceptable salt thereof wherein, said process comprises; (i) preparing a complex comprising mexiletine or a pharmaceutical acceptable salt thereof and a pharmaceutically acceptable ion exchange resins and one or more pharmaceutically acceptable excipients; (ii) dissolving and/or dispersing a release retarding agent and one or more pharmaceutically acceptable coating additives in a suitable solvent to prepare a coating composition; and (iii) applying the coating composition onto the complex of mexiletine and ion exchange resin to obtain a controlled release once daily composition of mexiletine or a pharmaceutical salt thereof.

Another aspect of the invention relates to an oral controlled release once daily pharmaceutical composition comprising mexiletine or a pharmaceutically acceptable salt thereof, in the form of powder or granules which may be mixed with suspension base to form an orally administrable controlled release aqueous suspension.

Another aspect of the invention relates to a process for preparing an oral controlled release once daily liquid suspension composition of mexiletine or pharmaceutical acceptable salt thereof wherein, said process comprises (i) loading of mexiletine or a pharmaceutical salt thereof onto a pharmaceutically acceptable ion exchange resin to obtain mexiletine ion exchange resin complex; (ii) coating the mexiletine ion exchange resin complex with a suitable release retarding agent; preferably a hydrophilic and/or hydrophobic release retardant polymer and (iii) suspending the coated complex into a suspension base to obtain controlled release liquid suspension of mexiletine.

Another aspect of the present invention relates to a process of preparing a controlled release once a daily dry powder for liquid suspension of mexiletine or a pharmaceutically acceptable salt thereof involves steps including (i) loading of mexiletine or pharmaceutical acceptable salt thereof onto a pharmaceutically acceptable ion exchange resin to obtain mexiletine ion exchange resin complex; (ii) coating the mexiletine ion exchange resin complex with a suitable release retarding agent; preferably a hydrophilic and/or hydrophobic release retardant polymer and (iii) suspending the coated complex into a suspension base to obtain dry powder of controlled release composition to be re-suspended using suitable amount of water to obtain controlled release liquid suspension of mexiletine.

Another aspect of the present invention relates to a process of preparing a controlled release once a daily dry powder for liquid suspension of mexiletine or a pharmaceutical acceptable salt thereof involves steps including (i) loading of mexiletine or a pharmaceutically acceptable salt thereof onto a pharmaceutically acceptable ion exchange resin to obtain mexiletine ion exchange resin complex; (ii) coating the mexiletine ion exchange resin complex with a suitable release retarding agent; preferably a combination of hydroxypropyl methylcellulose and ethyl cellulose and (iii) suspending the coated complex into a suspension base to obtain dry powder of controlled release composition to be re-suspended using suitable amount of water to obtain controlled release liquid suspension of mexiletine.

Another aspect of the present invention relates to a process of preparing a controlled release once a daily dry powder for liquid suspension of mexiletine or a pharmaceutical acceptable salt thereof involves steps including (i) loading of mexiletine or a pharmaceutically acceptable salt thereof onto a pharmaceutically acceptable ion exchange resin to form a resin complex; (ii) coating the mexiletine ion exchange resin complex with a suitable release retarding agent; preferably a hydrophilic and/or hydrophobic release retardant polymer and (iii) suspending the coated complex into a suspension base, wherein the suspension base comprises one or more suspending agents, one or more glidants, one or more viscosity increasing agents, one or more diluents, one or more sweetening agents, one or more flavoring agents, and one or more preservatives.

Another aspect of the invention relates to an oral controlled release once a daily pharmaceutical composition comprising mexiletine or a pharmaceutically acceptable salt thereof, a pharmaceutically acceptable ion exchange resin, and one or more pharmaceutically acceptable excipients wherein, pharmaceutically acceptable excipients are selected from the group consisting of one or more hydrophilic and/or hydrophobic release retardant polymer, one or more plasticizer, one or more vehicles, one or more suspending agents, one or more glidants, one or more viscosity increasing agents, one or more diluents, one or more sweetening agents, one or more flavoring agents, and one or more preservatives.

Another aspect of the invention relates to an oral controlled release once a daily pharmaceutical composition comprising mexiletine or a pharmaceutically acceptable salt thereof, a pharmaceutically acceptable ion exchange resin, and a pharmaceutically acceptable carrier wherein the ratio of mexiletine to ion exchange resin ranges from about 1:0.25 to about 1:10.

Another aspect of the invention relates to an oral controlled release once daily liquid pharmaceutical composition comprising mexiletine or a pharmaceutically acceptable salt, a pharmaceutically acceptable ion exchange resin and a pharmaceutically acceptable release retarding polymer wherein at least about 30% to about 85% of mexiletine is released in vitro from about 8 hours to about 24 hours in USP type IV apparatus having flow rate 8 ml/min along with 6 gm glass beads in 0.1N HCl media, pH 5.0 acetate buffer media and in pH 6.8 phosphate buffer media at 37° C.±0.5° temp respectively.

Additional embodiments of the present compositions and methods and the like will be apparent from the following description, and examples. As can be appreciated from the foregoing and following description, each feature described herein, and each combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description, particularly when considered in conjunction with the accompanying examples.

### BRIEF DESCRIPTION OF THE DRAWINGS/FIGURES

The drawings/figures provided here, and any associated brief description of figures provided below, are intended to exemplify certain aspects and principles of the invention without limiting its scope.

FIG. 1 is a linear mean plot of plasma concentration (ng/mL) of mexiletine vs time after administration (hrs.) for a Reference Product and two test products (Test Product 1 and Test Product 2) (each being exemplary compositions of the invention), as described in the Examples.

FIG. 2 is a linear mean plot of plasma concentration (ng/mL) of mexiletine vs. time (hrs.) measured in a food effect study using an exemplary composition of the invention as described in the Examples.

### EXEMPLARY ASPECTS OF THE INVENTION

The following is a non-limiting list of exemplary aspects of the invention, which illustrates embodiments of the invention in a summary form to aid readers in quickly understanding the overall scope of the invention. Similar to patent claims, listed aspects described in the paragraphs of this section may make reference to (depend on/from) one or more other paragraphs. Readers will understand that such references mean that the features/characteristics or steps of such referenced aspects are incorporated into/combined with the referring aspect. E.g., if an aspect in a paragraph (e.g., a paragraph indicated by text at the end of the paragraph as aspect 2) refers to another aspect by one or more aspect numbers (e.g., aspect 1 or “any one of aspects 1-3”), it will be understood to include the elements, steps, or characteristics of such referenced aspects (e.g., aspect 1) in addition to those of the aspect in which the reference is made (e.g., if aspect 2 refers to aspect 1, it provides a description of a composition, method, system, device, etc., including the features of both aspect 1 and aspect 2).

Lists of aspects describing specific exemplary embodiments of the invention are sometimes employed to aid the reader in understanding the invention. Such aspects can, within them, reference other exemplary aspects, either individually or as groups of aspects (e.g., via reference to a range within a list of numbered aspects when such aspects are provided as a numbered list). Reference to ranges of aspects should be interpreted as referencing all such aspects individually, each as unique embodiments of the invention, and in combination with one another as unique embodiment(s) of the invention, according to the presentation provided of such aspects unless such an aspect within such a referenced range is either contradictory or non-sensical. If contradicted, reference to the contradictory aspect should be excluded.

A first set of exemplary aspects is provided here:

In aspects, the invention provides a pharmaceutical composition formulated for oral administration comprising a therapeutically effective amount of mexiletine or pharmaceutically acceptable salt thereof and an effective amount of a release retarding agent, wherein the release retarding agent causes the mexiletine or pharmaceutically acceptable salt thereof to be released from the composition after administration to human patients at a rate such that a single administration of the composition administered daily is effective to treat myotonia in a statistically significant number of myotonia patients over a treatment period of at least about three months (aspect A).

In aspects, the invention provides the composition of aspect A, wherein the composition comprises about 100-about 600 mg of mexiletine hydrochloride (aspect B).

In aspects, the invention provides the composition of any one or both of aspect A or aspect B, wherein the composition comprises about 200-about 600 mg of mexiletine hydrochloride (aspect C).

In aspects, the invention provides the composition of any one or more of aspects A-C, wherein the composition exhibits a median, Tₘₐₓ of about 5 to about 8 hours, the composition exhibits a mean t_{1/2} of about 10 hours to about 13.5 hours, or both (aspect D).

In aspects, the invention provides the composition of any one or more of aspects A-D, wherein the composition further exhibits at least one of the following pharmacokinetic profile characteristics after single dose administration: a mean Cₘₐₓ of about 700-1250 ng/mL; a mean AUC0-24 of about 12000 ng*hr/mL to about 17500 ng*hr/mL; a mean AUC₀₋ₜ of about 22000-24250 ng*hr/mL; and a mean AUC_{0-∞} of about 22500-26000 ng*hr/mL (aspect E).

In aspects, the invention provides the composition of any one or more of aspects A-E, wherein the composition exhibits at least two of the following pharmacokinetic profile characteristics after single dose administration: a median Tₘₐₓ of about 5 to about 8 hours; a mean Cₘₐₓ of about 700-1250 ng/mL; a mean AUC0-24 of about 12000 ng*hr/mL to about 17500 ng*hr/mL; a mean AUC₀₋ₜ of about 22000-24250 ng*hr/mL; a mean AUC_{0-∞} of about 22500-26000 ng*hr/mL; and a mean, t_{1/2} of about 10 hours to about 13.5 hours (aspect F).

In aspects, the invention provides the composition of any one or more of aspects A-F, wherein the pharmaceutical composition exhibits an increase in mean Cmax, mean AUC, or both, in a fed condition compared to a fasting condition (aspect G).

In aspects, the invention provides the composition of any one or more of aspects A-G, wherein the composition exhibits an in vitro dissolution profile characterized by
(a) (1) after 0-2 hours, from about 0% to about 30% by weight of mexiletine is released from the composition, after 0-4 hours from about 10% to about 40% by weight of mexiletine is released from the composition, (2) after 4-12 hours from about 30% to about 70% by weight of mexiletine is released from the composition, (3) from 6-14 hours more than about 60% by weight of mexiletine is released from the composition, and (4) more than about 80% by weight of mexiletine is released from the composition within about 24 hours, when measured using a USP type IV apparatus having flow rate 8 ml/min along with 6 gm glass beads in 0.1N HCl media, 37° C.±0.5° C.;
(b) (1) after 0-2 hours from about 0% to about 25% by weight of mexiletine is released from the composition, (2) after 0-4 hours from about 10% to about 40% by weight of mexiletine is released from the composition, and (3) after 4-12 hours from about 30% to about 100% of the mexiletine is released from the composition within about 24 hours when measured using a USP type IV apparatus having flow rate 8 ml/min along with 6 gm glass beads in pH 5.0 acetate buffer media, 37° C.±0.5° C.;
(c) (1) after 0-2 hours from about 0% to about 35% by weight of mexiletine is released from the composition, (2) after 0-4 hours from about 10% to about 50% by weight of mexiletine is released from the composition, (3) after 4-12 hours from about 30% to about 100% from the composition, when measured using a USP type IV apparatus having flow rate 8 ml/min along with 6 gm glass beads in pH 6.8 phosphate buffer media, 37° C.±0.5° C.;
(d) the composition (1) releases not more than 50% by weight of mexiletine in an initial 1 hour in 500 mL 0.1N HCl and (2) followed by 700 mL pH 4.5 acetate buffer changeover media the said composition releases not less than 65% by weight of mexiletine in 2 hours and (3) further followed by 900 mL pH 6.8 phosphate buffer changeover media the composition releases not less than 85% by weight of mexiletine in 4 hours when measured in a United States Pharmacopoeia (USP) type II dissolution apparatus, rotated at 75 rpm at a temperature of 370±0.5° C.; or
(e) a combination of any or all of (a)-(d) (aspect H).

In aspects, the invention provides the composition of any one or more of aspects A-H, wherein upon a single oral administration of the composition (1) Tmax occurs between about 5 to about 8 hours after administration in most human patients and (2) the plasma concentration of mexiletine in most human patients 16 hours after administration of the composition is within at least about 33% of Cmax (aspect I).

In aspects, the invention provides the composition of any one or more of aspects A-I, wherein the plasma concentration in most human patients receiving a single oral administration of the composition remains within about 20% of Cmax for a period of at least about 4 hours (aspect J).

In aspects, the invention provides the composition of any one or more of aspects A-J, wherein the plasma concentration of mexiletine in most human patients receiving a single oral administration of the compound at either 3 hours after administration, 4 hours after administration, or both, is within about 25% of the plasma concentration of mexiletine 18 hours after administration, 20 hours after administration, or both (aspect K).

In aspects, the invention provides a pharmaceutical composition formulated for oral administration comprising a therapeutically effective amount of mexiletine or pharmaceutically acceptable salt thereof and an effective amount of a release retarding agent, wherein the composition exhibits at least one of the following pharmacokinetic profile characteristics after single dose administration: a median Tₘₐₓ of about 5 to about 8 hours; a mean Cₘₐₓ of about 700-1250 ng/mL; a mean AUC0-24 of about 12000 ng*hr/mL to about 17500 ng*hr/mL; a mean AUC₀₋ₜ of about 22000-24250 ng*hr/mL; a mean AUC_{0-∞} of about 22500-26000 ng*hr/mL; and (f) a mean t½ about 10 hours to about 13.5 hours (aspect L).

In aspects, the invention provides the composition of aspect L, wherein the composition exhibits a mean t_{1/2} of about 10 hours to about 13.5 hours, a median, Tₘₐₓ of about 5 to about 8 hours, or both (aspect M).

In aspects, the invention provides the composition of any one or both of aspect L or aspect M, wherein the pharmaceutical composition exhibits an increase in mean Cmax, mean AUC, or both, in a fed condition with respect to fasting condition (aspect N).

In aspects, the invention provides the use of a composition according to any one of aspects A-N for the preparation of a medicament to treat myotonia (aspect O).

In aspects, the invention provides a method of treating myotonia in a patient in need thereof comprising administering an effective amount of a composition according to any one of aspects A-O to a myotonia patient with food, once a day, for a treatment period of at least three months, wherein the method treats myotonia in a statistically significant number of myotonia patients (aspect P).

In aspects, the invention provides a pharmaceutical composition for oral administration comprising (a) a therapeutically effective amount of mexiletine or pharmaceutically acceptable salt thereof and (b) a pharmaceutically acceptable ion exchange resin and at least one or more pharmaceutically acceptable excipient (aspect Q).

In aspects, the invention provides the composition of aspect Q, wherein the ratio of mexiletine or pharmaceutically acceptable salt thereof to the pharmaceutically acceptable ion exchange resin in the composition is about 1:0.5 to about 1:5 (aspect R).

In aspects, the invention provides the composition of any one or both of aspect Q or aspect R, wherein the mexiletine or pharmaceutically acceptable salt thereof and the ion exchange resin form a pharmaceutically acceptable mexiletine resin complex wherein, the ion exchange resin is about 30% to about 85% of the mexiletine-resin complex on a weight-to-weight basis (aspect S).

In aspects, the invention provides the pharmaceutical composition of any one or more of aspects Q-S, wherein the composition further comprises (c) a suitable release retarding agent selected from a hydrophilic release retardant polymer, a hydrophobic release retardant polymer, or both (aspect T).

In aspects, the invention provides the composition of any one or more of aspects Q-T, wherein the composition further comprises a suspension base selected from starch or disaccharide or both (aspect U).

In aspects, the invention provides the composition of any one or more of aspects Q-U, wherein the composition comprises about 100-about 600 mg of mexiletine hydrochloride (aspect V).

In aspects, the invention provides the composition of any one or more of aspects Q-V, wherein the composition exhibits an in vitro dissolution profile characterized by
(a) (1) after 0-2 hours, from about 0% to about 30% by weight of mexiletine is released from the composition, after 0-4 hours from about 10% to about 40% by weight of mexiletine is released from the composition, (2) after 4-12 hours from about 30% to about 70% by weight of mexiletine is released from the composition, (3) from 6-14 hours more than about 60% by weight of mexiletine is released from the composition, and (4) more than about 80% by weight of mexiletine is released from the composition within about 24 hours, when measured using a USP type IV apparatus having flow rate 8 ml/min along with 6 gm glass beads in 0.1N HCl media, 37° C.±0.5° C.;
(b) (1) after 0-2 hours from about 0% to about 25% by weight of mexiletine is released from the composition, (2) after 0-4 hours from about 10% to about 40% by weight of mexiletine is released from the composition, and (3) after 4-12 hours from about 30% to about 100% of the mexiletine is released from the composition within about 24 hours when measured using a USP type IV apparatus having flow rate 8 ml/min along with 6 gm glass beads in pH 5.0 acetate buffer media, 37° C.±0.5° C.;
(c) (1) after 0-2 hours from about 0% to about 35% by weight of mexiletine is released from the composition, (2) after 0-4 hours from about 10% to about 50% by weight of mexiletine is released from the composition, (3) after 4-12 hours from about 30% to about 100% from the composition, when measured using a USP type IV apparatus having flow rate 8 ml/min along with 6 gm glass beads in pH 6.8 phosphate buffer media, 37° C.±0.5° C.;
(d) the composition (1) releases not more than 50% by weight of mexiletine in an initial 1 hour in 500 mL 0.1N HCl and (2) followed by 700 mL pH 4.5 acetate buffer changeover media, the composition releases not less than 65% by weight of mexiletine in 2 hours, and (3) further followed by 900 mL pH 6.8 phosphate buffer changeover media the composition releases not less than 85% by weight of mexiletine in 4 hours when measured in a United States Pharmacopoeia (USP) type II dissolution apparatus, rotated at 75 rpm at a temperature of 370±0.5° C.; or
(e) a combination of any or all of (a)-(d) (aspect W).

In aspects, the invention provides the composition of any one or more of aspects Q-W, wherein upon a single oral administration of the composition (1) Tmax occurs between about 5 to about 8 hours after administration in most human patients and (2) the plasma concentration of mexiletine in most human patients after 18 hours administration of the composition is at least about 33% Cmax (aspect X).

In aspects, the invention provides the composition of any one or more of aspects Q-X, wherein the plasma concentration in most human patients receiving a single oral administration of the composition remains within about 20% of Cmax for a period of at least about 4 hours (aspect Y).

In aspects, the invention provides the composition of any one or more of aspects Q-Y, wherein the plasma concentration of mexiletine in most human patients receiving a single oral administration of the compound at either 3 hours after administration, 4 hours after administration, or both, is within about 25% of the plasma concentration of mexiletine 18 hours after administration, 20 hours after administration, or both (aspect Z).

In aspects, the invention provides the composition of any one or more of aspects Q-Z, wherein the composition exhibits at least one of the following pharmacokinetic profile characteristics after single dose administration: a median Tₘₐₓ about 5 to about 8 hours; a mean Cₘₐₓ about 700-1250 ng/mL; a mean AUC0-24 about 12000 ng*hr/mL to about 17500 ng*hr/mL; a mean AUC₀₋ₜ of about 22000-24250 ng*hr/mL; a mean AUC_{0-∞} about 22500-26000 ng*hr/mL; and a mean t_{1/2} about 10 hours to about 13.5 hours (aspect AA).

In aspects, the invention provides the composition of any one or more of aspects Q-AA, wherein the pharmaceutical composition exhibits an increase in mean Cmax, mean AUC, or both, in a fed condition with respect to fasting condition (aspect AB).

In aspects, the invention provides the use of a composition according to any one of aspects Q-AB for the preparation of a medicament to treat myotonia (aspect AC).

In aspects, the invention provides a method of treating myotonia in a patient in need thereof comprising administering an effective amount of a composition according to any one of aspects Q-AC to a patient with food, once or twice a day, for a number of times suitable to treat one or more conditions or symptoms of myotonia in the patient (aspect AD).

In aspects, the invention provides a method of treating myotonia in a patient in need thereof comprising administering an effective (i.e., therapeutically effective) amount of a composition according to any one of aspect A-aspect O and aspect Q-aspect AC.

A second set of exemplary aspects is provided here:

In aspects, the invention provides a pharmaceutical composition comprising (a) a therapeutically effective amount of a pharmaceutically acceptable salt of mexiletine and (b) a pharmaceutically acceptable ion exchange resin, wherein the pharmaceutically acceptable salt of mexiletine and the ion exchange resin form a pharmaceutically acceptable drug-resin complex (aspect 1).

In aspects, the invention provides a pharmaceutical composition of aspect 1, wherein the composition further comprises (c) an effective amount of a suitable release retarding agent comprising an effective amount of a hydrophilic release retardant polymer, a hydrophobic release retardant polymer, or both, wherein the release retarding agent coats at least some of the drug-resin complex to form coated complex particles and detectably delays release of mexiletine from the coated complex particles (aspect 2).

In aspects, the invention provides a composition of any one or both of aspects 1-2, wherein the composition further comprises a suspension base that suspends coated complex particles to form a controlled release liquid suspension of mexiletine (aspect 3).

In aspects, the invention provides a composition of any one or more of aspects 1-3, wherein the ratio of the pharmaceutically acceptable salt of mexiletine to the pharmaceutically acceptable cationic exchange resin in the composition is about 1:0.5 to about 1:5 (aspect 4).

In aspects, the invention provides a composition of any one or more of aspects 1-4, wherein the cation exchange resin is at least mostly composed of a material having a pKa of about −1.5 or less (aspect 5).

In aspects, the invention provides a composition of any one or more of aspects 1-5, wherein at least generally all of the cation exchange resin comprises a sulfonate group (aspect 6).

In aspects, the invention provides a composition of any one or more of aspects 1-6, wherein at least generally all of the cation exchange resin comprises a polystyrene component (aspect 7).

In aspects, the invention provides a composition of any one or more of aspects 1-7, wherein the cation exchange resin makes up about 30% to about 85% of the drug-resin complex on a weight-to-weight basis (aspect 8).

In aspects, the invention provides a composition of any one or more of aspects 1-8, wherein at least generally all the cation exchange resin is composed of (or substantially composed of or only composed of) sodium polystyrene sulfonate (aspect 9).

In aspects, the invention provides a composition of any one or more of aspects 1-9, wherein the mexiletine salt makes up about 15% to about 70% of the drug-resin complex on a weight-to-weight basis (aspect 10).

In aspects, the invention provides a composition of any one or more of aspects 1-10, wherein the release retarding agent comprises at least two distinct coats, at least one coat being at least generally composed of a distinct polymer composition from at least one other coat of the release retarding agent (aspect 11).

In aspects, the invention provides a composition of any one or more of aspects 1-11, wherein the release retarding agent comprises a first coat that is at least mostly composed of a hydrophilic polymer (aspect 12). 12, wherein the release retarding agent comprises a second coat that is at least mostly composed of a hydrophobic polymer (aspect 13).

In aspects, the invention provides a composition of any one or more of aspects 1-13, wherein (a) the release retarding agent comprises a first coat that is at least mostly composed of a hydrophilic polymer and (b) the release retarding agent comprises a second coat that is at least mostly composed of a hydrophobic polymer (aspect 14).

In aspects, the invention provides a composition of any one or more of aspects 1-14, wherein the second coat comprises a mixture of a hydrophobic polymer and a hydrophilic polymer (aspect 15).

In aspects, the invention provides a composition of any one or more of aspects 1-15, wherein the hydrophilic polymer is at least mostly (if not generally, substantially, essentially, or only) composed of a hydrophilic cellulosic polymer (aspect 16).

In aspects, the invention provides a composition of any one or more of aspects 1-16, wherein the hydrophilic cellulosic polymer is at least mostly (if not generally, substantially, essentially, or only) composed of hydroxypropyl methylcellulose (HPMC) (aspects 17).

In aspects, the invention provides a composition of any one or more of aspects 1-17, wherein the hydrophobic polymer is at least mostly (if not generally, substantially, essentially, or only) composed of a hydrophobic cellulosic polymer (aspect 18).

In aspects, the invention provides a composition of any one or more of aspects 1-18, wherein the hydrophobic cellulosic polymer is at least mostly (if not generally, substantially, essentially, or only) composed of ethyl cellulose (aspect 19).

In aspects, the invention provides a composition of any one or more of aspects 1-19, wherein the mixture comprises a mixture comprising mostly ethyl cellulose and further comprising HPMC (aspect 20).

In aspects, the invention provides a composition of any one or more of aspects 1-20, wherein the ratio of ethyl cellulose to HPMC is about 3:1 to about 8:1, such as 4:1 to 6:1, such as about 5:1 (aspect 21).

In aspects, the invention provides a composition of any one or more of aspects 1-21, wherein the drug-resin complex makes up about 30-50% of the composition on a weight-by-weight basis (aspect 22). 22, wherein the suspension base comprises a starch that makes up about 5-15% of the composition on a w/w basis (aspect 23).

In aspects, the invention provides a composition of any one or more of aspects 1-23, wherein the suspension base comprises a disaccharide, wherein the disaccharide makes up about 7.5%-15% of the composition on a w/w basis (aspect 24).

In aspects, the invention provides a composition of any one or more of aspects 1-24, wherein the disaccharide comprises a mixture of sucrose and sucralose (aspect 25).

In aspects, the invention provides a composition of any one or more of aspects 1-25, wherein the suspension base comprises a thickening agent that makes up at least about 3% of the composition on a w/w basis, as about 2.5-7.5%, such as about 5% (aspect 26).

In aspects, the invention provides a composition of any one or more of aspects 1-26, wherein the thickening agent is at least mostly (or generally, substantially, essentially, or only) composed of hydroxypropyl cellulose (HPC) (aspect 27).

In aspects, the invention provides a composition of any one or more of aspects 1-27, wherein the composition is adapted for oral administration (aspect 28).

In aspects, the invention provides a composition of any one or more of aspects 1-28, wherein the composition is in the form of a capsule (aspect 29).

In aspects, the invention provides a composition of any one or more of aspects 1-29, wherein the composition is in the form of a tablet (aspect 30).

In aspects, the invention provides a composition of any one or more of aspects 1-30, wherein the composition comprises the drug-resin complex in a concentration of about 50-about 100 mg/mL (aspect 31).

In aspects, the invention provides a composition of any one or more of aspects 1-31, wherein the composition is in a unit dosage form for a single administration to a patient (aspect 32).

In aspects, the invention provides a composition of any one or more of aspects 1-32, wherein the composition comprises an amount expected to be effective as a once-a-day treatment for myotonia (aspect 33). 33, wherein the composition comprises about 100-about 600 mg of mexiletine hydrochloride (aspect 34).

In aspects, the invention provides a composition of any one or more of aspects 1-34, wherein the composition comprises ˜100-400 mg of mexiletine hydrochloride (aspect 35).

In aspects, the invention provides a composition of any one or more of aspects 1-35 wherein the pharmaceutical composition exhibits an in vitro dissolution profile such that after 0-2 hours, from about 0% to about 30% by weight of mexiletine is released, after 0-4 hours, from about 10% to about 40% by weight of mexiletine is released, after 4-12 hours, from about 30% to about 70% by weight of mexiletine is released, from 6-14 hours, more than about 60% by weight of mexiletine is released and, more than about 80% by weight of mexiletine is released up to 24 hours when measured using the USP type IV apparatus having flow rate 8 ml/min along with 6 gm glass beads in 0.1N HCl media, 37° C.±0.5° C. (aspect 36).

In aspects, the invention provides a composition of any one or more of aspects 1-36, wherein the pharmaceutical composition exhibits an in vitro dissolution profile such that after 0-2 hours, from about 0% to about 25% by weight of mexiletine is released, after 0-4 hours, from about 10% to about 40% by weight of mexiletine is released, after 4-12 hours, from about 30% to about 65% or more than 65% by weight of mexiletine is released, when measured using the USP type IV apparatus having flow rate 8 ml/min along with 6 gm glass beads in pH 5.0 acetate buffer media, 37° C.±0.5° C. (aspects 37).

In aspects, the invention provides a composition of any one or more of aspects 1-37, wherein the pharmaceutical composition exhibits an in vitro dissolution profile such that after 0-2 hours, from about 0% to about 35% by weight of mexiletine is released, after 0-4 hours, from about 10% to about 50% by weight of mexiletine is released, after 4-12 hours, from about 30% to about 75% or more than 75% by weight of mexiletine is released, when measured using the USP type IV apparatus having flow rate 8 ml/min along with 6 gm glass beads in pH 6.8 phosphate buffer media, 37° C.±0.5° C. (aspect 38).

In aspects, the invention provides a composition according to any one or more of aspects 1-38, wherein the pharmaceutical composition releases not more than 50% by weight of mexiletine in an initial 1 hour in 500 mL 0.1N HCl and followed by 700 mL pH 4.5 acetate buffer changeover media the said composition releases not less than 65% by weight of mexiletine in 2 hours and further followed by 900 mL pH 6.8 phosphate buffer changeover media the said composition releases not less than 85% by weight of mexiletine in 4 hours when measured in United States Pharmacopoeia (USP) type II dissolution apparatus, rotated at 75 rpm at a temperature of 370±0.5° C. (aspect 39).

In aspects, the invention provides a composition according to any one or more of aspects 1-39, wherein upon a single oral administration of the composition (1) Tₘₐₓ occurs between about 5 to about 8 hours after administration in most human patients and (2) the plasma concentration of mexiletine in most humans 18 hours after administration is at least about 33% Cₘₐₓ, such as at least about 40% of Cₘₐₓ (aspect 40).

In aspects, the invention provides a composition of any one or more of aspects 1-40, wherein the plasma concentration in most patients receiving a single oral administration of the composition remains within about 20% of Cₘₐₓ for a period of at least ˜4 hours (aspect 41).

In aspects, the invention provides a composition of any one or more of aspects 1-41, wherein the plasma concentration in most patients receiving a single oral administration of the composition remains within about 15% of Cₘₐₓ for a period of at least ˜4 hours (aspect 42).

In aspects, the invention provides a composition of any one or more of aspects 1-42, wherein the plasma concentration in most patients receiving a single oral administration of the composition remains within about 10% of Cₘₐₓ for a period of at least ˜4 hours (aspect 43).

In aspects, the invention provides a composition of any one or more of aspects 1-43, wherein the plasma concentration in most patients receiving a single oral administration of the composition remains within about 20% of Cₘₐₓ for a period of at least ˜5 hours (aspect 44).

In aspects, the invention provides a composition of any one or more of aspects 1-44, wherein the plasma concentration in most patients receiving a single oral administration of the composition remains within about 15% of Cₘₐₓ for a period of at least ˜5 hours (aspect 45).

In aspects, the invention provides a composition of any one or more of aspects 1-45, wherein the plasma concentration in most patients receiving a single oral administration of the composition remains within about 10% of Cₘₐₓ for a period of at least ˜5 hours (aspect 46).

In aspects, the invention provides a composition of any one or more of aspects 1-46, wherein the plasma concentration in most patients receiving a single oral administration of the composition remains within about 20% of Cₘₐₓ for a period of at least ˜6 hours (aspect 47). 47, wherein the plasma concentration in most patients receiving a single oral administration of the composition remains within about 15% of Cₘₐₓ for a period of at least ˜6 hours (aspect 48).

In aspects, the invention provides a composition of any one or more of aspects 1-48, wherein the plasma concentration in most patients receiving a single oral administration of the composition remains within about 10% of Cₘₐₓ for a period of at least ˜6 hours (aspect 49).

In aspects, the invention provides a composition of any one or more of aspects 1-49, wherein the plasma concentration of mexiletine in most human patients receiving a single oral administration of the compound at either 3 hours after administration, 4 hours after administration, or both, is within about 25% of the plasma concentration of mexiletine 18 hours after administration, 20 hours after administration, or both (aspect 50).

In aspects, the invention provides a composition of any one or more of aspects 1-50, wherein the composition exhibits at least one of the following pharmacokinetic profile characteristics:
(a) median, single dose, Tmax about 5 to about 8 hours;
(b) mean, single dose, Cmax about 700-1250 ng/mL, such as about 700-1200 or 725-1200 ng/mL, e.g., 763.2237 ng/mL to about 1162.5969 ng/mL;
(c) mean, single dose, AUC0-24 about 12500 ng*hr/mL, such as 12750 ng*hr/mL (e.g., 12826.7066 ng*hr/m) to about 16000 ng*hr/mL, such as 16250 ng*hr/mL (e.g., 16323.9271 ng*hr/mL);
(d) mean, single dose, AUC0-t of about 21000-25000 ng*hr/mL, such as about 22000-24000 ng*hr/mL (e.g., about 22301.7312 ng*hr/mL to about 23909.8110 ng*hr/mL);
(e) mean, single dose, AUC0-∞ about 22500-26000 ng*hr/mL, such as about 23000-25000 ng*hr/mL (e.g., about 23345.7516 ng*hr/mL to about 25363.6969 ng*hr/mL); and (f) mean, single dose, t½ about 10 hours to about 13.5 hours (aspect 51).

In aspects, the invention provides a composition of any one or more of aspects 1-51, wherein the composition exhibits a mean, single dose, t½ about 10 hours to about 13.5 hours (aspect 52).

In aspects, the invention provides a composition of any one or more of aspects 1-52, wherein the composition exhibits a median, single dose, Tₘₐₓ˜5 to ˜8 hours (aspects 53). 53, wherein the composition exhibits a median, single dose, Tₘₐₓ-6 or more hours (aspects 54).

In aspects, the invention provides a composition of any one or more of aspects 1-54, wherein the composition exhibits a mean, single dose, Cₘₐₓ of about 700 ng/mL or more, such as about 750 ng/mL or more, such as about 763.2237 ng/mL or more (aspects 55).

In aspects, the invention provides a composition of any one or more of aspects 1-55, wherein the composition exhibits a mean, single dose, AUC₀₋₂₄ of about 12000-13500 ng*hr/mL, such as about 12500-13000 ng*hr/mL, such as ˜12826.7066 ng*hr/mL (aspect 56).

In aspects, the invention provides a composition of any one or more of aspects 1-56, wherein the composition exhibits a mean, single dose, AUC₀₋ₜ of about 21500-24000 ng*hr/mL, such as about 22000-23000 ng*hr/mL, such as ˜22301.7312 ng*hr/mL (aspect 57).

In aspects, the invention provides a composition of any one or more of aspects 1-57, wherein the composition exhibits a mean, single dose, AUC_{0-∞} of about 22500-24000 ng*hr/mL, such as about 23000-23500 ng*hr/mL, such as ˜23345.7516 ng*hr/mL (aspect 58).

In aspects, the invention provides a composition of any one or more of aspects 1-58, wherein the composition exhibits a mean, single dose, t_{1/2}˜12 hours or more (aspect 59).

In aspects, the invention provides a composition of any one or more of aspects 1-59, wherein the pharmaceutical composition exhibits a detectable or significant increase in mean Cₘₐₓ, mean AUC, or both, in a fed condition with respect to fasting condition (aspect 60).

In aspects, the invention provides a composition of any one or more of aspects 1-60, which has a mean fed/fasted ratio of AUC_{0-∞} of ˜1.13 or more (aspect 61).

In aspects, the invention provides a composition of any one or more of aspects 1-61, wherein the composition has a mean fed/fasted ratio of Cₘₐₓ of ˜1.25 or more (aspect 62).

In aspects, the invention provides a pharmaceutical composition comprising a therapeutically effective amount of mexiletine or pharmaceutically acceptable salt thereof and a release retarding agent, wherein the pharmaceutical composition exhibits an in vitro dissolution profile such that after 0-2 hours, from about 0% to about 30% by weight of mexiletine is released, after 0-4 hours, from about 10% to about 40% by weight of mexiletine is released, after 4-12 hours, from about 30% to about 70% by weight of mexiletine is released, from 6-14 hours, more than about 60% by weight of mexiletine is released and, more than about 80% by weight of mexiletine is released up to 24 hours when measured using the USP type IV apparatus having flow rate 8 ml/min along with 6 gm glass beads in 0.1N HCl media, 37° C.±0.5° C. (aspect 63).

In aspects, the invention provides a pharmaceutical composition comprising a therapeutically effective amount of mexiletine or pharmaceutically acceptable salt thereof and a release retarding agent, wherein the pharmaceutical composition exhibits an in vitro dissolution profile such that after 0-2 hours, from about 0% to about 25% by weight of mexiletine is released, after 0-4 hours, from about 10% to about 40% by weight of mexiletine is released, after 4-12 hours, from about 30% to about 65% or more than 65% by weight of mexiletine is released, when measured using the USP type IV apparatus having flow rate 8 ml/min along with 6 gm glass beads in pH 5.0 acetate buffer media, 37° C.±0.5° C. (aspect 64).

In aspects, the invention provides a pharmaceutical composition comprising a therapeutically effective amount of mexiletine or a pharmaceutically acceptable salt thereof and a release retarding agent, wherein the pharmaceutical composition exhibits an in vitro dissolution profile such that after 0-2 hours, from about 0% to about 35% by weight of mexiletine is released, after 0-4 hours, from about 10% to about 50% by weight of mexiletine is released, after 4-12 hours, from about 30% to about 75% or more than 75% by weight of mexiletine is released, when measured using the USP type IV apparatus having flow rate 8 ml/min along with 6 gm glass beads in pH 6.8 phosphate buffer media, 37° C.±0.5° C. (aspect 65).

In aspects, the invention provides a pharmaceutical composition comprising a therapeutically effective amount of mexiletine or a pharmaceutically acceptable salt thereof and a release retarding agent, wherein the pharmaceutical composition releases not more than 50% by weight of mexiletine in an initial 1 hour in 500 mL 0.1N HCl and followed by 700 mL pH 4.5 acetate buffer changeover media the said composition releases not less than 65% by weight of mexiletine in 2 hours and further followed by 900 mL pH 6.8 phosphate buffer changeover media the said composition releases not less than 85% by weight of mexiletine in 4 hours when measured in United States Pharmacopoeia (USP) type II dissolution apparatus, rotated at 75 rpm at a temperature of 370±0.5° C. (aspect 66).

In aspects, the invention provides a composition according to any one or more of aspects 1-66, wherein the retarding agent comprises a pharmaceutically acceptable ion exchange resin (aspect 67). 68, wherein the ion exchange resin is a cationic exchange resin (aspect 68).

In aspects, the invention provides a composition of any one or more of aspects 1-68, wherein the cation exchange resin is at least mostly composed of a material having a pKa of about −1.5 or less (aspect 69).

In aspects, the invention provides a composition of any one or more of aspects 1-69, wherein at least generally all of the cation exchange resin comprises a sulfonate group (aspect 70).

In aspects, the invention provides a composition of any one or more of aspects 1-70, wherein at least generally all of the cation exchange resin comprises a polystyrene component (aspect 71).

In aspects, the invention provides a composition of any one or more of aspects 1-71, wherein the cation exchange resin makes up about 30% to about 85% of the drug-resin complex on a weight-to-weight basis (aspect 72).

In aspects, the invention provides a composition of any one or more of aspects 1-72, wherein at least generally all of the cation exchange resin is composed of sodium polystyrene sulfonate (aspect 73).

In aspects, the invention provides a composition of any one or more of aspects 1-73, wherein the ion exchange resin is present in an amount such that the mexiletine and ion exchange resin are present in a ratio of about 1:0.5 to about 1:5 (aspect 74).

In aspects, the invention provides a composition of any one or more of aspects 1-74, wherein the mexiletine is present as a pharmaceutically acceptable salt of mexiletine (aspect 75).

In aspects, the invention provides a composition of any one or more of aspects 1-75, wherein the pharmaceutically acceptable salt of mexiletine and the ion exchange resin form a pharmaceutically acceptable drug-resin complex (aspect 76).

In aspects, the invention provides a composition of any one or more of aspects 1-76, wherein the retarding agent comprises a hydrophilic release retardant polymer, a hydrophobic release retardant polymer, or both, wherein the hydrophilic polymer, hydrophobic polymer, or both, form coated complex particles with the drug-resin complex and detectably delay the release of mexiletine from the coated complex particles (aspect 77). 77, wherein the composition comprises an amount of an excipient that is effective to detectably change one or more properties of the composition other than the pharmaceutical activity of the composition (aspect 78).

In aspects, the invention provides a composition of any one or more of aspects 1-78, wherein the properties associated with the excipient comprise the taste of the formulation, the stability of the formulation, the viscosity of the formulation, or a combination of any or all thereof (aspect 79).

In aspects, the invention provides a composition of any one or more of aspects 1-79, wherein the composition further comprises a suspension base that suspends coated complex particles to form a controlled release liquid suspension of mexiletine (aspect 80).

In aspects, the invention provides a composition of any one or more of aspects 1-80, wherein the mexiletine makes up about 15% to about 70% of the drug-resin complex of the composition on a weight-to-weight basis (aspect 81).

In aspects, the invention provides a composition of any one or more of aspects 1-81, wherein the release retarding agent comprises at least two distinct coats, at least one coat being at least generally composed of a distinct polymer composition from at least one other coat of the release retarding agent (aspect 82).

In aspects, the invention provides a composition of any one or more of aspects 1-82, wherein the release retarding agent comprises a first coat that is at least mostly composed of a hydrophilic polymer (aspect 83).

In aspects, the invention provides a composition of any one or more of aspects 1-83, wherein the release retarding agent comprises a second coat that is at least mostly composed of a hydrophobic polymer (aspect 84).

In aspects, the invention provides a composition of any one more of aspects 1-84, wherein (a) the release retarding agent comprises a first coat that is at least mostly composed of a hydrophilic polymer and (b) the release retarding agent comprises a second coat that is at least mostly composed of a hydrophobic polymer (aspect 85).

In aspects, the invention provides a composition of any one or more of aspects 1-85, wherein the second coat comprises a mixture of a hydrophobic polymer and a hydrophilic polymer (aspect 86). 86, wherein the hydrophilic polymer is at least mostly composed of a hydrophilic cellulosic polymer (aspect 87).

In aspects, the invention provides a composition of any one or more of aspects 1-87, wherein the hydrophilic cellulosic polymer is at least mostly composed of hydroxypropyl methylcellulose (HPMC) (aspect 88).

In aspects, the invention provides a composition of any one or more of aspects 1-88, wherein the hydrophobic polymer is at least mostly composed of a hydrophobic cellulosic polymer (aspect 89).

In aspects, the invention provides a composition of any one or more of aspects 1-89, wherein the hydrophobic cellulosic polymer is at least mostly composed of ethyl cellulose (aspect 90).

In aspects, the invention provides a composition of any one or more of aspects 1-90, wherein the second coat comprises a mixture comprising mostly ethyl cellulose and further comprising HPMC (aspect 91).

In aspects, the invention provides a composition of any one or more of aspects 1-91, wherein the ratio of ethyl cellulose to HPMC is about 3:1 to about 8:1, such as 4:1 to 6:1, such as about 5:1 (aspect 92).

In aspects, the invention provides a composition of any one or more of aspects 1-92, wherein the drug-resin complex makes up about 30-50% of the composition on a weight-by-weight basis (aspect 93).

In aspects, the invention provides a composition of any one or more of aspects 1-93, wherein the suspension base comprises a starch that makes up about 5-15% of the composition on a w/w basis (aspect 94).

In aspects, the invention provides a composition of any one or more of aspects 1-93, wherein the suspension base comprises a disaccharide, wherein the disaccharide makes up about 7.5%-15% of the composition on a w/w basis (aspect 95).

In aspects, the invention provides a composition of any one or more of aspects 1-95, wherein the disaccharide comprises a mixture of sucrose and sucralose (aspect 96). 96, wherein the suspension base comprises a thickening agent that makes up at least about 3% of the composition on a w/w basis, as about 2.5-7.5%, such as about 5% (aspect 97).

In aspects, the invention provides a composition of any one or more of aspects 1-97, wherein the thickening agent is at least mostly (if not generally only, substantially only, or only) composed of hydroxypropyl cellulose (HPC) (aspect 98).

In aspects, the invention provides a composition of any one or more of aspects 1-98, wherein the composition is adapted for oral administration (aspect 99).

In aspects, the invention provides a composition of any one or more of aspects 1-99, wherein the composition is in the form of a capsule (aspect 100).

In aspects, the invention provides a composition of any one or more of aspects 1-100 wherein the composition is in the form of a tablet (aspect 101).

In aspects, the invention provides a composition of any one or more of aspects 1-101, wherein the composition comprises the drug-resin complex in a concentration of about 50-about 100 mg/mL (aspect 102).

In aspects, the invention provides a composition of any one or more of aspects 1-102, wherein the composition is in a unit dosage form for a single administration to a patient (aspect 103).

In aspects, the invention provides a composition of any one or more of aspects 1-103, wherein the composition comprises an amount expected to be effective as a once-a-day treatment for myotonia (aspect 104).

In aspects, the invention provides a composition of any one or more of aspects 1-104, wherein the composition comprises about 100-about 600 mg of mexiletine hydrochloride (aspect 105).

In aspects, the invention provides a composition of any one or more of aspects 1-105, wherein the composition comprises ˜100-400 mg of mexiletine hydrochloride (aspect 106).

In aspects, the invention provides a composition according to any one or more of aspects 1-106, wherein the composition is stable, as evidenced by a loss of less than 10% (e.g., less than 5%, less than 2%, or less than 1%) of starting/initial mexiletine in the composition, under either standard US FDA accelerated stability test conditions (40° C./75% RH) for a period of at least 2 months, such as at least 3 months or at least 6 months, or ordinary (long term) stability testing test conditions for a period of at least 12 months, such as at least 18 months or 24 months (aspect 107).

In aspects, the invention provides a composition of any one or more of aspects 1-107, wherein upon a single administration of an effective dose of the composition to a human recipient (1) Tₘₐₓ occurs between about 5 to about 8 hours after administration in most human patients and (2) the plasma concentration of mexiletine in most humans 18 hours after administration is at least about 33% Cₘₐₓ, such as at least about 40% of Cₘₐₓ (aspect 108).

In aspects, the invention provides a composition of any one or more of aspects 1-108, wherein the plasma concentration in most patients receiving a single oral administration of the composition remains within about 20% of Cₘₐₓ for a period of at least ˜4 hours (aspect 109).

In aspects, the invention provides a composition of any one or more of aspects 1-109, wherein the plasma concentration in most patients receiving a single oral administration of the composition remains within about 15% of Cₘₐₓ for a period of at least ˜4 hours (aspect 110).

In aspects, the invention provides a composition of any one or more of aspects 1-110, wherein the plasma concentration in most patients receiving a single oral administration of the composition remains within about 10% of Cₘₐₓ for a period of at least ˜4 hours (aspect 111).

In aspects, the invention provides a composition of any one or more of aspects 1-111, wherein the plasma concentration in most patients receiving a single oral administration of the composition remains within about 20% of Cₘₐₓ for a period of at least ˜5 hours (aspect 112).

In aspects, the invention provides a composition of any one or more of aspects 1-112, wherein the plasma concentration in most patients receiving a single oral administration of the composition remains within about 15% of Cₘₐₓ for a period of at least ˜5 hours (aspect 113).

In aspects, the invention provides a composition of any one or more of aspects 1-113, wherein the plasma concentration in most patients receiving a single oral administration of the composition remains within about 10% of Cₘₐₓ for a period of at least ˜5 hours (aspect 114).

In aspects, the invention provides a composition of any one or more of aspects 1-114, wherein the plasma concentration in most patients receiving a single oral administration of the composition remains within about 20% of Cₘₐₓ for a period of at least ˜6 hours (aspect 115).

In aspects, the invention provides a composition of any one or more of aspects 1-115, wherein the plasma concentration in most patients receiving a single oral administration of the composition remains within about 15% of Cₘₐₓ for a period of at least ˜6 hours (aspect 116). 116, wherein the plasma concentration in most patients receiving a single oral administration of the composition remains within about 10% of Cₘₐₓ for at least about 6 hours (aspect 117).

In aspects, the invention provides a composition of any one or more of aspects 1-117, wherein the plasma concentration of mexiletine in most human patients receiving a single oral administration of the compound at either 3 hours after administration, 4 hours after administration, or both, is within about 25% of the plasma concentration of mexiletine 18 hours after administration (aspect 118).

In aspects, the invention provides a pharmaceutical composition comprising a therapeutically effective amount of mexiletine or pharmaceutically acceptable salt thereof and an effective amount of a release retarding agent contained in an oral delivery formulation that exhibits a delayed release profile such that upon a single oral administration of the composition (1) Tₘₐₓ occurs between about 5 to about 8 hours after administration in most human patients and (2) the plasma concentration of mexiletine in most humans 18 hours after administration is at least about 35% of Cₘₐₓ, such as at least about 40% of Cₘₐₓ (aspect 119).

In aspects, the invention provides a composition of any one or more of aspects 1-119, wherein the plasma concentration in most patients receiving a single oral administration of the composition remains within about 20% of Cₘₐₓ for at least about 4 hours (aspect 120).

In aspects, the invention provides a composition of any one or more of aspects 1-120, wherein the plasma concentration in most patients receiving a single oral administration of the composition remains within about 15% of Cₘₐₓ for at least ˜t 4 hours (aspect 121).

In aspects, the invention provides a composition of any one or more of aspects 1-121, wherein the plasma concentration in most patients receiving a single oral administration of the composition remains within about 10% of Cₘₐₓ for a period of at least ˜4 hours (aspect 122).

In aspects, the invention provides a composition of any one or more of aspects 1-122, wherein the plasma concentration in most patients receiving a single oral administration of the composition remains within about 20% of Cₘₐₓ for a period of at least ˜5 hours (aspect 123).

In aspects, the invention provides a composition of any one or more of aspects 1-123, wherein the plasma concentration in most patients receiving a single oral administration of the composition remains within about 15% of Cₘₐₓ for a period of at least ˜5 hours (aspect 124). 124, wherein the plasma concentration in most patients receiving a single oral administration of the composition remains within about 10% of Cₘₐₓ for a period of at least ˜5 hours (aspect 125).

In aspects, the invention provides a composition of any one or more of aspects 1-125, wherein the plasma concentration in most patients receiving a single oral administration of the composition remains within about 20% of Cₘₐₓ for a period of at least ˜6 hours (aspect 126).

In aspects, the invention provides the composition of aspect 126, wherein the plasma concentration in most patients receiving a single oral administration of the composition remains within about 15% of Cₘₐₓ for a period of at least about 6 hours (aspect 127).

In aspects, the invention provides the composition of aspect 127, wherein the plasma concentration in most patients receiving a single oral administration of the composition remains within about 10% of Cₘₐₓ for a period of at least about 6 hours (aspect 128).

In aspects, the invention provides the composition of any one or more of aspects 120-128, wherein the plasma concentration of mexiletine in most human patients receiving a single oral administration of the compound at either 3 hours after administration, 4 hours after administration, or both, is within about 25% of the plasma concentration of mexiletine 18 hours after administration (aspect 129).

In aspects, the invention provides the composition of any one or more of aspects 120-129, wherein the composition incorporates one or more of the features of the compositions according to any one or more of aspects 1-62 (aspect 130).

In aspects, the invention provides a pharmaceutical composition comprising mexiletine or a pharmaceutically acceptable salt thereof and a release retarding agent, wherein the pharmaceutical composition exhibits at least one of the following pharmacokinetic profile characteristics:
(a) median, single dose, Tmax about 5 to about 8 hours;
(b) mean, single dose, Cmax about 700-1250 ng/mL, such as from about 700-1200 or 725-1200 ng/mL, e.g., about 763.2237 ng/mL to about 1100-1200 ng/mL, e.g., about 1125-1175 ng/mL, such as about 1162.5969 ng/mL;
(c) mean, single dose, AUC0-24 from about 12500-13000 ng*hr/mL, such as about 12750 ng*hr/mL (e.g., about 12826.7066 ng*hr/m) to about 16000-17000 ng*hr/mL, such as 16500 ng*hr/mL (e.g., about 16323.9271 ng*hr/mL);
(d) mean, single dose, AUC0-t of about 21000-25000 ng*hr/mL, such as about 22000-24250 ng*hr/mL (e.g., about 22301.7312 ng*hr/mL to about 23909.8110 ng*hr/mL);
(e) mean, single dose, AUC0-∞ of about 22500-26000 ng*hr/mL, such as about 23000-25500 ng*hr/mL (e.g., from about 23345.7516 ng*hr/mL to about 25363.6969 ng*hr/mL); and
(f) mean, single dose, t½ about 10 hours to about 13.5 hours (aspect 131).

In aspects, the invention provides the composition of aspect 131, wherein the composition exhibits a mean, single dose, t_{1/2} about 10 hours to about 13.5 hours (aspect 132).

In aspects, the invention provides a composition of aspect 131 or aspect 132, wherein the composition exhibits a median, single dose, Tₘₐₓ about 5 to ˜8 hours (aspect 133).

In aspects, the invention provides a composition of aspect 133, wherein the composition exhibits a median, single dose, Tₘₐₓ about 6 or more hours (aspect 134).

In aspects, the invention provides the composition of any one or more of aspects 131-134, wherein the composition exhibits a mean, single dose, Cₘₐₓ of about 700 ng/mL or more, such as about 750 ng/mL or more, such as about 763.2237 ng/mL or more (aspect 135).

In aspects, the invention provides the composition of any one or more of aspects 131-135, wherein the composition exhibits a mean, single dose, AUC₀₋₂₄ of about 12000-13500 ng*hr/mL, such as about 12500-13000 ng*hr/mL, such as ˜12826.7066 ng*hr/mL (aspect 136).

In aspects, the invention provides the composition of any one or more of aspects 131-136, wherein the composition exhibits a mean, single dose, AUC₀₋ₜ of ˜21500-23500 ng*hr/mL, such as ˜22000-22750 ng*hr/mL, such as about 22301.7312 ng*hr/mL (aspect 137).

In aspects, the invention provides the composition of any one or more of aspects 131-137, wherein the composition exhibits a mean, single dose, AUC_{0-∞} of about 22500-24500 ng*hr/mL, such as about 23000-23750 ng*hr/mL, such as ˜23345.7516 ng*hr/mL (aspect 138).

In aspects, the invention provides the composition of any one or more of aspects 131-138, wherein the composition exhibits a mean, single dose, t_{1/2} about 12 hours or more (aspect 139).

In aspects, the invention provides a pharmaceutical composition comprising a therapeutically effective amount of mexiletine or a pharmaceutically acceptable salt thereof and a release retarding agent, wherein the pharmaceutical composition exhibits a detectable or significant increase in mean Cₘₐₓ, mean AUC, or both, in a fed condition with respect to fasting condition (aspect 140).

In aspects, the invention provides the composition of aspect 140, wherein the composition has a mean fed/fasted ratio of AUC_{0-∞} of about 1.13 or more (aspect 141).

In aspects, the invention provides the composition of aspect 141, wherein the composition has a mean fed/fasted ratio of Cₘₐₓ of about 1.25 or more (aspect 142).

In aspects, the invention provides the composition of any one or more of aspects 1-62, wherein the composition exhibits the pharmacokinetic profile characteristics of aspect 131 (aspect 143).

In aspects, the invention provides a pharmaceutically acceptable composition comprising a therapeutically effective amount of mexiletine and a retarding agent, wherein the composition exhibits a plasma level of mexiletine and three or more time points that is statistically similar to the plasma level of mexiletine achieved by a corresponding single administration of Test Product 1 (T1) at three or more time points between 3 and 24 hours after administration under the same conditions (aspect 144).

In aspects, the invention provides a pharmaceutically acceptable composition comprising a therapeutically effective amount of mexiletine and a retarding agent, wherein the composition exhibits a plasma level of mexiletine and five or more time points that is statistically similar to the plasma level of mexiletine achieved by a corresponding single administration of Test Product 1 (T1) at five or more time points between 3 and 24 hours after administration under the same conditions (aspect 145).

In aspects, the invention provides a pharmaceutically acceptable composition comprising a therapeutically effective amount of mexiletine and a retarding agent, wherein the composition exhibits a plasma level of mexiletine and seven or more time points that is statistically similar to the plasma level of mexiletine achieved by a corresponding single administration of Test Product 1 (T1) at seven or more time points between 3 and 24 hours after administration under the same conditions (aspect 146).

In aspects, the invention provides a pharmaceutically acceptable composition comprising a therapeutically effective amount of mexiletine and a retarding agent, wherein the composition exhibits a plasma level of mexiletine and three or more time points that is about the same the plasma level of mexiletine achieved by a corresponding single administration of Test Product 2 (T2) at three or more time points between 3 and 24 hours after administration under the same conditions (aspect 147).

In aspects, the invention provides a pharmaceutically acceptable composition comprising a therapeutically effective amount of mexiletine and a retarding agent, wherein the composition exhibits a plasma level of mexiletine and five or more time points that is about the same the plasma level of mexiletine achieved by a corresponding single administration of Test Product 2 (T2) at five or more time points between 3 and 24 hours after administration under the same conditions (aspect 148).

In aspects, the invention provides a pharmaceutically acceptable composition comprising a therapeutically effective amount of mexiletine and a retarding agent, wherein the composition exhibits a plasma level of mexiletine and seven or more time points that is about the same the plasma level of mexiletine achieved by a corresponding single administration of Test Product 2 (T2) at seven or more time points between 3 and 24 hours after administration under the same conditions (aspect 149).

In aspects, the invention provides a pharmaceutical composition comprising (a) a therapeutically effective amount of a pharmaceutically acceptable salt of mexiletine and means for retarding release of the mexiletine such that the composition exhibits a Tₘₐₓ of about 5-8 hours and a t_{1/2} of about 10 hours to about 13.5 hours (aspect 150).

In aspects, the invention provides the composition of aspect 150, wherein the composition comprises means for delivering the composition to a patient via oral administration (aspect 151).

In aspects, the invention provides the composition of aspect 151, wherein the composition comprises means for suspending the mexiletine in a liquid formulation (aspect 152).

In aspects, the invention provides the composition of any one or more of aspects 150-152, wherein the composition exhibits the pharmacokinetic profile characteristics of any one or more of aspects 131-149 (aspect 153).

In aspects, the invention provides the composition of any one or more of aspects 150-152, wherein the composition exhibits the dissolution properties of any one or more of aspects 63-117 (aspect 154).

In aspects, the invention provides a product made by the process comprising (1) providing a pharmaceutically acceptable form of mexiletine or a pharmaceutically acceptable salt thereof; (2) blending the mexiletine with an ion exchange resin to form a pharmaceutically acceptable ion exchange resin; (3) dissolving and/or dispersing a release retarding agent and one or more pharmaceutically acceptable coating additives in a suitable solvent to prepare a coating composition; and (4) applying the coating composition onto the complex of mexiletine and ion exchange resin to obtain a controlled release once daily composition of mexiletine or pharmaceutically acceptable salt thereof (aspect 155).

In aspects, the invention provides the product of aspect 155, wherein the pharmaceutically acceptable ion exchange resin comprises two layers, a hydrophilic and hydrophobic release retardant polymer (aspect 156).

In aspects, the invention provides the product of aspect 156, wherein the mexiletine or pharmaceutically acceptable salt thereof comprises mexiletine hydrochloride (aspect 157).

In aspects, the invention provides a product produced by the process described in any one of the examples described herein (aspect 158).

In aspects, the invention provides a product produced by a process comprising most, generally all, or all of the methods of production or step(s) thereof described in the examples herein (aspect 159).

In aspects, the invention provides the composition of any one of aspects 1-62, wherein the composition is made by a process of manufacturing wherein ion exchange resin particles are loaded with pharmaceutical drugs prior to incorporation in a polymeric system to form a drug resin complex, which is further coated by release retarding polymer to form a controlled release drug resin complex (aspect 160).

In aspects, the invention provides a method of treating myotonia in a patient in need thereof comprising administering to the patient an effective amount of a composition or product (as applicable) according to any one or more of aspects 1-aspect 160 (aspect 161).

In aspects, the invention provides the method of aspect 161, wherein the method comprises administering a dose of the composition or product (as applicable) to the patient once a day for part or all of the treatment period, twice a day for part or all of the treatment period, or both (aspect 162).

In aspects, the invention provides the method of aspect 162, wherein the method comprises mostly administering one dose of the composition or product (as applicable) per day (aspect 163).

In aspects, the invention provides the method of aspect 163, wherein the method is as effective as three times a day administration of Namuscla in a significant number of patients (aspect 164).

In aspects, the invention provides the method of aspect 164, wherein the method is at least as effective as three times a day administration of Namuscla in most patients (aspect 165).

In aspects, the invention provides the method of aspect 161 or aspect 162, wherein the method is associated with less gastrointestinal adverse results than that associated with the use of Namuscla (aspect 166).

In aspects, the invention provides the method of any one or more of aspects 161-163, wherein a significantly greater number of patients receiving the treatment continue with the treatment over an extended period (e.g., at least about 1 month, at least about 3 months, at least about 6 months, at least about 1 year) as compared to treatment with Namuscla (aspect 167).

In aspects, the invention provides the method of any one of aspects 161-164, wherein the method comprises the patient taking the product after eating (aspect 168).

In aspects, the invention provides the method of any one or more of aspects 161-168, wherein the treatment is administered via the means of any one or more of aspects 150-154 (aspect 169).

In aspects, the invention provides a method for producing a pharmaceutically acceptable mexiletine composition comprising steps (1) providing a pharmaceutically acceptable form of mexiletine or a pharmaceutically acceptable salt thereof; (2) blending the mexiletine with an ion exchange resin to form a pharmaceutically acceptable ion exchange resin; (3) dissolving and/or dispersing a release retarding agent and one or more pharmaceutically acceptable coating additives in a suitable solvent to prepare a coating composition; and (4) applying the coating composition onto the complex of mexiletine and ion exchange resin to obtain a controlled release once daily composition of mexiletine or pharmaceutically acceptable salt thereof (aspect 170).

In aspects, the invention provides a method for producing the composition of any one or more of aspects 1-62, wherein the ion exchange resin particles are loaded with pharmaceutical drugs prior to incorporation in a polymeric system to form a drug resin complex (aspect 171).

In aspects, the invention provides the method of aspect 170 or aspect 171, wherein the drug resin complex is further coated by release retarding polymer to form a controlled release drug resin complex (aspect 172).

In one aspect of the invention, the API in the pharmaceutical composition of any one or more of aspect A-aspect AD, aspect 1-aspect 154, and aspect 160 is mostly, generally, substantially, or entirely comprised of mexiletine or a pharmaceutically acceptable salt thereof (aspect 173).

In one aspect of the invention, the pharmaceutical composition of any one or more of aspect A-aspect AD, aspect 1-aspect 154, and aspect 160 does not comprise peptide(s) (aspect 174).

In one aspect of the invention, the pharmaceutical composition of any one or more of aspect A-aspect AD, aspect 1-aspect 154, and aspect 160 does not comprise lacosamide (aspect 175).

In one aspect of the invention, the pharmaceutical composition of any one or more of aspect A-aspect AD, aspect 1-aspect 154, and aspect 160 is only used for treating myotonic disorders (aspect 176).

In one aspect of the invention, the pharmaceutical composition of any one or more of aspect A-aspect AD, aspect 1-aspect 154, and aspect 160 is only used for treating disorders of skeletal muscles (aspect 177).

In one aspect of the invention, the pharmaceutical composition of any one or more of aspect A-aspect AD, aspect 1-aspect 154, and aspect 160 is not used for treating hyperexcitability disorders (aspect 178).

In one aspect of the invention, the pharmaceutical composition of any one or more of aspect A-aspect AD, aspect 1-aspect 154, and aspect 160 is not used for treating anxiety, and stress diseases (aspect 179).

In one aspect of the invention, the pharmaceutical composition of any one or more of aspect A-aspect AD, aspect 1-aspect 154, and aspect 160 is not used for treating disorders of the central nervous system or the peripheral nervous system (aspect 180).

In one aspect of the invention, the pharmaceutical composition of any one or more of aspect A-aspect AD, aspect 1-aspect 154, and aspect 160 is not co-administered with another medication (aspect 181).

In one aspect of the invention, the API(s)s of the pharmaceutical composition of any one or more of aspect A-aspect AD, aspect 1-aspect 154, and aspect 160 contain(s) no amide bonds (aspect 182).

In one aspect of the invention, the API(s)s of the pharmaceutical composition of any one or more of aspect A-aspect AD, aspect 1-aspect 154, and aspect 160 only contain(s) one chiral carbon atom (aspect 183).

In aspects, the invention provides the product of any one or more of aspect 155-aspect 159 having any one or more characteristics of aspect A-aspect AD, aspect 1-aspect 154, aspect 160, or aspect 173-aspect 183 (aspect 184).

In aspects, the invention provides the method of any one or more of aspect 161-aspect 172, comprising administering to the patient an effective amount of a composition or product (as applicable) according to any one or more of aspect 1-aspect 160, wherein the composition (or product, as applicable) further comprises any one or more of the characteristics described in any one or more of aspect 173-184 (aspect 185).

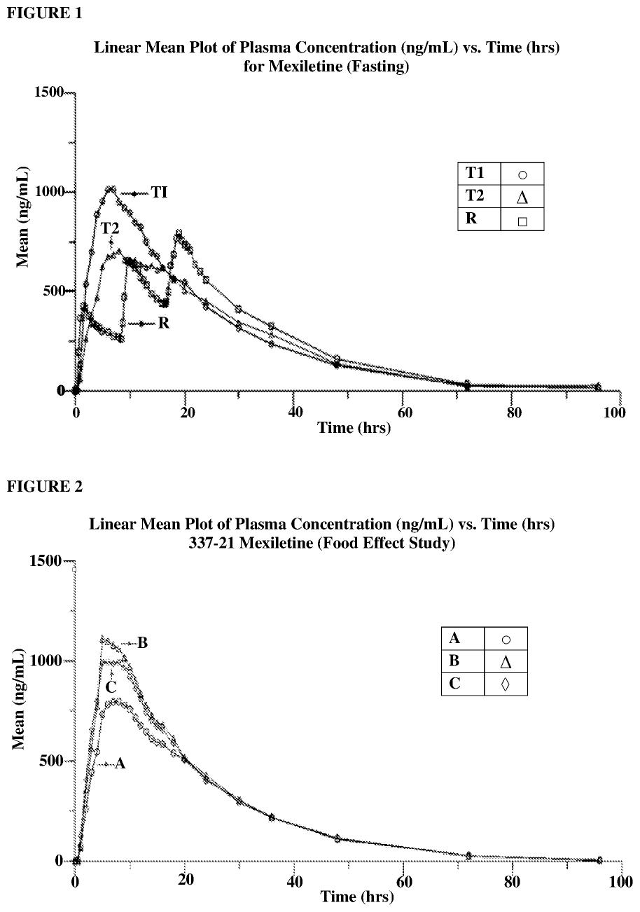

In an aspect, the invention provides a composition that optionally comprises any one or more of the characteristics, features, etc., of any one or more composition/formulation aspect described above, which exhibits characteristics that are significantly similar to the characteristics reflected in FIG. 1, FIG. 2, or both (or mostly or generally is significantly similar), or that are mostly, generally, or substantially similar to the compositions characterized in FIG. 1, FIG. 2, or both (e.g., in that most, i.e., ≥50% of the data obtained for such a composition is within +/−20%, 15%, 10%, or 5% of the data displayed in FIG. 1, FIG. 2, or both). In aspects, such a composition is a controlled release/delayed release composition comprising any one or more of the delayed release/controlled release compositions described in the following Detailed Description. In aspects, most of the release retardant agent of such a composition (or generally all or all) is an ion exchange resin release retardant agent or component.

### DETAILED DESCRIPTION OF THE INVENTION

To better illustrate the invention, descriptions of exemplary objects (e.g., devices, compositions, systems, etc.), methods, and individual elements thereof may be described in this Detailed Description of the Invention (“Detailed Description”).

Despite the inclusion of passages focused on specific elements/steps in this Detailed Description, uncontradicted, any aspect, facet, embodiment, or other description of a particular step(s) or element(s) can be applied to any other aspect.

Uncontradicted, any passage contained in this Detailed Description is to be interpreted as exemplary in nature and not intended to limit the scope of any possible aspect(s) of the invention provided by the disclosure.

### Composition/Composition Properties

In aspects, the present invention discloses controlled release pharmaceutical compositions and methods for their production. In aspects, compositions of the invention are formulated for oral administration. In aspects, compositions are in the form of or comprise a tablet, capsule, etc. In aspects, compositions that are adapted for oral administration and that comprise a therapeutically effective amount of mexiletine or a pharmaceutically acceptable salt thereof and a release retarding agent, wherein the release retarding agent effectively controls the release of the mexiletine or pharmaceutically salt thereof from the composition such that the release retarding agent causes the mexiletine or pharmaceutically acceptable salt thereof to be released from the composition after administration to human patients at a rate such that a single administration of the composition administered daily is effective to treat myotonia in a statistically significant number of myotonia patients over a treatment period of at least about a month, such as at least about three months (e.g., at least about 6, 12, 18, 24, 30, or 36 months, or longer). In aspects, the invention provides methods of using such compositions to treat conditions, such as myotonia, comprising administering an effective amount of such a composition to a patient, once a day, for a period, such as a period of at least 1 month, or 3, 6, 9, 12, 18, 24, 30, or 36 months, or longer, wherein the method does not comprise administering the composition to the patient more than once a day. In aspects, the methods and compositions are at least about as effective, if not detectably or significantly more effective, than comparator products, such as the on-market product known as Namuscla (e.g., TID 167 mg doses) which is described further herein and in the EMA 2018 document cited in the Background. Such compositions can, in some respects, be distinguishable from Namuscla in being effective throughout a treatment period with only once-daily administration. In aspects, the number of patients in which once-daily administration of the composition of the invention is effective is significantly greater than the number of patients treatable with once-daily administration of Namuscla.

In aspects, each dose of a composition as described in the preceding paragraph comprises about 100-about 600 mg of mexiletine hydrochloride, such as about 200-about 600 mg of mexiletine hydrochloride (e.g., ˜225-600 mg, ˜250-600 mg, ˜275-600 mg, ˜300-600 mg, ˜350-600 mg, ˜400-600 mg, or ˜450-600 mg, such as ˜100-550 mg, ˜100-500 mg, ˜100-450 mg, ˜100-400 mg, ˜100-300 mg, or ˜100-250 mg. In aspects, each dose of a composition comprises at least ˜200, 250, or ˜300 mg to no more than ˜600 mg, 550 mg, 500 mg, 450 mg, 400 mg, or ˜350 mg.

In aspects, the invention provides compositions according to either of the preceding paragraphs of this section, wherein the composition can be further characterized in exhibiting a median Tₘₐₓ of about 5 to about 8 hours (e.g., about 5.5-7.5 hours, such as about 6-7, about 5.75-6.75, about 5.75-6.25, or about 6 hours); a mean t_{1/2} about 10 hours to about 13.5 hours (e.g., about 10.5-13 hours, about 11-12.5 hours, about 10.5-12.5 hours, about 11-12 hours, about 10.5-12 hours, about 11 hours, about 12 hours, about 11-13 hours, or about 11-13.5 hours); or both such characteristics. The invention further provides compositions according to any of the preceding passages of this section, wherein the composition is further characterized in exhibiting at least one of the following pharmacokinetic profile characteristics after single dose administration: (a) a mean Cₘₐₓ about 700-1250 ng/mL; a mean AUC0-24 about 15500 ng*hr/mL to about 17250 ng*hr/mL; a mean AUC₀₋ₜ of about 22000-24250 ng*hr/mL; and a mean AUC_{0-∞} about 22500-26000 ng*hr/mL. In further aspects, the invention provides pharmaceutical compositions comprising an effective amount of mexiletine or a pharmaceutically acceptable salt thereof and an effective amount of a release retardant/retarding agent wherein the composition also (in combination with any other aspects of this section or disclosure) or alternatively (to any such aspects) exhibits at least one, such as at least two, such as 3 or more (e.g., 1-5, 2-5, 2-4, 2-3, 1-4, 1-3, 3, 4, 5, or all) of the following pharmacokinetic profile characteristics after single dose administration:
(g) median, Tₘₐₓ about 5 to about 8 hours;
(h) mean, Cₘₐₓ about 700-1250 ng/mL;
(i) mean, AUC0-24 about 12000 ng*hr/mL to about 17500 ng*hr/mL;
(j) mean, AUC₀₋ₜ of about 22000-24250 ng*hr/mL;
(k) mean, AUC_{0-∞} about 22500-26000 ng*hr/mL; and
(l) mean, t_{1/2} about 10 hours to about 13.5 hours.

In aspects, the compositions of the invention, such as the compositions described in any of the preceding parts of this section, further exhibit an increase in mean Cmax, mean AUC, or both, in a fed condition with respect to fasting condition.

In aspects, compositions provided herein comprise one or more pharmaceutically acceptable mexiletine compounds, possibly in addition to one or more other compounds useful in the treatment of a condition treatable by mexiletine, such as myotonia (e.g., in aspects, compositions provided herein comprise an effective amount of a mexiletine compound or suitable salt thereof and an effective amount of one or more other myotonia treating APIs). In aspects, such one or more pharmaceutically acceptable compounds is/are loaded onto one or more ion exchange resin(s). In some respects, compositions comprise a detectable or significant amount of uncoated drug-resin complexes. In some respects, compositions comprise a detectable or significant amount of coated drug-resin complexes. In aspects, compositions comprise a detectable or significant amount of both uncoated and coated drug-resin complexes.

### Mexiletine Compounds

In aspects, compositions provided herein comprise an effective amount of pharmaceutically acceptable compound(s) comprising mexiletine compound(s), such as, e.g., mexiletine. In aspects, such compounds can be provided in, e.g., any pharmaceutically acceptable form suitable for mammalian administration such as, e.g., a pharmaceutically acceptable salt(s), solvate(s), hydrate(s), enantiomer(s), derivative(s), polymorph(s), active metabolites, or prodrug(s) of such compound(s). In aspects, most, generally all, substantially all, essentially all, or all of the mexiletine compound of a composition/formulation or used in a method are mexiletine (e.g., as opposed to an analog or alternative form thereof).

Uncontradicted, any reference to mexiletine herein or mexiletine compound will be understood as implicitly simultaneously disclosing any and all suitable salts thereof (e.g., any reference to a composition comprising mexiletine herein should be understood as implicitly disclosing a corresponding composition comprising an equivalent amount of suitable mexiletine salt(s), such as mexiletine hydrochloride) and, where suitable, any disclosure of a salt of mexiletine or a mexiletine compound will be understood as providing implicit simultaneous disclosure of a corresponding method, composition, etc., comprising mexiletine or another suitable mexiletine compound which is not in any such salt form.

In aspects, compositions provided herein comprise an effective amount of mexiletine compound(s), e.g., mexiletine. Uncontradicted, any disclosure herein that uses the term mexiletine compound provides implicit support for a corresponding aspect wherein the mexiletine compound is mexiletine and vice versa (in each case including any suitable salts thereof). In aspects, a mexiletine compound can be any pharmaceutically acceptable mexiletine compound suitable for mammalian administration that has been identified or which is identifiable or which may be contemplated, such as, e.g., mexiletine or pharmaceutically acceptable salt(s), pharmaceutically acceptable solvate(s), pharmaceutically acceptable hydrate(s), pharmaceutically acceptable enantiomer(s), pharmaceutically acceptable geometrical isomer(s), pharmaceutically acceptable stereoisomer(s), pharmaceutically acceptable diastereomer(s), pharmaceutically acceptable N-oxide(s), pharmaceutically acceptable derivative(s), pharmaceutically acceptable polymorph(s), and pharmaceutically acceptable prodrug(s) thereof. In aspects, composition(s) can comprise a mixture of any one or more of mexiletine or pharmaceutically acceptable salt(s), solvate(s), hydrate(s), enantiomer(s), derivative(s), polymorph(s), or prodrug(s) of mexiletine.

In aspects, compositions herein can comprise a mexiletine compound which is, e.g., a salt of mexiletine, such as, e.g., mexiletine hydrochloride, or, e.g., mexiletine compound salts disclosed in, e.g., US20110028552, or, e.g., US20120196933. In aspects, compositions herein can comprise a mexiletine compound which is, e.g., mexiletine in solvated form(s) (such as hydrates), mexiletine in unsolvated form, or both. In aspects, the invention encompasses both such forms, such as those described by Andrews, et. al., in, “Derisking the Polymorph Landscape: The Complex Polymorphism of Mexiletine Hydrochloride,” Cryst. Growth Des. 2021, 21, 12, 7150-7167. In aspects, compositions herein can comprise a mexiletine compound which is, e.g., any pharmaceutically acceptable geometrical isomer(s), stereoisomer(s), enantiomer(s), diastereomer(s), or N-oxide(s) of mexiletine. Mexiletine or mexiletine compounds can exist in two or more stereoisomeric forms (e.g., diastereomers and enantiomers). In aspects, a mexiletine compound can extend to all known stereoisomeric forms of mexiletine and to mixtures thereof. Specific stereoisomeric forms of mexiletine provided herein can, in aspects, be separated from one another by methods known in the art, or, e.g., a given isomer can be obtained by, for example, stereospecific or asymmetric synthesis. In some respects, it may be particularly advantageous to use a particular isomeric form of mexiletine, such as, e.g., a form demonstrating detectably significantly higher levels of biological activity, bioavailability, or both. Exemplary forms of such mexiletine compounds are disclosed in, e.g., Tekewe, et. al., “Development and validation of HPLC method for the resolution of drug intermediates: DL-3-Phenyllactic acid, DL-O-acetyl-3-phenyllactice acid and (+/−)-mexiletine acetamide enantiomers,” Talanta 75 (2008) 239-245 and in numerous articles cited therein (see, e.g., references 1-15 cited therein.) In aspects, mexiletine compounds disclosed herein can be, e.g., any one or more such cited compounds or such compounds disclosed in the cited references. In aspects, compositions herein can comprise mexiletine compounds which are derivatives of mexiletine, such as, e.g., pyrroline derivatives of mexiletine, those disclosed in, e.g., Frederickson, et. al., “Fragment-Based Discovery of Mexiletine Derivatives as Orally Bioavailable Inhibitors of Urokinase-Type Plasminogen Activator,” J. Med. Chem., 2008, 51, 2, 183-186, or, e.g., those disclosed by Chau, et. al., in, “Synthesis of six mexiletine derivatives with isoindolines attached as potential antioxidants and their evaluation as cardioprotective agents,” MedChemComm, 2015, 6, 634-639. In aspects, mexiletine compound(s) herein can be a polymorph of mexiletine, such as, e.g., a polymorph of mexiletine hydrochloride as disclosed by Andrews, et. al., supra, or, e.g., in Kiss and Repasi, “Investigation of polymorphism of mexiletine hydrochloride by Fourier transform infrared and differential scanning calorimetric techniques,” Analyst, 1993, 118, 661-664. In aspects, a mexiletine compound can be a prodrug of mexiletine, such as those disclosed in, e.g., WO2012085586, US2011028552, or, e.g., in US20120196933. Herein, the use of the term “mexiletine compound(s)” should be interpreted as referencing any such mexiletine compound disclosed in this paragraph or other mexiletine compounds known in the art. In specific aspects, compositions herein comprise a mexiletine compound characterizable as a salt of mexiletine, e.g., mexiletine hydrochloride.

In certain specific aspects, the API(s) in the compositions is mostly comprised of, generally comprised of, substantially comprised of, essentially comprised of, or consist of mexiletine (a mexiletine compound) or a pharmaceutically acceptable salt thereof, e.g., mexiletine hydrochloride. In some respects, the API in the compositions consists of mexiletine or a pharmaceutically acceptable salt thereof, e.g., mexiletine hydrochloride. In aspects, the composition is substantially free, essentially free, or entirely free of any non-mexiletine API that does not contain amide bonds.

The mexiletine compound(s) or combination of mexiletine compound(s) and other API(s) of a composition can be referred to as an API component.

### Release Retardant Agents

In aspects, compositions can comprise release retardant/retarding agent(s). Compositions of the invention can comprise, e.g., a (one or more) release retardant/retarding agent(s) that impart any of the various release profile characteristics described above (e.g., a median, Tₘₐₓ about 5 to about 8 hours, the composition exhibits a mean t_{1/2} about 10 hours to about 13.5 hours, or both and/or the ability to provide a therapeutically effective amount of mexiletine to a patient by a single administration of a dose of the composition in a significant number of relevant patients, such as myotonia patients). The release retardant agent can be composed of any suitable number of release retarding compositions, present in any suitable amount. The release retardant agent is typically suitable in the amount used for achieving a controlled release effect, e.g., an effect that releases mexiletine compound(s) of a composition/formulation such that the agent renders the associated composition effective for once daily administration to a significant number, most, or generally all patients in a population and that is pharmaceutical suitable (e.g., not unacceptably toxic to at least most subjects if not generally all or substantially all subjects). Numerous types of release retardant materials are provided herein, and equivalents thereof known in the art. To exemplify such an embodiment, significant disclosure is provided herein regarding compositions comprising ion exchange resin components as a part of a release retardant component/element alone or with other release retardant agents/elements (e.g., one or more release retardant coating polymers). While such formulations are a unique aspect of the invention, such detailed disclosure is not intended to detract those of ordinary skill in the art from employing other release retardant agents to achieve compositions having the characteristics described herein, which the inventors conceive are suitable as therapeutic agents according to the various aspects of the invention provided throughout this disclosure. In aspects, the release retardant agent(s) of the formulation releases the mexiletine or pharmaceutically acceptable salt thereof (causes such API(s) to be released) in a controlled release manner when administered to a human subject, in aspects in a controlled release manner such that once-daily administration of the composition is effective for the treatment of myotonia (e.g., in a significant number of patients, most patients, etc.). In aspects, a composition comprises release retardant agent(s) in amount(s)/concentration(s) sufficient to impart release characteristics that are significantly similar to or DOS better than any one or more of the pharmacokinetic profiles described in this disclosure. In aspects, a composition comprises release retardant agent(s) in amount(s)/concentration(s) sufficient to impart release characteristics that are significantly similar to or DOS better than an ion exchange resin of the disclosure. Such characteristics can be combined with any other aspect of the extended/controlled release compositions described in the following section.

According to certain aspects, release retardant/retarding agent(s) are present as a coating. In aspects, release retardant/retarding agent(s) are present as a layered release retardant/retarding coating. In aspects, release two or more release retardant/retarding agent(s) are present as a layered coating wherein each layer of the release retardant/retarding coating is detectably or significantly different from one or more other layer(s) of the coating. In aspects, release retarding agent(s) can be one or more agent(s) described in the discussion of coating materials described herein or equivalents known in the art, e.g., delayed release polymers and other similar or equivalent types of agent(s) known in the art.

Release retardant/retarding agent(s) can be present in any suitable part of a composition and can comprise any suitable compound(s) in any suitable concentration(s). In some aspects, such agents are present as coatings. In aspects, such agents also or alternatively make up other parts of the composition (e.g., API-complexed moieties, groups, agents, etc.). Controlled release/delayed release agents and other release retarding agents are well studied and known in the art and any suitable agents known in the art can be used. In aspects, compositions comprise compound(s)/agent(s)/composition(s) described herein as coatings as possible release retarding agent(s), even if some or part of such compound(s)/agent(s)/composition(s) are not used solely as coating(s).

### Extended/Controlled Release Characteristics and Components

An important aspect of the characteristics of the compositions of the present invention is the controlled release of drug, e.g., mexiletine compound, contained therein. Such controlled release (e.g., extended-release), provides numerous benefits of the present invention over existing art in that it alleviates the requirement of more constant or continuous treatment administration to overcome the quick dissolution and quickly declining plasma concentrations of mexiletine administered in an immediate release form. Examples of compositions for achieving controlled/delayed release include sustained release coated microparticles or nanoparticles, pellets, microspheres, micelles, and similar components (e.g., in the form of oral suspensions) (e.g., polymeric micelle formulations, polymeric micelle formulations, etc.), hydrogels, sparingly soluble salt formulations, alginate-based in situ gelling systems or other polymeric in situ gelling systems, self-emulsifying drug delivery systems (SEDDS), microemulsions, liposomal formulations, and liquefied gas formulations. Disclosures in the art relating to such matters and related matters that may, in aspects, be applicable to or combinable with aspects of this disclosure are provided in, e.g., Oral Controlled Release Formulation Design and Drug Delivery: Theory to Practice (Hong Wen and Kinam Park Eds.) (2010) ISBN:9780470640487, DOI:10.1002/9780470640487); Haznar-Garbacz et al. AAPS PharmSciTech. 2011 December; 12(4):1183-5. doi: 10.1208/s12249-011-9689-8; Ebtessam A. Essa et al. Journal of Drug Delivery Science and Technology, Volume 61, 2021, 102211, ISSN 1773-2247, doi.org/10.1016/j.jddst.2020.102211; US20200315978A1; WO2011107855; U.S. Pat. No. 5,156,842A; CA2143070C; and WO2011107855A2. The agents that impart controlled release in such formulations can be characterized as release retarding agents or release retardants or controlled release agents/components herein (uncontradicted, such terms being exchangeable herein).

The pharmaceutical composition comprises one or more drugs, e.g., mexiletine compound(s), which are released slowly and in a controlled manner over the course of time and can be described as having a “controlled release.” According to certain embodiments, the term “controlled release” means the release of the drug, e.g., mexiletine compound, at a rate slower than immediate release. In aspects, controlled release means the release of drug, e.g., mexiletine compound after a single dose over the course of about 1 hour, ˜2 hours, ˜4 hours, ˜6 hours, ˜8 hours, ˜10 hours, ˜12 hours, ˜14 hours, ˜16 hours, ˜18 hours, ˜20 hours, ˜22 hours, or ˜24 hours or more, such as, e.g., ˜1 hour-˜24 hours, ˜2 hours-˜20 hours, ˜1 hour-˜16 hours, ˜1 hour-˜12 hours, or ˜1 hour-˜8 hours, e.g., ˜2 hours-˜24 hours, ˜6 hours-˜24 hours, ˜10 hours-˜24 hours, ˜14 hours-˜24 hours, ˜18 hours-˜24 hours, or ˜22 hours-˜24 hours, such as for example, ˜2 hours-˜22 hours, ˜6 hours-˜18 hours, or ˜10 hours-˜14 hours. In some respects, the mexiletine compound(s) of compositions provided herein can be released over a period of 24 hours or more after administration. In aspects, such characteristics are reflected in, e.g., a model, such as a dissolution model, e.g., dissolution in simulated gastric fluid or other media.

The long-acting release characteristic of the pharmaceutical composition(s) of the present invention can support a therapeutic administration schedule of such composition(s) wherein the composition(s) is administered no more than three times daily, for example, about once every ˜8 hours, once every ˜12 hours, once every ˜16 hours, once every ˜20 hours, or, e.g., once every ˜24 hours, e.g., once every ˜8-˜24 hours, ˜8 hours-˜20 hours, ˜8 hours-˜16 hours, ˜8 hours-˜12 hours, or once every ˜10 hours-˜24 hours, ˜14 hours-˜24 hours, ˜18 hours-˜24 hours, or ˜22 hours-˜24 hours. In aspects, compositions are administered no more than twice per day, such as, e.g., no more than once about every 12 to about every 24 hours, e.g., once every ˜16 hours-˜24 hours or once every ˜20 hours-˜24 hours, or about once every ˜12 hours-˜20 hours, ˜12 hours-˜16 hours, or, e.g., about once every ˜16 hours-˜20 hours. In aspects, compositions are administered no more than once per day, e.g., once every 24 hours.

In aspects, compositions provided by the invention are provided as a controlled release dosage form, wherein compositions comprise one or more drugs, e.g., one or more mexiletine compounds. In aspects, compositions provided by the invention are provided in extended-release form. In aspects, compositions herein are administered no more than once per about 12-hour, 14-hour, 16-hour, 18-hour, 20-hour, 22-hour, or 24-hour period. In aspects, compositions provided by the invention are administered once daily, e.g., once per 24-hour period. In aspects, compositions provided by the invention provide a mexiletine compound present in an extended-release form.

In particular aspects, the invention provides a controlled-release pharmaceutical composition comprising a mexiletine compound. In aspects, the invention provides a controlled release, a once-daily pharmaceutical composition comprising a mexiletine compound. In some respects, the mexiletine compound is a salt of mexiletine. In some respects, the mexiletine compound is complexed to an ion exchange resin.

In aspects, the invention provides a controlled release, once daily composition comprising a mexiletine compound which provides a detectably or significantly quicker, e.g., earlier, or faster, onset of therapeutic effect in the treatment of myotonia than current on-market product(s) (e.g., NaMuscla®). In aspects, such a therapeutic effect is observed within a time period that is at least about 1% faster than, ≥˜2%, ≥˜3%, ≥˜4%, ≥˜5%, ≥˜6%, ≥˜7%, ≥˜8%, ≥˜9%, ≥˜10%, ≥˜15%, ≥˜20%, ≥˜25%, ≥˜30%, ≥˜35%, ≥˜40%, ≥˜45%, or, e.g., ≥˜50% shorter than current on-market product(s) (e.g., NaMuscla®).

In aspects, the controlled release composition(s) provided herein provide clinically relevant efficacy in the treatment of myotonia for a detectably or significantly extended period of time compared to current on-market product(s) (e.g., NaMuscla®). In aspects, a single dose of a composition herein provides a clinically relevant efficacy in the treatment of myotonia for a period of time that is at least about 1% longer than, ≥˜2%, ≥˜3%, ≥˜4%, ≥˜5%, ≥˜6%, ≥˜7%, ≥˜8%, ≥˜9%, ≥˜10%, ≥˜15%, ≥˜20%, ≥˜25%, ≥˜30%, ≥˜35%, ≥˜40%, ≥˜45%, ≥˜50%, ≥˜55%, ≥˜60%, ≥˜65%, ≥˜70%, ≥˜75%, ≥˜80%, ≥˜85%, ≥˜90%, ≥˜95%, ≥˜100%, ≥˜150%, ≥˜200%, ≥˜250%, ≥˜300%, ≥˜350%, ≥˜400%, ≥˜450%, or, e.g., ≥˜500% longer than current on-market product(s) (e.g., NaMuscla®).

In aspects, compositions provided by the invention maintain a clinically relevant level of drug availability for a detectably or significantly extended period of time compared to current on-market product(s) (e.g., NaMuscla®). In aspects, compositions provided by the invention maintain a clinically relevant level of drug availability for a period of time which is at least about 1% longer than, ≥˜2%, ≥˜3%, ≥˜4%, ≥˜5%, ≥˜6%, ≥˜7%, ≥˜8%, ≥˜9%, ≥˜10%, ≥˜15%, ≥˜20%, ≥˜25%, ≥˜30%, ≥˜35%, ≥˜40%, ≥˜45%, ≥˜50%, ≥˜55%, ≥˜60%, ≥˜65%, ≥˜70%, ≥˜75%, ≥˜80%, ≥˜85%, ≥˜90%, ≥˜95%, ≥˜100%, ≥˜150%, ≥˜200%, ≥˜250%, ≥˜300%, ≥˜350%, ≥˜400%, ≥˜450%, or, e.g., ≥˜500% longer than current on-market product(s) (e.g., NaMuscla®).

Herein, a “therapeutically effective amount” typically means an amount of a compound or pharmaceutical composition that will elicit an intended (typically significant) biological or medical response of a tissue, system, animal, or human that is being sought by the researcher, medical doctor, or other clinician. In aspects, a therapeutically effective amount is demonstrated by at least one or at least two well-controlled and adequate clinical studies in human subjects/patients (e.g., as would be considered sufficient for pharmaceutical approval). Herein, “clinically relevant efficacy” typically means demonstrating a detectable or significant intended effect or biomedical or medical response (typically significant) in or on a targeted tissue, system, animal, or human sought by a researcher, medical doctor, or other clinician. Herein, a “clinically relevant level” of a drug is a level sufficient to yield a detectable or significant intended effect or biomedical or medical response (typically significant) in or on a targeted tissue, system, animal, or human.

### Ionic Exchange Resins

In aspects, compositions provided by the invention comprise a drug loaded onto an ion exchange resin, e.g., ion exchange resin particles. In aspects, ion exchange resin particles are loaded with pharmaceutical drugs prior to incorporation in a polymeric system to form a drug-resin complex. In some respects, the drug-resin complex is left uncoated. In some respects, the drug-resin complex is further coated with one or more release retarding polymer(s) to form a controlled-release drug-resin complex.

Ion exchange resins are water-insoluble polymers in the form of very small particles and beads. Though the properties of ion exchange resins may vary depending upon the intended application, in some respects, they generally consist of an insoluble porous polymer lattice or matrix with attached ionic functional groups. In aspects, a drug disclosed herein, e.g., a mexiletine compound, is bound to the ion exchange resin by an acid-base reaction. In certain aspects, compositions comprise a cation exchange resin. In certain aspects, compositions comprise an anion exchange resin. In aspects, compositions can comprise a combination of cation and anion exchange resins. In some respects, the ion exchange resin is selected based on the drug to be bound to the resin. In aspects, ion exchange resin(s) are selected based on the acidic or basic nature of the drug. In aspects, a basic drug can be bound to a cation exchange resin and an acid drug can be bound to an anion exchange resin. In aspects, compositions comprise a mexiletine compound, wherein the mexiletine compound is bound to, e.g., a cationic ion exchange resin.

In aspects, any pharmaceutically suitable, e.g., biologically safe (“biologically safe” meaning not causing detectable or significant negative or detrimental effect(s) on the health of a patient in receipt thereof, such as, e.g., a detectable or significant negative effect on the function of one or more tissues or systems of a patient) ion exchange resin can be used in the preparation of drug-ion exchange resin complex(s) of the composition, such as, e.g., the mexiletine-ion exchange resin complex(es) of the present invention, provided it is pharmaceutically acceptable and has a suitable particle size or bead diameter.

In certain aspects, compositions herein comprise ion exchange resin particles having a particle size of at least about 20 μm, such as, e.g., ≥˜20 μm, ≥˜22 μm, ≥˜24 μm, ≥˜26 μm, ≥˜28 μm, ≥˜30 μm, ≥˜32 μm, ≥˜34 μm, ≥˜36 μm, ≥˜38 μm, or, e.g., ≥˜40 m. In aspects, compositions comprise ion exchange resin particles having a particle size of 40 m or more, e.g., ≥˜42 μm, ≥˜44 μm, ≥˜46 μm, ≥˜48 μm, ≥˜50 μm, ≥˜52 μm, ≥˜54 μm, ≥˜56 μm, ≥˜58 μm, ≥˜60 μm, or, e.g., ≥˜70 μm, ≥˜80 μm, ≥˜90 μm, ≥˜100 μm, ≥˜120 μm, ≥˜140 μm, ≥˜160 μm, ≥˜180 μm, ≥˜200 μm, ≥˜220 μm, ≥˜240 μm, ≥˜260 μm, ≥˜280 μm, or ≥˜300 m.

All commercially available ion exchange resins are contemplated as being within the scope of the present invention. In aspects, ion exchange resins useful in the practice of the present invention include, but are not limited to, e.g., anionic resins such as DUOLITE® AP143/1083 (cholestyramine resin USP) and cationic resins such as AMBERLITE® IRP-64 (a porous copolymer of methacrylic acid crosslinked with divinylbenzene), AMBERLITE® IRP-69 (Sodium polystyrene sulfonate USP) and AMBERLITE® IRP-88. In certain aspects, compositions comprise AMBERLITE® IRP 69 resins. AMBERLITE® IRP 69 (sodium polystyrene sulfonate) is available commercially as a sodium salt. However, it is within the scope of the invention to convert the sodium salt to other salt forms, including, but not limited to, K and Li. Therefore, in certain aspects, AMBERLITE® IRP 69 resins are provided as a potassium salt, AMBERLITE® IRP 69 resins are provided as a potassium salt, lithium salt, or both.

### Drug-Resin Complex

In aspects, the drug-resin complex is formed by placing the ion-exchange resin, e.g., the ion-exchange resin selected based upon one or more characteristics of the drug to which it is to be complexed, in an aqueous solution of the chosen form of drug. In some aspects, the combination is mixed or otherwise agitated. In aspects, drug-resin complex(ex) formed are collected and washed. In aspects, complexes are washed with deionized or purified water. In aspects, washing ensures the removal of generally all, substantially all, or all unbound drug (e.g., drug not bound to an ion exchange resin particle). In aspects, the complexes are then dried. In certain aspects, the drug is a mexiletine compound, such as, e.g., a salt of mexiletine. In some respects, the ion exchange resin is a cationic exchange resin. In aspects, the cationic exchange resin is, e.g., AMBERLITE® IRP 69 resin(s) or a resin sharing at least generally the same, at least substantially the same, essentially the same, or the same characteristics as AMBERLITE® IRP 69 resin(s).

According to certain embodiments, the ion exchange resin is maximally loaded with drug, e.g., an ion exchange resin is loaded with a maximum amount of drug the resin can hold. In aspects, an ion exchange resin can be loaded with an amount which is below its maximum binding capacity, such as being loaded with an amount representing between about 10% and about 99%, e.g., ˜20%-˜99%, ˜30%-˜99%, ˜40%-˜99%, ˜50%-˜99%, ˜60%-˜99%, ˜70%-˜99%, or ˜80%-˜99%, or ˜90%-˜99%, e.g., ˜10%-˜90%, ˜10%-˜80%, ˜10%-˜70%, ˜10%-˜60%, ˜10%-˜50%, ˜10%-˜40%, ˜10%-˜30%, or, e.g., ˜10%-˜20% of its maximum drug binding capacity, as in, e.g., about 10%, ˜15%, ˜20%, ˜25%, ˜30%, ˜35%, ˜40%, ˜45%, ˜50%, ˜55%, ˜60%, ˜65%, ˜70%, ˜75%, ˜80%, ˜85%, ˜90%, or, e.g., ˜95%, ˜97.5%, or ˜99%, of its maximum drug binding capacity. In some aspects, such an amount is selected based on the desired dose of drug to be delivered. In aspects, such amounts disclosed here can be provided for drug-resin complexes wherein the drug is, e.g., a mexiletine compound and wherein the resin is cationic exchange resin, such as, e.g., AMBERLITE® IRP 69 resin(s) or resin(s) sharing at least generally the same, at least substantially the same, essentially the same, or the same characteristics as AMBERLITE® IRP 69 resin(s).

In aspects, compositions herein detectably or significantly reduce, or, e.g., at least generally, at least substantially, or essentially eliminate the bitter taste of mexiletine by means of complexation of a mexiletine compound with a cation exchange resin.

In aspects, the loading of drug, e.g., a mexiletine compound onto a resin, e.g., AMBERLITE® IRP 69 resin(s) or resin(s) sharing at least generally the same, at least substantially the same, essentially the same, or the same characteristics as AMBERLITE® IRP 69 resin(s), can be accomplished by well-known techniques. In aspects, batch-wise loading can be used, wherein a drug solution is mixed with resin in a suitable container for the time necessary to obtain maximal loading. In alternative aspects, a solution of the drug can be passed through a column of resin until loading is complete.

In aspects, drug-resin complexes further undergoing a coating process can be dried before coating.

### Drug-Resin Complex Coating

In aspects, pharmaceutical compositions comprise drug-resin complex(es), such as, e.g., mexiletine-resin complexes, wherein at least a portion of such complexes are coated with a release-retarding coating, e.g., a release-retarding polymeric coating. In aspects, pharmaceutical compositions comprising drug-resin complexes coated with a release retarding polymeric coating, such as, e.g., mexiletine compound-resin complexes coated with a release retarding polymeric coating, provide a controllable release of drug, e.g., a controlled release of a mexiletine compound, such as, e.g., a pharmaceutically acceptable salt of mexiletine, for a duration of at least about 10 hours, ≥˜12 hours, ≥˜14 hours, ≥˜16 hours, ≥˜18 hours, ≥˜20 hours, ≥˜22 hours, or ≥˜24 hours, such as, e.g., for a duration of at least about 24 hours, e.g., for a period of 24 hours or more.

In some aspects, compositions comprise a means for retarding release of the mexiletine compound(s). In aspects, such a means can be any means by which extended release, controlled release, or both, are accomplished in the art, such as, e.g., by encapsulation, coating, modifying the type of, chemistry related to, or degree of, binding of a drug to a resin, or any other known mechanisms of the art. In aspects, a means for retarding the release of mexiletine compound(s) in compositions herein is a coating means. In aspects, the coating means can be any coating means, such as, e.g., a single coating, a plurality of coatings, or, e.g., differing coating systems. In aspects, compositions herein comprise a means for retarding the release of mexiletine compounds characterizable as a coating means, e.g., comprising a polymeric coating system.

According to certain aspects, compositions provided by the invention comprise uncoated drug-resin complexes. In certain aspects, compositions provided by the invention comprise coated drug-resin complexes. In yet further aspects, compositions comprise both uncoated and coated drug-resin complexes. In aspects, a drug-resin complex coating can provide extended-release characteristics, e.g., extended drug-release characteristics, to the composition. In aspects, the amount of uncoated complex(es) and the amount of coated complex(es) in a composition can contribute to the drug release profile of the composition and, accordingly, the ratio of the uncoated to coated drug-resin complexes in the composition can be selected based upon a target drug release profile.

In certain embodiments, compositions herein comprise a first plurality of uncoated drug-resin particles and a second plurality of drug-resin particles being coated with a coating comprising at least one or more release retarding agents. In aspects, drug-resin complexes can comprise a single coating of release retarding agent(s). In aspects, drug-resin complexes can comprise two or more coatings of release retarding agent(s). In aspects, drug-resin complexes comprise two coatings of release retarding agent(s). In aspects herein, uncontradicted, reference to drug-resin particles being “coated,” or, e.g., references to the “coating” of drug-resin particles can be interpreted to mean drug-resin particles comprising a single coating or a plurality of coatings, each coating providing a detectable or significant amount of one or more release retarding agent(s).

In aspects, drug-loaded ion exchange resin particles, e.g., mexiletine compound-resin particle complexes are established, and the loaded resin particles are coated. In aspects, a coating material can comprise any of a large number of natural or synthetic film-formers used singly, in admixture with each other, and in admixture with plasticizers, pigments, and other substances to alter the characteristics of the coating. In aspects, a release retarding agent comprises a pharmaceutically acceptable polymer, hydrophilic or hydrophobic polymer such as ethyl cellulose (or ethylcellulose), methylcellulose, hydroxypropylmethylcellulose (or hydroxypropyl methylcellulose) (HPMC), hydroxyethylcellulose (or hydroxyethyl cellulose) (HEC), acrylic acid ester, e.g., Eudragit RL 100, Eudragit RLPO, Eudragit RL 30 D, Eudragit RSPO, Eudragit RS 30 D, Eudragit NE 30 D, Eudragit NE 40 D, cellulose acetate phthalate, HEC phthalate, HPMC phthalate or other cellulosic polymers, or mixtures of polymers.

In aspects, a release retarding agent of coated mexiletine compound ion exchange resin complex particles is in ranges from about 5% w/w to about 30% w/w of the total weight of coated complex composition, e.g., ˜10% w/w-˜30% w/w, ˜15% w/w-˜30%/w/w, ˜20% w/w-˜30%/w/w, or ˜25% w/w-˜30%/w/w, or, e.g., ˜5% w/w-˜25%/w/w, ˜5% w/w-˜20%/w/w, ˜5% w/w-˜15%/w/w, or ˜5% w/w-˜10% w/w of the total weight of the composition. In aspects, a release retarding agent comprises hydroxypropyl methyl cellulose, ethyl cellulose, or a combination thereof in a relative ratio range from about 30:70 to about 0:100, preferentially from about 25:75 to about 95:05 of the total weight of the release retarding polymer in the composition. This is also described elsewhere herein.

In certain aspects, a polymeric system, e.g., a release retarding polymeric system, will totally enclose mexiletine compound ion exchange resin complex particles when such particles are added thereto. In aspects, a polymeric system comprises one or more compounds described in this section. In aspects, a polymeric system comprises a plasticizer such as dibutylsebacate, vegetable oil, diethylsebacate, diethylphthalate, tricetin, or propylene glycol.

In aspects, once drug-resin complex(es) is/are formed, e.g., mexiletine compound-resin complexes are formed, complex(es) can be incorporated into a polymeric system. In aspects, such a polymeric system can be any polymeric system comprising one or more release retarding agents or compounds which detectably or significantly slow the release of drug-resin particles comprising the coating. In aspects, upon adding drug-resin complexes to the polymeric system (e.g., to the polymer coating), the polymer coating at least partially, such as, e.g., at least generally or at least substantially, or, e.g., completely, encloses each drug-resin (e.g., mexiletine-ion exchange resin) complex particle. In aspects, the polymeric coating at least partially, such as, e.g., at least generally, or at least substantially, or, e.g., completely, encloses at least 50%, ≥˜60%, ≥˜70%, ≥˜80%, ≥˜90%, ≥˜95%, ≥˜97.5%, ≥˜99%, or, e.g., all of the drug-resin (e.g., mexiletine-ion exchange resin) complex particles.

### Dosage Form

In aspects, compositions provided by the invention are provided in any suitable dosage form capable of delivering extended-release drug(s). In aspects, such a dosage form can be, e.g., swallowed, administered transdermally, transmucosally, or, e.g., other by methods known in the art. In some aspects, the delivery form can be an oral delivery form. In aspects, such an oral dosage form can be, e.g., solid dosage form, such as, e.g., a tablet, lozenge, or capsule comprising a solid composition, or, e.g., a liquid dosage form, such as, e.g., a liquid alone or provided within an enclosed delivery mechanism such as, e.g., a capsule. In aspects, a liquid oral dosage form can be, e.g., in the form of a solution. In aspects, a liquid oral dosage form can be, e.g., in the form of a suspension. In aspects, the liquid dosage form can be a liquid suspension provided within a capsule.

In aspects, compositions provided herein are provided in a liquid oral dosage form, such as, e.g., an aqueous solution, a non-aqueous solution, an emulsion, a suspension, or, e.g., solution(s) or suspension(s) reconstituted from granules or powders. In aspects, any dosage form described herein can comprise, e.g., one or more suitable solvents, preservatives, emulsifying agents, suspending agents, diluents, sweeteners, coloring (coloring) agents, flavoring (flavoring) agents, or excipients or carriers described herein.

According to certain aspects, coated complex(es) can be suspended in a substantially liquid carrier medium to form the liquid suspension composition. In one embodiment, the controlled release, e.g., extended-release, composition can readily be prepared as a liquid suspension for oral delivery at the time the product is used. In one embodiment, the invention provides a mexiletine controlled release formulation which is reconstitutable into an orally administered aqueous controlled release liquid suspension formulation.

In aspects, compositions provided herein can be provided as a dispersible tablet intended to be dispersed in water and administered as a draught with suspending controlled release mexiletine compound ion exchange complex.

In aspects, compositions provided herein are provided as dry powders or granules comprising one or more mexiletine compounds, such as, e.g., a salt of mexiletine. In aspects, such dry powders, granules, or, e.g., any other dosage form described herein, can be formulated into dosage forms of varying strengths by proportionally adjusting the amounts of the pharmaceutically acceptable excipients, as well as the active mexiletine compound.

In certain specific aspects, the invention provides an oral controlled release once daily composition comprising mexiletine compound(s), a pharmaceutically acceptable ion exchange resin, and a pharmaceutically acceptable carrier. In aspects, such a composition comprises mexiletine compound(s) complexed with a pharmaceutically acceptable ion exchange resin. In aspects, at least a portion of such mexiletine compound-resin complexes is coated by at least one coating comprising at least one release retarding polymer. In aspects, at least a portion of such mexiletine compound-resin complexes is coated by at least two coatings each comprising at least one release retarding polymer. In aspects, at least generally all, at least substantially all, or all of the mexiletine compound-resin complexes are coated by at least one coating comprising at least one release retarding polymer. In certain aspects, a composition wherein at least generally all, at least substantially all, or all of the mexiletine compound-resin complexes are coated by at least one coating comprising at least one release retarding polymer comprises at least a portion of the mexiletine compound-resin complexes comprising at least 2 coatings each comprising at least one release retarding polymer. In aspects, complexes of any such composition comprise complex(es) of a mexiletine compound and an ion exchange resin in the form of a powder, pellets, granules, or beads. In aspects, such a powder form, pelleted form, or granular form is supplied in the form of unit dose packs for example in sachet or in bottles, or as bulk multi-dose packs.

In aspects, compositions provided herein can be provided in unit doses. In aspects, compositions herein can be provided within packaging comprising multiple unit dosages. For example, in aspects, the invention provides for individually packaged liquid drug suspensions or the equivalent amount of drug as dispersible tablet compositions or in the form of dry powder or granules in preferred dosage forms. The dry powder or granular material can be dispensed in unit dose packs or multi-dose packs in the form of sachets or bottles (PET, HDPE & glass), blisters that can be reconstituted at the time of use into a suitable vehicle.

In aspects, coated drug-resin particles, e.g., mexiletine compound-resin particles, can be used in any suitable dosage form, e.g., a suspension, a chewable composition, an orally disintegrating composition, a capsule, a tablet, etc. In aspects, compositions of the present invention can be administered once per day in a single unit composition. In aspects, compositions of the present invention can be administered once per day in multi-unit composition. In aspects, a single unit or multi-unit composition provides a therapeutic benefit equivalent to or detectably or significantly greater than or better than (e.g., which improves upon) multiple doses of immediate release dosage form.

In one embodiment, multiple-unit pellet system(s) for a modified drug release (MUPS) composition can be prepared by loading mexiletine hydrochloride onto microcrystalline cellulose pellets by using povidone and talc. In aspects, pellets can be coated by hydroxymethyl propyl cellulose. In aspects, coated pellets can be coated a second time with ethyl cellulose and hydroxymethyl propyl cellulose.

In some aspects, the invention provides one or more advantages over prior art compositions and methods. For example, the mexiletine compound containing ion exchange complexes can be formulated into a number of different and easy-to-swallow dosage forms, including, but not limited to, liquid suspensions, granules, or dry powder for reconstitution.

Further, in aspects, coated or granulated mexiletine compound ion exchange resin complex(es) or mixtures of uncoated and coated mexiletine compound ion exchange resin complex can be filled in capsules of suitable size with or without lubricant or can be compressed into tablets with suitable excipients. In aspects, capsules provided herein can be swallowed by a patient directly or content(s) of capsules can be added to water or sprinkled on food immediately before ingestion.

### Amount/Dose

In one embodiment, a composition provided herein provides for administering an effective amount of a mexiletine compound. In aspects, an effective amount is an amount of between about 50 mg to about 1500 mg, such as, e.g., ˜50 mg-˜1400 mg, ˜50 mg-˜1300 mg, ˜50 mg-˜1200 mg, ˜50 mg-˜1100 mg, ˜50 mg-˜1000 mg, ˜50 mg-˜900 mg, ˜50 mg-˜800 mg, ˜50 mg-˜700 mg, or ˜50 mg-˜600 mg, e.g., ˜60 mg-˜1400 mg, ˜70 mg-˜1400 mg, ˜80 mg-˜1400 mg, ˜90 mg-˜1400 mg, ˜100 mg-˜1400 mg, ˜200 mg-˜1400 mg, ˜300 mg-˜1400 mg, ˜400 mg-˜1400 mg, ˜500 mg-˜1400 mg, or ˜600 mg-˜1400 mg, such as, e.g., ˜100 mg-˜1300, ˜100 mg-1200 mg, ˜100 mg-˜1000 mg, ˜100 mg-˜900 mg, ˜100 mg-˜800 mg, ˜100 mg-˜700 mg, or, e.g., ˜100 mg-˜600 mg.

In specific aspects, compositions herein provide for administering an effective amount of mexiletine compound for the treatment of myotonia in patients with dystrophic or non-dystrophic myotonic disorders, patients with myotonic dystrophy type 1, patients with myotonic dystrophy type 2, or any combination of any or all thereof, wherein the effective dosage can be, e.g., about 150 mg administered once per day, about 200 mg administered once per day, 300 mg administered once a day, 400 mg administered once per day, 500 mg administered once per day, or 600 mg administered once per day.

In aspects, compositions herein can be administered once per day (e.g., in a single unit or multiple unit compositions) in an amount that provides a therapeutic benefit equivalent to multiple doses of immediate-release dosage forms.

In aspects, the invention provides a controlled release once daily pharmaceutical composition of mexiletine compounds for administration once per 8-24-hour period (interval), e.g., once per 8-hour period, once per 10-hour period, once per 12-hour period, once per 14-hour period, once per 16-hour period, once per 18-hour period, once per 20-hour period, once per 22-hour period, or, e.g., once per 24-hour period.

### Dissolution Profile Characteristics

The release profile of the mexiletine ion exchange resin complex may be assessed via in vitro dissolution using techniques known to those skilled in the art.

In aspects, following administration of a single dose of the oral mexiletine compound controlled release, e.g., extended-release, in the form of a liquid suspension, a therapeutically effective amount of mexiletine is reached within about 60 minutes, such as, e.g., within about 50 minutes, ˜40 minutes, or within about 30 minutes of administration, such as, e.g., within about 25 minutes, ˜20 minutes, or within ˜15 minutes of administration and the formulation provides controlled release profile to at least about 12 hours, ˜14 hours, ˜16 hours, ˜18 hours, ˜20 hours, ˜22 hours, or, e.g., for ˜24 hours.

In aspects, pharmaceutical compositions comprising mexiletine compound ion exchange resin complex(es) of the present invention can exhibit an in vitro dissolution profile such that after 0-2 hours, from about 0% to about 70% by weight of mexiletine compound is released, such as, e.g., ˜0% w/w-60% w/w, ˜0% w/w-50% w/w, ˜0% w/w-40% w/w, ˜0% w/w-30% w/w, or ˜0% w/w-20% w/w of the mexiletine compound is released, such as, e.g., after 0-2 hours, from about 0% w/w to about 35% w/w of mexiletine compound is released, as measured using a USP type IV apparatus having a flow rate of about 8 ml/min along with 6 gm glass beads in 0.1N HCl media, 37° C.±0.5° C.

In aspects, pharmaceutical compositions comprising mexiletine compound ion exchange resin complex(es) of the present invention can exhibit an in vitro dissolution profile such that after 0-4 hours from about 10% w/w to about 85% w/w of mexiletine is released, e.g., ˜10% w/w-˜80% w/w, ˜10% w/w-˜70% w/w, ˜10% w/w-˜60% w/w, or such as, e.g., after 0-4 hours, from about 0% w/w to about 50% w/w, or, e.g., about 10% w/w to about 65% w/w or about 10% w/w to about 50% w/w of mexiletine is released, as measured using a USP type IV apparatus having a flow rate of about 8 ml/min along with 6 gm glass beads in 0.1N HCl media, 37° C.±0.5° C.

In aspects, pharmaceutical compositions comprising mexiletine compound ion exchange resin complex(es) of the present invention can exhibit an in vitro dissolution profile such that after 4-12 hours from about 30% w/w to about 100% w/w, such as, e.g., ˜30% w/w-˜90% w/w, ˜30% w/w-˜80% w/w, ˜30% w/w-˜70% w/w, or ˜30% w/w-˜60% w/w, or ˜40% w/w-˜100% w/w, ˜50% w/w-˜100% w/w, ˜60% w/w-˜100% w/w, ˜70% w/w-˜100% w/w, or ˜80% w/w-˜100% w/w, of the mexiletine compound is released, such as, e.g., after 4-12 hours, from about 30% w/w to about 80% w/w of mexiletine is released, as measured using a USP type IV apparatus having a flow rate of about 8 ml/min along with 6 gm glass beads in 0.1N HCl media, 37° C.±0.5° C.

In aspects, pharmaceutical compositions comprising mexiletine compound ion exchange resin complex(es) of the present invention can exhibit an in vitro dissolution profile such that after 0-2 hours, from about 0% w/w to about 30% w/w by weight of mexiletine is released, after 0-4 hours, from about 10% w/w to about 70% w/w by weight of mexiletine is released, after 4-12 hours, from about 30% w/w to about 100% w/w by weight of mexiletine is released, or any combination thereof when measured using a USP type IV apparatus having a flow rate of 8 ml/min along with 6 gm glass beads in pH 5.0 acetate buffer media, 37° C.±0.5° C.

In aspects, pharmaceutical compositions comprising mexiletine compound ion exchange resin complex(es) of the present invention can exhibit an in vitro dissolution profile such that after 0-1 hours, not less than about 50% w/w by weight of mexiletine is released, after 2 hours, not less than about 80% w/w by weight of mexiletine is released, after 4 hours, not less than about 90% w/w by weight of mexiletine is released, as measured using a USP type IV apparatus having a flow rate of 8 ml/min along with 6 gm glass beads in pH 6.8 phosphate buffer media, 37° C.±0.5° C.

In aspects, the rate of release of the mexiletine compound is controlled by the thickness of the release retarding agent, and therefore the coating thickness can, in aspects, be changed to vary the release rate to obtain the optimum dissolution and bioavailability profile.

In aspects, pharmaceutical compositions having coated mexiletine compound ion exchange resin complex(es) of the present invention may exhibit an in vitro dissolution profile such that after 0-4 hours, from about 10% w/w to about 65% w/w of mexiletine is released, after 4-12 hours, from about 30% w/w to about 90% w/w of mexiletine compound is released; when measured using a USP type IV apparatus having a flow rate 8 ml/min along with 6 gm glass beads in pH 6.8 phosphate buffer media, 37° C.±0.5° C.

In aspects, the pharmaceutical compositions of the present invention may exhibit an in vitro dissolution profile such that after 0-2 hours, from about 0% w/w to about 30% w/w of mexiletine compound is released, after 0-4 hours, from about 10% w/w to about 40% w/w of mexiletine compound is released, after 4-12 hours, from about 30% w/w to about 70% w/w of mexiletine is released, from 6-14 hours, more than about 60% w/w of mexiletine is released and, more than about 80% w/w by weight of mexiletine is released up to 24 hours when measured using a USP type IV apparatus having a flow rate of 8 ml/min along with 6 gm glass beads in 0.1N HCl media, 37° C.±0.5° C.

In aspects, the pharmaceutical compositions of the present invention may exhibit an in vitro dissolution profile such that after 0-2 hours, from about 0% w/w to ˜25% w/w of mexiletine compound is released, after 0-4 hours, from ˜10% w/w to ˜40% w/w of mexiletine compound is released, after 4-12 hours, from ˜30% w/w to about 65% w/w or more than 65% w/w of mexiletine is released, when measured using the USP type IV apparatus having flow rate 8 ml/min along with 6 gm glass beads in pH 5.0 acetate buffer media, 37° C.±0.5° C.

In another embodiment, the pharmaceutical compositions of the present invention may exhibit an in vitro dissolution profile such that after 0-2 hours, from about 0% w/w to about 35% w/w of mexiletine compound is released, after 0-4 hours, from about 10% w/w to about 50% w/w of mexiletine compound is released, after 4-12 hours, from about 30% w/w to about 75% w/w or more than 75% w/w of mexiletine is released, when measured using a USP type IV apparatus having a flow rate of 8 ml/min along with 6 gm glass beads in pH 6.8 phosphate buffer media, 37° C.±0.5° C.

In another embodiment, the pharmaceutical compositions of the present invention may exhibit an in vitro dissolution profile such that the composition releases not more than 50% w/w of mexiletine compound in an initial 1 hour in 500 mL 0.1N HCl and followed by 700 mL pH 4.5 acetate buffer changeover media the said composition releases not less than 65% w/w of mexiletine compound in 2 hours and further followed by 900 mL pH 6.8 phosphate buffer changeover media the said composition releases not less than 85% w/w of mexiletine compound in 4 hours when measured in United States Pharmacopoeia (USP) type II dissolution apparatus, rotated at 75 rpm at a temperature of 37°±0.5° C.

### Pharmacokinetic Characteristics

Compositions of the invention can be characterized by, i.a., the pharmacokinetic (PK) profile based on one or more PK characteristics/parameters exhibited by compositions of the invention when administered to subjects. In aspects, the release profile of the pharmaceutical composition of the present invention can be assessed in vivo (e.g., for relative bioavailability determinations), using plasma concentrations to assess maximum plasma concentration (Cₘₐₓ), area under the curve (AUC), and time to maximum plasma concentration (Tₘₐₓ). In aspects, any specific values provided in this section should be interpreted as representing a range less than about 20% of such a value to greater than ˜20% of such a value, including the extremes of such ranges, any value within such ranges to any order of magnitude, and, e.g., sub-ranges, thereof.

In aspects, the relative bioavailability can be measured as the concentration in the blood (serum or plasma) versus time area under the curve (AUC) determined for the test composition divided by the AUC in the blood provided by the reference composition. Preferably, this test/reference ratio is determined for each subject. In aspects, ratios are averaged over all subjects in a study.

In certain embodiments, the invention provides oral controlled release pharmaceutical compositions comprising mexiletine compounds and at least one release retarding agent, wherein a single dose once a day administration of the pharmaceutical compositions exhibits a median Tₘₐₓ about 6 or more hours, such as, e.g., ≥4, ≥5, ≥6, ≥7, or ≥8, such as, e.g., between about 4 to about 10, e.g., ˜4-˜9, ˜4-˜8, ˜4-˜7, or ˜4-˜6, e.g., ˜5-˜10, ˜6-˜10, ˜7-˜10, or ˜8-˜10, such as, for example, ˜5-˜7 hours.

In certain embodiments, the invention provides oral controlled release pharmaceutical compositions comprising mexiletine compounds and at least one release retarding agent, wherein the single dose once a day administration of the pharmaceutical compositions exhibits a mean Cₘₐₓ of about 763.2237 ng/mL or more, such as, e.g., ≥700 ng/mL, ≥800 ng/mL, ≥900 ng/mL, ≥1000 ng/mL, 1100 ng/mL, or ≥1200 ng/mL, such as, e.g., between about 700-1250 ng/mL, e.g., ˜700-˜1150 ng/mL, ˜700-˜1050 ng/mL, ˜700-˜950 ng/mL, or ˜700-˜850 ng/mL, e.g., ˜800-˜1250 ng/mL, ˜900-˜1250 ng/mL, ˜1000-˜1250 ng/mL, or ˜1100-˜1250 ng/mL, such as, for example, ˜800-˜1150 ng/mL.

In aspects, the invention provides oral controlled release pharmaceutical compositions comprising mexiletine compounds and at least one release retarding agent, wherein the single dose once a day administration of the pharmaceutical compositions exhibits a mean AUC₀₋₂₄ about 12826.7066 ng*hr/mL or more, such as, e.g., ≥15500 ng*hr/mL, ≥16500 ng*hr/mL, or ≥17250 ng*hr/mL, such as, e.g., between about 15500-17250 ng*hr/mL, e.g., ˜15500-˜17000 ng*hr/mL, ˜15500-˜16500 ng*hr/mL, or ˜15500-˜16000 ng*hr/mL, e.g., ˜15500-˜17250 ng*hr/mL, ˜16000-˜17250 ng*hr/mL, ˜16500-˜17250 ng*hr/mL, or ˜17000-˜17250 ng*hr/mL, such as, for example, ˜16000-˜16250 ng*hr/mL.

In aspects, the invention provides oral controlled release pharmaceutical compositions comprising mexiletine compounds and at least one release retarding agent, wherein the single dose once a day administration of the pharmaceutical compositions exhibits a mean AUC₀₋ₜ about 22301.7312 ng*hr/mL or more, such as e.g., ≥22000 ng*hr/mL, ≥22500 ng*hr/mL, ≥23000 ng*hr/mL, ≥23500 ng*hr/mL, or ≥24000 ng*hr/mL, such as, e.g., between about 22000-24250 ng*hr/mL, e.g., ˜22000-˜24000 ng*hr/mL, ˜22000-˜23500 ng*hr/mL, ˜22000-˜23000 ng*hr/mL, or ˜22000-˜22500 ng*hr/mL, e.g., ˜22500-˜24250 ng*hr/mL, ˜23000-˜24250 ng*hr/mL, ˜23500-˜24250 ng*hr/mL, or ˜24000-˜24250 ng*hr/mL, such as, for example, ˜22500-˜23250 ng*hr/mL.

In some embodiment, the invention provides oral controlled release pharmaceutical compositions comprising mexiletine compounds and at least one release retarding agent, wherein a single dose once a day administration of the pharmaceutical compositions exhibits a mean AUC_{0-∞} about 23345.7516 ng*hr/mL or more, such as e.g., ≥22500 ng*hr/mL, ≥23500 ng*hr/mL, ≥24500 ng*hr/mL, or ≥25500 ng*hr/mL, such as, e.g., between about 22500-26000 ng*hr/mL, e.g., ˜22500-˜25500 ng*hr/mL, ˜22500-˜25000 ng*hr/mL, ˜22500-˜24500 ng*hr/mL, ˜22500-˜24000 ng*hr/mL, or ˜22500-˜23500 ng*hr/mL, e.g., ˜23000-˜26000 ng*hr/mL, ˜23500-˜26000 ng*hr/mL, ˜24000-˜26000 ng*hr/mL, ˜24500-˜26000 ng*hr/mL, or ˜25000-˜26000 ng*hr/mL, such as e.g., ˜23000-˜25500 ng*hr/mL.

In aspects, the invention provides oral controlled release pharmaceutical compositions comprising mexiletine compounds and at least one release retarding agent, wherein the single dose once a day administration of the pharmaceutical compositions exhibits a mean t_{1/2} is about 12 hours or more, such as, e.g., ≥10 hours, ≥10.5 hours, ≥11 hours, ≥11.5 hours, ≥12 hours, ≥12.5 hours, or ≥13 hours, such as, e.g., between about 10 hours to about 13.5 hours, ˜10-˜13 hours, ˜10-˜12.5 hours, ˜10-˜12 hours, ˜10-˜11.5 hours, or ˜10-˜11 hours, e.g., ˜10.5-˜13.5 hours, ˜11-˜13.5 hours, ˜11.5-˜13.5 hours, ˜12-˜13.5 hours, ˜12.5-˜13.5 hours, or ˜13-˜13.5 hours, such as, for example, ˜11.5-˜13 hours.

In aspects, the invention provides oral controlled release pharmaceutical compositions comprising mexiletine compounds and at least one release retarding agent, wherein a single dose once a day administration of the pharmaceutical compositions exhibits mean Kel is about 0.05 or more, such as, e.g., ≥0.05, ≥0.06, ≥0.07, ≥0.08, or ≥0.09, such as, e.g., between about 0.05 to about 0.09, ˜0.05-˜0.08, ˜0.05-˜0.07, or ˜0.05-˜0.06, e.g., ˜0.06-˜0.09, ˜0.07-˜0.09, or ˜0.06-˜0.09, such as, for example, ˜0.06-˜0.08.

In aspects, the invention provides a controlled release once a daily pharmaceutical composition of mexiletine compound(s) complexed with a pharmaceutical acceptable ion exchange resin wherein the said composition exhibits steady-state concentration mexiletine compound in an amount of between about 0.4-2.5 g/mL, such as, e.g., between about 0.4 to about 2.0 g/mL, ˜0.4-˜1.5 g/mL, or ˜0.4-˜1.0 g/mL, e.g., ˜0.8-˜2.5 g/mL, ˜1.2-˜2.5 g/mL, ˜1.6-˜2.5 g/mL, or ˜2.0-˜2.5 g/mL, such as, for example, ˜1.5-˜2.0 g/mL.

In aspects, the invention provides an oral controlled release pharmaceutical composition comprising mexiletine compound(s) and at least one release retarding agent, wherein the pharmaceutical compositions exhibit a bioavailability relative to NaMuscla® based on the area under the plasma concentration curve (AUC) for the 24 hours after once a day administration in human subjects, which is greater than or equal to that of commercially available NaMuscla® capsules 167 mg administered thrice daily under fasting and fed conditions.

In aspects, the invention provides an oral controlled release pharmaceutical composition comprising mexiletine compound(s) and at least one release retarding agent, wherein the pharmaceutical compositions exhibit relative bioavailability of about 140%, such as, e.g., ˜130%, ˜135%, ˜145%, or ˜150%, for AUC₀₋₂₄.

In aspects, the invention provides oral controlled release pharmaceutical compositions comprising mexiletine compound(s) and at least one release retarding agent, wherein the pharmaceutical compositions exhibit relative bioavailability of about 130% for Cₘₐₓ.

In aspects, the invention provides an oral controlled release pharmaceutical composition comprising mexiletine compound(s) and at least one release retarding agent, wherein the pharmaceutical compositions exhibit relative bioavailability of about 105%, such as, e.g., ˜90%, ˜95%, ˜100%, ˜110%, or ˜115%, for AUC₀₋₂₄.

In aspects, the invention provides an oral controlled release pharmaceutical composition comprising mexiletine compound(s) and at least one release retarding agent, wherein the pharmaceutical compositions exhibit relative bioavailability of about 85%, such as, e.g., ˜75%, ˜80%, ˜90%, or ˜95%, for Cₘₐₓ.

In aspects, the invention provides an oral controlled release pharmaceutical composition comprising mexiletine compound(s) and at least one release retarding agent, wherein the pharmaceutical compositions exhibit an increase in Cₘₐₓ and AUC in the fed state compared to the fasted state.

In aspects, the invention provides an oral controlled release pharmaceutical compositions comprising mexiletine compound(s) and at least one release retarding agent, wherein the pharmaceutical compositions exhibit an increase in Cₘₐₓ of about 40%, such as, e.g., ˜30%, ˜35%, ˜45%, or ˜50%, and AUC of about 15%, such as, e.g., ˜5%, ˜10%, ˜20%, or ˜25%, of mexiletine hydrochloride when administer with food.

In aspects, mexiletine ion exchange resin complex(es) described herein can have one or more types of release profiles by coating the complexes with one or more release retarding agents and/or mixing particles with one or more different polymeric coatings to form a single composition and to maintain the plasma concentration of mexiletine compounds in a range of between about 0.4-2.5 μg/ml and more preferably between about 0.5-2 μg/ml, such as, e.g., between about 0.4 to about 2.0 g/mL, ˜0.4-˜1.5 g/mL, or ˜0.4-˜1.0 g/mL, e.g., ˜0.8-˜2.5 g/mL, ˜1.2-˜2.5 g/mL, ˜1.6-˜2.5 g/mL, or ˜2.0-˜2.5 g/mL, such as, for example, ˜1.5-˜2.0 g/mL.

### Stability Characteristics

In aspects, the invention provides pharmaceutical compositions that do not comprise any one impurity or a total amount/number of impurities that are deemed unsafe by one or more recognized regulatory bodies. In aspects, the invention provides pharmaceutical compositions that do not comprise any one degradation product or a total amount of degradation products that are deemed unsafe by one or more recognized regulatory bodies. (As an example, e.g., KOE-5692 (i.e., 2,3-Dihydro-3,9-dimethyl-1,4-benzoxazepine) is a cyclic imine impurity (degradation product) of mexiletine drug product).

In certain embodiments, the invention provides a controlled-release liquid suspension having improved delivery characteristics. In aspects, compositions of the invention remain stable over long periods of time when formulated in multiple-dose configurations. In aspects, compositions of the present invention are stable under ambient, 30° C./75% RH or 25° C./60% RH or 40°/75% RH stability conditions for a period of at least about 1, ≥˜2, ≥˜3, ≥˜4, ≥˜5, or, e.g., after at least about 6 months. In aspects, compositions provided herein are at least substantially free, essentially free, or are free of any significant amount, concentration, or number of impurities after at least about 1, ≥˜2, ≥˜3, ≥˜4, ≥˜5, or, e.g., after at least about 6 months. In aspects, compositions provided herein are at least substantially free, essentially free, or are free of any significant amount of impurities after at least about 1, ≥˜2, ≥˜3, ≥˜4, ≥˜5, or, e.g., after at least about 6 months as measured by an HPLC method, such as, e.g., the HPLC method described briefly here.

In aspects, an analytical method using HPLC is used to detect one or more impurities in compositions provided herein. In aspects, Inertsil ODS 3 v, 250×4.6 mm 5μ column is used and operated at a column temperature of 30° C. and UV wavelength of 262 nm. Mobile Phase A is, e.g., a buffer, while mobile phase B is, e.g., a mixture of acetonitrile and methanol. The sample diluent is, e.g., a mixture of sodium acetate buffer with 1.5% calcium chloride solution and Methanol with an injection volume of 10 μL. A mobile phase gradient (e.g., a mobile phase gradient program for Blank, placebo, and Sample) is shown below in Table 1.

TABLE 1
Gradient Program for Blank, Placebo, and Sample.

| Time in minutes | % Mobile Phase A | % Mobile Phase B |
|---|---|---|
| 0 | 85 | 15 |
| 30 | 75 | 25 |
| 45 | 55 | 45 |
| 60 | 50 | 50 |
| 65 | 35 | 65 |
| 70 | 35 | 65 |
| 75 | 85 | 15 |
| 80 | 85 | 15 |

In some aspects, compositions provided by the invention possess high stability as shown by the data disclosed herein. In aspects, no significant mexiletine compound degradation is observed in stability studies of compositions provided by the invention when stored under recommended storage conditions. In aspects, no significant mexiletine compound degradation is observed in stability studies of compositions provided by the invention when stored under recommended storage conditions for commercially relevant period(s) of time.

### Additional Excipients

In aspects, pharmaceutical compositions comprising a drug-resin complex can further comprise one or more pharmaceutically and physiologically acceptable excipients. In aspects, the pharmaceutically acceptable excipients can be, e.g., any number of recognized excipients in the art, such as those characterizable as fillers, binders, bulking agents, diluents, disintegrants, glidants, coloring agents, anti-adherents, dispersing agents, lubricants, coatings, preservatives, antioxidants, flavoring agents, sweetening agents, sorbents, solvents, co-solvents, buffering agents, chelating agents, viscosity imparting agents (viscosity modulating agents), surface active agents, humectants, emulsifiers, etc. In aspects, such an excipient can be any pharmaceutically acceptable excipient that aids in the manufacturing of the dosage form, aids in the performance of the dosage form, or both, but does not impart significant pharmaceutical activity (e.g., is not an active pharmaceutical ingredient (API)). In aspects, pharmaceutical compositions comprising a drug-resin complex can comprise one or more pharmaceutically acceptable and physiologically suitable carriers. In aspects, one or more components of a composition can provide a plurality of detectable or significant activities, such as, e.g., a single component can provide, e.g., detectable or significant suspension activity in addition to one or more of detectable or significant dispersion activity, viscosity modulating activity, or, e.g., emulsification activity.

In some aspects, the formulation is established according to the targeted route of administration. Any of the well-known accepted formulation techniques, composition carriers, and composition excipients may be used as is/are suitable and as is/are understood in the art. Exemplary components suitable for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N. Y., 1980; and Pharmaceutical Dosage Forms and Drug Delivery Systems, Seventh Ed. (Lippincott Williams & Wilkins 1999), herein incorporated by reference in their entirety.

In some embodiments, compositions provided herein comprise one or more preservatives in an amount suitable for detectably or significantly inhibiting microbial activity or in an amount sufficient for inhibiting microbial activity (e.g., in suitable anti-microbial concentrations). Suitable preservatives include mercury-containing substances such as merfen, thiomersal, etc.; stabilized chlorine dioxide; and quaternary ammonium compounds such as benzalkonium chloride, cetyltrimethylammonium bromide, and cetylpyridinium chloride, etc., and, e.g., other pharmaceutically acceptable and physiologically suitable preservatives known in the art.

In certain aspects, compositions herein comprise one or more dispersing agents, viscosity-modulating agents, or emulsifying agents. Dispersing agents, and/or viscosity modulating agents or emulsifying agents can include, e.g., materials that control the diffusion and homogeneity of a drug through liquid media or a granulation method or blend method. In some embodiments, these agents can facilitate the effectiveness of a coating or eroding matrix. In aspects, exemplary diffusion facilitators/dispersing agents include, e.g., hydrophilic polymers, electrolytes, Tween® 60 or 80 (e.g., polysorbate 60 or polysorbate 80), PEG, polyvinylpyrrolidone (PVP; commercially known as Plasdone®), and carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide), poloxamines (e.g., Tetronic 908®, also known as Poloxamine 908®, a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, polyvinylpyrrolidone K30, polyvinylpyrrolidone/vinyl acetate copolymer (S-630), polyethylene glycol, e.g., a polyethylene glycol having a molecular weight of about 300 to about 6000, or about 3350 to about 4000, or about 7000 to about 5400 g/mol), alginates such as sodium alginate, gums (such as, e.g., gum tragacanth and gum acacia, guar gum, xanthans, including xanthan gum), sugars, polyethoxylated sorbitan monolaurate, polyethoxylated sorbitan monolaurate, povidone, carbomers, polyvinyl alcohol (PVA), chitosans, and combinations thereof. Plasticizers such as cellulose or triethyl cellulose can also be used as dispersing agents. Dispersing agents particularly useful in, e.g., liposomal dispersions and self-emulsifying dispersions are dimyristoyl phosphatidyl choline, natural phosphatidyl choline from eggs, natural phosphatidyl glycerol from eggs, cholesterol, and isopropyl myristate.

In aspects, compositions herein can comprise, e.g., one or more flavoring agents or sweeteners. In aspects, flavoring agents and/or sweeteners useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and combinations or mixtures thereof.

In aspects, compositions provided by the invention can comprise one or more suspension (suspending) agents. In aspects, suspending agents (suspension agents) can include, e.g., compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxy ethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone, etc.

In aspects, compositions provided by the invention can comprise one or more lubricants or glidants. In aspects, a lubricant or glidant is a compound that detectably or significantly prevents, reduces, or inhibits adhesion or friction of materials. Exemplary lubricants can include, e.g., stearic acid, calcium hydroxide, talc, sodium stearyl fumarate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, etc.

In aspects, pharmaceutical compositions provided by the invention can comprise a carrier. In some aspects, the carrier can be a liquid carrier. In some aspects, the carrier can be an oil-based carrier. In some respects, the carrier can be a solid carrier. In aspects, a carrier in which drug-resin complexes of the present invention are suspended optionally comprises one or more pharmaceutically acceptable excipients well known in the art. In aspects, exemplary ingredients can include, e.g., one or more sweeteners, preservatives such as methyl paraben, propylparaben, or combinations thereof; and one or more emulsifying agents such as, e.g., polysorbate 80 or polysorbate 60 (e.g., Tween 80 or Tween 60). In aspects, a carrier can comprise one or more thickening agents such as guar gum or xanthan gum; one or more humectants such as propylene glycol or glycerin; one or more flavoring agents; one or more coloring agents; one or more suspending agents; one or more de-flocculating agents; one or more viscosity enhancers; one or more lubricants; one or more opacifiers; or combinations of any or all thereof.

### Relative Amounts and Relationship Between Components

In aspects, the amount of drug, e.g., mexiletine compound, loaded onto a resin can range from about 10% w/w to about 400% w/w, such as, e.g., ˜10% w/w-˜350% w/w, ˜10%-10% w/w-˜100% w/w, ˜10% w/w-˜50% w/w, such as, e.g., ˜50% w/w-˜400% w/w, ˜100% w/w-˜400% w/w, ˜150% w/w-˜400% w/w, ˜200% w/w-˜400% w/w, ˜250% w/w-˜400% w/w, ˜300% w/w-˜400% w/w, or ˜350% w/w-˜400% w/w, such as, e.g., ˜20% w/w-˜350% w/w, ˜30% w/w-˜300% w/w, ˜40% w/w-˜250% w/w, or, e.g., ˜50% w/w-˜200% w/w, or, e.g., ˜50% w/w-˜100% w/w by weight of the loaded mexiletine resin particles.

In aspects, the ion exchange resins useful in the practice of the present invention comprises from about 10% w/w to about 95% w/w by weight of the mexiletine ion exchange resin complex of the present invention, such as, e.g., ˜10% w/w-˜90% w/w, ˜10% w/w-˜80% w/w, ˜10% w/w-˜70% w/w, ˜10% w/w-˜60% w/w, ˜10% w/w-˜50% w/w, ˜10% w/w-˜40% w/w, ˜10% w/w-˜30% w/w, ˜10% w/w-˜20% w/w, or, e.g., ˜20% w/w-˜95% w/w, ˜30% w/w-˜95% w/w, ˜40% w/w-˜95% w/w, ˜50% w/w-˜95% w/w, ˜60% w/w-˜95% w/w, ˜70% w/w-˜95% w/w, ˜80% w/w-˜95% w/w or, e.g., ˜90% w/w-˜95% w/w.

In aspects, the drug loading of drug onto the resin prior to coating is between about 20% w/w and about 100% w/w by weight, such as, e.g., ˜30% w/w-˜100% w/w, ˜40% w/w-˜100% w/w, ˜50% w/w-˜100% w/w, ˜60% w/w-˜100% w/w, ˜70% w/w-˜10% w/w, ˜80% w/w-˜100% w/w, ˜90% w/w-˜100% w/w, or, e.g., about 100% w/w. As is described elsewhere herein, in aspects, one of skill in the art can selectively increase or decrease the amount of drug loaded on a resin particle to modify a drug release profile according to a target preference. In aspects, selective modification of the amount of drug loaded on a resin particle results in achieving a desired in vivo plasma concentration profile.

In aspects, compositions comprise a ratio of drug, e.g., mexiletine compound, e.g., salt of mexiletine, to ion exchange resin which ranges between about 1:0.25 to about 1:10, such as, e.g., ˜1:0.25-˜1:9, ˜1:0.25-˜1:8, ˜1:0.25-˜1:7, ˜1:0.25-˜1:6, ˜1:0.25-˜1:5, ˜1:0.25-˜1:4, ˜1:0.25-˜1:3, ˜1:0.25-˜1:2, or ˜1:0.25-˜1:1, e.g., ˜1:0.5-˜1:10, ˜1:0.75-˜1:10, ˜1:1-˜1:10, ˜1:2-˜1:10, ˜1:3-˜1:10, ˜1:4-˜1:10, ˜1:5-˜1:10, ˜1:6-˜1:10, ˜1:7-˜1:10, ˜1:8-˜1:10, or ˜1:9-˜1:10, such as, e.g., between about, e.g., 1:0.25 and about 1:5, such as, e.g., ˜1:0.5-˜1:4, ˜1:0.75:˜1:3, or, e.g., ˜1:1.

In aspects, the release retarding agent of the coated mexiletine ion exchange resin complex particles is present in an amount ranging from about 5% w/w to about 30% w/w of the total weight of a composition comprising coated complex(es), such as, e.g., in an amount of ˜5% w/w-˜25% w/w, ˜5% w/w-˜20% w/w, ˜5% w/w-˜15% w/w, or ˜5% w/w-˜10% w/w, such as, e.g., ˜10% w/w-˜30% w/w, ˜15% w/w-˜30% w/w, ˜20% w/w-˜30% w/w, or, e.g., ˜25% w/w-˜30% w/w, such as, e.g., ˜10% w/w-˜25% w/w of the total weight of composition comprising coated complex.

In aspects, compositions comprise one or more release retarding agents such as, e.g., hydroxypropyl methylcellulose, ethyl cellulose, or a combination thereof, wherein the ratio of the hydroxypropyl methylcellulose to ethyl cellulose is between about 30:70 (1:2.3) and about 0:100, such as, e.g., 25:75 (1:3) to about 95:05 (1:0.05).

In aspects, the amount of potassium polystyrene sulfonate (Amberlite IRP 69) is present in ranges from about 20% w/w to about 85% w/w, e.g., ˜40% w/w-˜85% w/w, ˜50% w/w-˜85% w/w, ˜60% w/w-˜85% w/w, ˜70% w/w-˜85% w/w, or ˜30% w/w-˜80% w/w, ˜40% w/w-˜80% w/w, ˜50% w/w-˜80% w/w, ˜60% w/w-˜80% w/w, or ˜70% w/w-˜80% w/w, as in, for example, ˜35% w/w-˜80% w/w, ˜40% w/w-˜70% w/w, ˜45% w/w-˜60% w/w, ˜45% w/w-˜55% w/w, or, e.g., about 50% w/w of the total composition.

In aspects, the amount of a release retarding first coat agent, e.g., coat 1 agent, e.g., hydroxy propyl methylcellulose (e.g., 3 mPa to 4000 mPa hydroxy propyl methylcellulose), is present in an amount ranging from about 1.7% w/w to about 1.9% w/w, such as, e.g., ˜1.75% w/w-˜1.9% w/w, ˜1.8% w/w-˜1.9% w/w, or ˜1.85% w/w-˜1.9% w/w, e.g., ˜1.7% w/w-˜1.85% w/w, ˜1.7% w/w-˜1.8% w/w, ˜1.7% w/w-˜1.75% w/w, e.g., ˜1.8% w/w of the total composition.

In aspects, the amount of a release retarding second coat agent, e.g., coat 2 agent, e.g., comprises hydroxy propyl methyl cellulose (e.g., 3 mPa to 4000 mPa hydroxy propyl methylcellulose), present in ranges from about 0.4% w/w to about 2.6% w/w, ethyl cellulose (e.g., 3 mPa to 330 mPa ethyl cellulose), present in ranges from about 3.3% w/w to about 7.8% w/w, and triethyl citrate, present in ranges from about 0.2% w/w to about 0.6% w/w. In aspects, such a second coat comprises hydroxy propyl methyl cellulose is an amount of about, e.g., 0.4% w/w-2.6% w/w, such as, e.g., ˜0.4% w/w-˜2.5% w/w, ˜0.4% w/w-˜2% w/w, ˜0.4% w/w-˜1.5% w/w, ˜0.4% w/w-˜1% w/w, e.g., ˜0.5% w/w-˜2.6% w/w, ˜1% w/w-˜2.6% w/w, ˜1.5% w/w-˜2.6% w/w, or ˜2% w/w-˜2.6% w/w e.g., ˜1% w/w-˜2% w/w of the total composition. In aspects, such a second coat comprises ethyl cellulose is an amount of about 3.3% w/w to about 7.8% w/w, e.g., ˜3.3% w/w-˜7.5% w/w, ˜3.3% w/w-˜7% w/w, ˜3.3% w/w-˜6.5% w/w, ˜3.3% w/w-˜6% w/w, ˜3.3% w/w-˜5.5% w/w, ˜3.3% w/w-˜5% w/w, ˜3.3% w/w-˜4.5% w/w, ˜3.3% w/w-˜4% w/w, or ˜3.3% w/w-˜3.5% w/w, e.g., ˜3.5% w/w-˜7.8% w/w, ˜4% w/w-˜7.8% w/w, ˜4.5% w/w-˜7.8% w/w, ˜5% w/w-˜7.8% w/w, ˜5.5% w/w-˜7.8% w/w, ˜6% w/w-˜7.8% w/w, ˜6.5% w/w-˜7.8% w/w, ˜7% w/w-˜7.8% w/w, or ˜7.5% w/w-˜7.8% w/w, e.g., ˜4% w/w-˜7% w/w of the total composition. In aspects, such a second coat comprises triethyl citrate in an amount of about 0.2% w/w-about 0.6% w/w, e.g., ˜0.2% w/w-˜0.55% w/w, ˜0.2% w/w-˜0.5% w/w, ˜0.2% w/w-˜0.45% w/w, ˜0.2% w/w-˜0.4% w/w, ˜0.2% w/w-˜0.35% w/w, ˜0.2% w/w-˜0.3% w/w, ˜0.2% w/w-˜0.25% w/w, e.g., ˜0.25% w/w-˜0.6% w/w, ˜0.3% w/w-˜0.6% w/w, ˜0.35% w/w-˜0.6% w/w, ˜0.4% w/w-˜0.6% w/w, ˜0.45% w/w-˜0.6% w/w, ˜0.5% w/w-˜0.6% w/w, or ˜0.55% w/w-˜0.6% w/w, e.g., ˜0.3% w/w-˜0.5% w/w of the total composition.

In aspects, the dispersible tablet composition comprises extended release coated mexiletine compound, e.g., a mexiletine hydrochloride, e.g., mexiletine HCl resinate, present in an amount of between about 60% w/w to about 70% w/w, such as, e.g., ˜60% w/w-˜68% w/w, ˜60% w/w-˜66% w/w, ˜60% w/w-˜64% w/w, ˜60% w/w-˜62% w/w, e.g., ˜62% w/w-˜70% w/w, ˜64% w/w-˜70% w/w, ˜66% w/w-˜70% w/w, or ˜68% w/w-˜70% w/w, such as, e.g., ˜62% w/w-˜68% w/w, or, e.g., ˜62% w/w-˜65% w/w of the total composition.

In aspects, the composition comprises crospovidone (or cross povidone) in an amount representing between about 3% w/w and about 6% w/w of the composition, e.g., ˜3% w/w-˜5.5% w/w, ˜3% w/w-˜5% w/w, ˜3% w/w-˜4.5% w/w, ˜3% w/w-˜4% w/w, or ˜3% w/w-˜3.5% w/w, e.g., ˜3.5% w/w-˜6% w/w, ˜4% w/w-˜6% w/w, ˜4.5% w/w-˜6% w/w, ˜5% w/w-˜6% w/w, or ˜5.5% w/w-˜6% w/w, such as, e.g., ˜4% w/w-˜5% w/w of the composition.

In aspects, compositions comprise microcrystalline cellulose present in an amount of between about 10% w/w and about 15% w/w, e.g., ˜10% w/w-˜14% w/w, ˜10% w/w-˜13% w/w, ˜10% w/w-˜12% w/w, ˜10% w/w-˜11% w/w, or, e.g., ˜11% w/w-˜15% w/w, ˜12% w/w-˜15% w/w, ˜13% w/w-˜15% w/w, ˜14% w/w-˜15% w/w, such as, e.g., ˜12% w/w-˜15% w/w, or ˜13% w/w-˜15% w/w.

In aspects, compositions comprise col. silicon dioxide in an amount of between about 0.5% w/w and about 2% w/w, e.g., ˜0.5% w/w-˜1.5% w/w, or ˜0.5% w/w-1% w/w, e.g., ˜1% w/w-˜2% w/w, or ˜1.5% w/w-˜2% w/w, e.g., ˜0.8% w/w-˜1.2% w/w of the total composition.

In aspects, compositions comprise sucralose in an amount of between about 1% w/w and about 5% w/w, such as, e.g., ˜1% w/w-˜4% w/w, ˜1% w/w-˜3% w/w, or ˜1% w/w-˜2% w/w, e.g., ˜2% w/w-˜5% w/w, ˜3% w/w-˜5% w/w, or ˜4% w/w-˜5% w/w, such as, e.g., about 2% w/w to 3% w/w of the total composition.

In aspects, compositions comprise a flavoring, e.g., a strawberry flavor/flavoring (flavor/flavoring), in an amount of between about 1% w/w and about 5% w/w, such as, e.g., ˜1% w/w-˜4% w/w, ˜1% w/w-˜3% w/w, or ˜1% w/w-˜2% w/w, e.g., ˜2% w/w-˜5% w/w, ˜3% w/w-˜5% w/w, or ˜4% w/w-˜5% w/w, such as, e.g., about 2% w/w to 3% w/w of the total composition.

In aspects, compositions comprise microcrystalline cellulose in an amount of between about 10% w/w and about 15% w/w, e.g., ˜10% w/w-˜14% w/w, ˜10% w/w-˜13% w/w, ˜10% w/w-˜12% w/w, ˜10% w/w-˜11% w/w, or, e.g., ˜11% w/w-˜15% w/w, ˜12% w/w-˜15% w/w, ˜13% w/w-˜15% w/w, ˜14% w/w-˜15% w/w, such as, e.g., ˜12% w/w-˜15% w/w, or ˜13% w/w-˜15% w/w, or, e.g., ˜12% w/w-˜13% w/w.

In aspects, compositions comprise magnesium stearate in an amount of between about 0.2% w/w and about 5% w/w, such as, e.g., ˜0.2% w/w-˜4% w/w, ˜0.2% w/w-˜3% w/w, or ˜0.2% w/w-˜2% w/w, e.g., ˜0.4% w/w-˜5% w/w, ˜0.6% w/w-˜5% w/w, or ˜0.8% w/w-˜5% w/w, such as, e.g., about 0.4% w/w to 3% w/w, 0.6% w/w to 2% w/w, or, e.g., 0.8% w/w to 1.2% w/w of the total composition.

### Exclusions/Excluded Elements

In one embodiment, the present invention provides a controlled release pharmaceutical composition administered no more than once per 12-hour, 14-hour, 16-hour, 18-hour, 20-hour, 22-hour, or 24-hour period comprising a drug, e.g., a mexiletine compound, wherein the composition does not include at least one ingredient present in NaMuscla®, such as, e.g., does not comprise one or more, two or more, three or more, four or more, or, e.g., 5 or more ingredients present in NaMuscla®. “NaMuscla®” or “approved immediate release mexiletine composition” here or elsewhere herein refers to the pharmaceutical product approved by the European Medicines Agency product number EMA/831802/2018, having received such first granted marketing authorization valid throughout the European Union on 21 Jul. 2016; the product was designated as an orphan medicinal product EU/3/14/1353 on 19 Nov. 2014; the product currently marked in Europe as NaMuscla®, or any combination thereof.

In one embodiment, the present invention provides a controlled release pharmaceutical composition administered no more than once per about 12-hour, 14-hour, 16-hour, 18-hour, 20-hour, 22-hour, or 24-hour period comprising a drug, e.g., a mexiletine compound, wherein said composition does not include at least one ingredient in Mexitil, such as, e.g., does not comprise one or more, two or more, three or more, four or more, or, e.g., 5 or more ingredients present in Mexitil. “Mexitil” or “approved commercial mexiletine hydrochloride product” here or elsewhere herein refers to the pharmaceutical product approved by the FDA under NDA #018873 and the trademarked Mexitil® and sold under such registered trademark in the United States prior to the submission of this disclosure, e.g., as of Dec. 31, 1985, Dec. 31, 2020, or Dec. 31, 2021, held by Boehringer Ingelheim Pharmaceuticals, Inc; initially approved on Dec. 30, 1985, or another product sold under the same NDA.

Exemplary target composition (e.g., product) profile characteristics are provided in Table 2 (below). In aspects, composition(s) provided herein comprise one or more, two or more, three or more four or more, or all five such characteristics provided in Table 2. In aspects, Table 2 provides one or more exemplary extended-release characteristics of compositions provided by the invention, one or more exemplary pharmacokinetic characteristics of compositions provided by the invention, or combinations thereof.

TABLE 2
Target Product Profile.

| TPP elements | Target |
|---|---|
| (a) Comparator | NaMuscla ® Cap 167 mg (Lupin) TID |
| (b) Type of PK study | Fasted single dose; food effect & steady state |
| (c) Oral BA | 80-90% Oral BA over 24-hour QD/12-hour BID relative to immediate |
| | release NaMuscla ® Capsule 200 mg administered TID; |
| | Objective to see: |
| | Comparable AUC over 24 hours |
| | Cₘᵢₙ at 24 hours >0.5 μg/mL or, >Cₘᵢₙ of IR dosing at steady state |
| | Cₘₐₓ ss: 80-120% of Cₘₐₓ of IR dosing at steady state |
| | Cₘₐₓ/Cₘᵢₙ ratio at steady state as low as possible |
| | At steady state - blood levels to be between 0.5 to 2 μg/mL |
| (d) Tmax | Suitable to provide 24-hour coverage (based on QD/BID dosing) and |
| | relative BA of 80-90% with respect to. TID administration of NaMuscla ® |
| | Cap 167 mg. |
| Stability and shelf | 90-110% of the label claim after 6 months of storage at 40° C./75% RH |
| life | providing a predicted shelf life of 18-24 months at long-term storage |
| | condition. (stability testing for assay, RS, water content & dissolution) |

In one aspect, the invention provides a pharmaceutical composition comprising a mexiletine compound complexed with a pharmaceutically acceptable ion exchange resin wherein the composition is characterizable as a controlled-release mexiletine compound composition, an extended-release mexiletine compound composition, or both. An immediate release mexiletine composition, such as, e.g., NaMuscla®, typically provides a therapeutic effect lasting between about 6-8 hours, such as, e.g., no more than about 6, no more than about 7, or, e.g., typically no more than about 8 hours. Thus, in aspects, an extended-release mexiletine compound composition or a controlled-release mexiletine compound composition described herein is characterized by having therapeutically effective plasma levels of mexiletine for at least about 10 to at least about 24 hours, such as, e.g., ≥˜12 hours, ≥˜14 hours, ≥˜16 hours, ≥˜18 hours, ≥˜20 hours, ≥˜22 hours, or, e.g., ≥˜24 hours. In aspects, release characteristics of compositions provided herein allow for administering compositions no more than about twice or no more than about once, e.g., only once or twice per day. In aspects, compositions herein are administered no more than once per day.

### Method of Use

In aspects, the invention provides a method for modulating sodium channels. In aspects, the invention blocks state-dependent (inactivated sodium channels). In aspects, the invention provides a method for modulating sodium channels, wherein sodium channels exhibit a detectably or significantly slower recovery time when compared to lidocaine modulation of sodium channels. In aspects, the invention provides methods of modulating sodium channels by enhancing the fast-inactivation of the sodium channels, resulting in use-dependent sodium channel blockage. In aspects, the invention provides methods of modulating sodium channels via any one of the aforementioned effects while also not demonstrating any noticeable, e.g., detectable or significant effect on chloride channels. In aspects, methods provided here can comprise the use of any one or more compositions described herein. In aspects, methods provided here can comprise the use of any one or more compositions described herein produced by any one or more methods described herein.

### Adverse Events

As previously noted above, compositions described herein can detectably or significantly reduce, limit, or, e.g., eliminate undesirable side effect(s) associated with multiple dosing of mexiletine immediate release compositions which include cardiac and GI-related undesirable side effects (such as, e.g., those reported related to use of NaMuscla®, e.g., reported in one or more NaMuscla® regulatory documents, product labels, or one or more appropriately controlled studies).

In aspects, compositions described herein detectably or significantly reduce one or more side effects associated with the administration of multiple doses of mexiletine immediate release compositions, such as, e.g., detectably or significantly reducing one or more cardiac-related effects, detectably or significantly reducing one or more GI related effects, or both.

In aspects, compositions described herein may exhibit detectably lower, or significantly less adverse events, specifically relating to cardiac and gastrointestinal adverse events, when compared to current on-market treatment, (e.g., treatment with NaMuscla®), which is administered three times a day.

### Compliance

In aspects, compositions described herein detectably or significantly improve patient compliance compared to mexiletine compound compositions requiring two or more or three or more doses per day, or e.g., per 12-hour, per 18-hour, or per 24-hour period.

In aspects, compositions described herein detectably or significantly improve patient compliance at an individual level, a population level, or both. In aspects, at a population level, e.g., within a population of myotonia patients, compositions described herein demonstrate detectably or significantly greater medication compliance due to single dosing of mexiletine compound(s), associated reduction(s) in one or more adverse cardiac events, associated reduction(s) in one or more GI related adverse events, or combination(s) thereof.

### Method of Production

In one aspect, the present invention provides a method of preparing a controlled release once daily dispersible tablet comprising mexiletine compound(s) and a pharmaceutically acceptable ion-exchange resin in the form of mexiletine compound-resin complex(es) and at least one release controlling coating layer that envelopes at least a portion of each complex, at least a portion of all complexes present in the composition, or both, to control release of mexiletine compound(s).

In aspects, the invention provides method(s) of manufacturing compositions described herein. In aspects, the invention provides a method (e.g., a process) of manufacturing wherein ion exchange resin particles are loaded with pharmaceutical drugs prior to incorporation in a polymeric system to form a drug resin complex, which is further coated by release retarding polymer to form a controlled release drug resin complex.

In aspects, the invention provides a product made by any process described herein, such as, e.g., method(s) of production described here.

### EXAMPLES OF APPLICATIONS, EMBODIMENTS, OR PRINCIPLES

The following detailed Examples of applications, embodiments, or principles (“Examples”) are provided to assist readers in further understanding aspects of the invention or principles related to practicing aspects of the invention. Any particular materials, methods, steps, and conditions employed/described in the following Examples, and any results thereof, are merely intended to further illustrate aspects of the invention. These Examples reflect exemplary embodiments of the invention, and the specific methods, findings, principles of such Examples, and the general implications thereof, can be combined with any other part of this disclosure. However, readers should understand that the invention is not limited by these Examples or any part thereof.

### Example 1: Process for Preparing Uncoated Mexiletine Ion Exchange Resin Complex

The following non-limiting example demonstrates a preferred composition of the present invention. Said examples were prepared on a weight-to-volume (w/v) basis.

The mexiletine ion exchange resin complex was prepared by first dissolving mexiletine hydrochloride in purified water under stirring. Next, sodium polystyrene sulfonate (Amberlite IRP 69) was dispersed into the solution by continuously stirring for 18 hours to 20 hours. Then the dispersion was allowed to settle after stirring to decant the supernatant. After decanting the supernatant, enough water was added while stirring continuously for a suitable period of time to form a slurry. The slurry was filtered through 325 mesh and air dried in a fluidized bed dryer for 30 min followed by drying at an inlet temperature of 60° C. till LOD NMT 4.0% is obtained to finally form the mexiletine ion exchange resin complex.

The Mexiletine ion exchange resin ratio and percentage of mexiletine loading on ion exchange resin for Compositions A, B, C, and D are presented in Table 3 (below).

TABLE 3
Mexiletine ion exchange resin ratio and percentage
of mexiletine loading on ion exchange resin.

| | Composition | | | |
|---|---|---|---|---|
| | A | B | C | D |
| | Drug Resin Ratio | | | |
| | 1:0.5 | 1:1 | 1:2 | 1:5 |
| | % w/w | % w/w | % w/w | % w/w |
| Mexiletine hydrochloride | 66.67 | 50 | 33.33 | 16.67 |
| Potassium Polystyrene Sulfonate | 33.33 | 50 | 66.67 | 83.33 |
| (Amberlite IRP 69) | | | | |
| Purified water | q.s | q.s | q.s | q.s |
| Total | 100 | 100 | 100 | 100 |
| % Drug Loading | 200 | 100 | 50 | 20 |

A dissolution study was performed for the exemplary uncoated mexiletine ion exchange resin complex in a USP Type IV dissolution device, 8 mL/min, 6 g Beads, 0.1N HCl, at 37° C.±0.5° C., the data for which is presented in Table 4 (below). The basic conditions for the dissolution study comprise those conditions known in the art.

TABLE 4
Dissolution study for uncoated mexiletine ion exchange resin
complex in USP Type IV, 8 mL/min, 6 g Beads, 0.1N HCl,
37° C. ± 0.5° C.

Composition

| | A | B | C | D |
|---|---|---|---|---|
| Time (h) | % CDR | % CDR | % CDR | % CDR |
| 0 | 0 | 0 | 0 | 0 |
| 0.5 | 27 | 33 | 17 | 9 |
| 1 | 42 | 52 | 32 | 15 |
| 2 | 57 | 69 | 50 | 26 |
| 3 | 65 | 79 | 62 | 37 |
| 4 | 70 | 87 | 72 | 46 |
| 5 | 73 | 91 | 79 | 54 |
| 6 | 75 | 95 | 84 | 61 |
| 8 | 78 | 99 | 91 | 71 |
| 10 | 80 | 102 | 96 | 79 |
| 12 | 81 | 104 | 98 | 84 |

* % CDR: % cumulative drug release

As shown in Table 4 (above), Composition B and Composition D demonstrate a dissolution profile wherein, on average, the drug resin ratio compositions are 1:1 and 1:15, respectively. As further shown in Table 4 (above), Compositions A and Composition D demonstrate a dissolution profile wherein, on average, about 81% and 84%, respectively, of the total respective Composition dissolved after 12 hours under the tested conditions. These results exemplify that compositions according to the invention can exhibit a dissolution profile under which a significant amount of mexiletine remains undissolved after a number of hours (at least about 15% remaining, such as 10%-50% remaining, such as about 5% to about 40% remaining, after 6 hours, at least 1% remaining, such as 1% to 30% remaining, and in most cases at least about 10%, after 8 hours; at least about 1% in most cases, such as 2% to 20%, after 12 hours).

An assay of compositions A, B, C, and D was performed, the data for which is presented in Table 5 (below). The basic conditions for the assay comprise testing carried out by HPLC equipped with a UV/PDA detector.

TABLE 5
% Assay of compositions A, B, C, and D.

| Composition | A | B | C | D |
|---|---|---|---|---|
| % Assay | 82.2 | 101.7 | 103.4 | 95.2 |

A second dissolution study was performed for the exemplary uncoated mexiletine ion exchange resin complex in USP Type IV, 8 mL/min, 6 g Beads, and pH 5.0 acetate buffer media at 37° C.±0.5° C., the data for which is presented in Table 6 (below).

TABLE 6
Dissolution study for uncoated mexiletine ion exchange
resin complex in USP Type IV, 8 mL/min, 6 g Beads, pH 5.0
Acetate buffer media, at 37° C. ± 0.5° C.
Composition B

| Time (h) | % CDR |
|---|---|
| 0 | 0 |
| 0.5 | 16 |
| 1 | 28 |
| 2 | 46 |
| 3 | 59 |
| 4 | 68 |
| 5 | 75 |
| 6 | 80 |
| 8 | 88 |
| 10 | 93 |
| 12 | 96 |

* % CDR: % cumulative drug release

As shown in Table 6 (above), Composition B demonstrates a dissolution profile wherein, on average, about 88% of the total Composition dissolved after 8 hours at a pH of 5.0 under the tested conditions. These results exemplify that compositions according to the invention can exhibit a dissolution profile under which a significant amount of mexiletine remains undissolved after a number of hours (at least about 20% remaining, after 6 hours, at least 12% remaining, after 8 hours; at least about 4% remaining, after 12 hours).

A third dissolution study was performed for the exemplary uncoated mexiletine ion exchange resin complex in USP Type IV, 8 mL/min, 6 g Beads, and pH 6.8 phosphate buffer media at 37° C.±0.5° C., the data for which is presented in Table 7 (below).

TABLE 7
Dissolution study for uncoated mexiletine ion exchange
resin complex in USP Type IV, 8 mL/min, 6 g Beads,
pH 6.8 phosphate buffer media, at 37° C. ± 0.5° C.

| Time (h) | Composition B % CDR |
|---|---|
| 0 | 0 |
| 0.5 | 34 |
| 1 | 57 |
| 2 | 81 |
| 3 | 91 |
| 4 | 96 |
| 5 | 99 |
| 6 | 100 |
| 8 | 100 |
| 10 | 100 |
| 12 | 100 |

* % CDR: % cumulative drug release

As shown in Table 7 (above), Composition B demonstrates a dissolution profile wherein, on average, about 96% of the total Composition dissolved after 4 hours at a pH of 6.8 under the tested conditions. These results exemplify that compositions according to the invention can exhibit a dissolution profile under which a significant amount of mexiletine remains undissolved after a number of hours (at least about 20% remaining, after 2 hours, at least 10% remaining, after 3 hours; at least about 4% remaining, after 4 hours).

### Example 2: Process for Extended-Release Coating of Mexiletine Ion Exchange Resin Complex Using Fluidized Bed Processor

This Example demonstrates a process for extended-release coating of the exemplary mexiletine ion exchange resin complex using a fluidized bed processer.

Step 1: Hydroxypropyl methyl cellulose was dissolved in a sufficient quantity of water and/or isopropyl alcohol while stirring continuously.

Step 2: Mexiletine ion exchange resin was loaded in a fluidized bed processor and coating solution was applied until a desired weight was achieved.

Step 3: Further, hydroxy propyl methyl cellulose was dispersed in isopropyl alcohol under stirring and methylene dichloride was added and stirred continuously until a clear solution was achieved.

Step 4: Ethyl cellulose and triethyl citrate were added to the above solution and stirred until a clear solution was formed.

Step 5: The coated mexiletine ion exchange resin complex obtained from step 2 was again coated with the coating solution of step 4 in a fluidized bed processor until a desired coating weight was achieved. Finally, the coated mexiletine ion exchange resin complex was dried until LOD was not more than 4.0% and sized through a suitable sieve or screen.

Coating concentrations to provide extended-release mexiletine ion exchange resin complex are presented in Table 8 (below).

TABLE 8
Coating concentrations to provide extended-release
mexiletine ion exchange resin complex.

| | Composition | | | | |
|---|---|---|---|---|---|
| | E | F | G | H | I |
| | % w/w | % w/w | % w/w | % w/w | % w/w |
| Drug - Resin Complex | 93.37 | 89.13 | 93.37 | 89.13 | 87.54 |
| Coat 1 | | | | | |
| Hydroxy propyl | 1.87 | 1.78 | 1.87 | 1.78 | 1.75 |
| methylcellulose | | | | | |
| (3 mPa to 4000 mPa) | | | | | |
| IPA | q.s | q.s | q.s | q.s | q.s |
| Water | q.s | q.s | q.s | q.s | q.s |
| Coat 2 | | | | | |
| Hydroxy propyl | 1.13 | 2.16 | 0.45 | 0.86 | 2.54 |
| methyl cellulose | | | | | |
| (3 mPa to 4000 mPa) | | | | | |
| Ethyl Cellulose | 3.39 | 6.48 | 4.07 | 7.77 | 7.63 |
| (3 mPa to 330 mPa) | | | | | |
| Triethyl Citrate | 0.24 | 0.45 | 0.24 | 0.45 | 0.54 |
| IPA | q.s | q.s | q.s | q.s | q.s |
| DCM | q.s | q.s | q.s | q.s | q.s |
| Total | 100 | 100 | 100 | 100 | 100 |

A dissolution study was performed for coated drug resin complex in USP Type IV, 8 mL/min, 6 g Beads, 0.1N HCl at 37° C.±0.5° C., the data for which is presented in Table 9 (below).

TABLE 9
Dissolution study for coated drug resin complex
in USP Type IV, 8 mL/min, 6 g Beads, 0.1N
HCl, temp 37° C. ± 0.5° C.

| | Composition | | | | |
|---|---|---|---|---|---|
| | E | F | G | H | I |
| | % Assay | | | | |
| | — | 102.8 | — | 99.7 | — |
| Time (h) | % CDR | % CDR | % CDR | % CDR | % CDR |
| 0 | 0 | 0 | 0 | 0 | 0 |
| 0.5 | 24 | 19 | 12 | 11 | 8 |
| 1 | 40 | 34 | 24 | 21 | 16 |
| 2 | 55 | 58 | 42 | 36 | 27 |
| 3 | 63 | 72 | 54 | 46 | 35 |
| 4 | 69 | 80 | 63 | 54 | 41 |
| 5 | 74 | 84 | 69 | 61 | 46 |
| 6 | 78 | 88 | 73 | 66 | 51 |
| 8 | 83 | 92 | 79 | 73 | 57 |
| 10 | 87 | 95 | 83 | 78 | 61 |
| 12 | 90 | 97 | 86 | 82 | 65 |

* % CDR: % cumulative drug release

As shown in Table 9 (above), Composition E and Composition F demonstrate a dissolution profile wherein, on average, about 83% and 92%, respectively, of the total Compositions are dissolved after 8 hours under the tested conditions. As further shown in Table 9, Composition G and Composition H demonstrate a dissolution profile wherein, on average, about 79% and 73%, respectively, of the total Compositions are dissolved after 8 hours under the tested conditions. Further, as shown in Table 9, Composition I demonstrates a dissolution profile wherein, on average, about 57% of the total Composition is dissolved after 8 hours under the tested conditions. Notably, as shown by Table 9, Composition I demonstrates a dissolution profile wherein, on average, about 65% of the total Composition is dissolved after 12 hours under the tested conditions. These results exemplify that compositions according to the invention can exhibit a dissolution profile under which a significant amount of mexiletine remains undissolved after a number of hours (at least about 35% remaining, such as about 16% to about 50% remaining, after 6 hours, at least 25% remaining, such as 10% to 40% remaining, and in most cases at least about 30%, after 8 hours; at least about 15% in most cases, such as 5% to 35%, after 12 hours).

A second dissolution study was performed for coated drug resin complex in USP Type IV, 8 mL/min, 6 g Beads, pH 5.0 acetate buffer media at 37° C.±0.5° C., the data for which is presented in Table 10 (below).

TABLE 10
Dissolution study for coated drug resin complex
in USP Type IV, 8 mL/min, 6 g Beads, pH 5.0
Acetate buffer media, temp 37° C. ± 0.5° C.

| | Composition F | Composition H |
|---|---|---|
| Time (h) | % CDR | % CDR |
| 0 | 0 | 0 |
| 0.5 | 12 | 7 |
| 1 | 20 | 13 |
| 2 | 33 | 21 |
| 3 | 42 | 28 |
| 4 | 50 | 34 |
| 5 | 56 | 38 |
| 6 | 60 | 42 |
| 8 | 67 | 49 |
| 10 | 72 | 54 |
| 12 | 77 | 59 |

* % CDR: % cumulative drug release

As shown in Table 10 (above), Composition F and Composition H demonstrate a dissolution profile wherein, on average, about 67% and 49%, respectively, of the total Compositions, are dissolved after 8 hours at a pH of 5.0 under the tested conditions. As further shown in Table 10, Composition F and Composition H demonstrate a dissolution profile wherein, on average, about 77% and 59%, respectively, of the total Compositions are dissolved after 12 hours at a pH of 5.0 under the tested conditions. These results exemplify that compositions according to the invention can exhibit a dissolution profile under which a significant amount of mexiletine remains undissolved after a number of hours (at least about 45% remaining, such as about 40% to about 58% remaining, after 6 hours, at least 40% remaining, such as 30% to 50% remaining, after 8 hours; at least about 35% in most cases, such as 35% to 45%, after 12 hours).

A third dissolution study was performed for coated drug resin complex in USP Type IV, 8 mL/min, 6 g Beads, pH 6.8 Phosphate buffer media at 37° C.±0.5° C., the data for which is presented in Table 11 (below).

TABLE 11
Dissolution study for coated drug resin complex
in USP Type IV, 8 mL/min, 6 g Beads, pH 6.8
phosphate buffer media, temp 37° C. ± 0.5° C.

| | Composition F | Composition H |
|---|---|---|
| Time (h) | % CDR | % CDR |
| 0 | 0 | 0 |
| 0.5 | 19 | 13 |
| 1 | 32 | 22 |
| 2 | 48 | 33 |
| 3 | 58 | 41 |
| 4 | 65 | 48 |
| 5 | 70 | 53 |
| 6 | 74 | 57 |
| 8 | 80 | 64 |
| 10 | 85 | 69 |
| 12 | 88 | 73 |

* % CDR: % cumulative drug release

As shown in Table 11 (above), Composition F and Composition H demonstrate a dissolution profile wherein, on average, about 80% and 64%, respectively, of the total Compositions, are dissolved after 8 hours at a pH of 6.8 under the tested conditions. As further shown in Table 11, Composition F and Composition H demonstrate a dissolution profile wherein, on average, about 88% and 73%, respectively, of the total Compositions, are dissolved after 12 hours at a pH of 6.8 under the tested conditions. These results exemplify that compositions according to the invention can exhibit a dissolution profile under which a significant amount of mexiletine remains undissolved after a number of hours (at least about 35% remaining, such as about 30% to about 45% remaining, after 6 hours, at least 30% remaining, such as 20% to 35% remaining, after 8 hours; at least about 20% in most cases, such as 15% to 25%, after 12 hours).

### Example 3: Process of Making Dry Powder for Oral Suspension

This Example demonstrates a process for making dry powder for oral suspension.

Step 1: Sucrose (part quantity) was mixed with a suspending agent and a viscosity modifier (hydroxy propyl cellulose or spray-dried blend of microcrystalline cellulose and carboxymethyl cellulose sodium or guar gum or xanthan gum or sodium carboxymethyl cellulose or povidone) and sift through 40 mesh sieve.

Step 2: Methyl paraben, propyl paraben, and part of the quantity of sucrose were sifted together through a 40-mesh sieve.

Step 3: Colloidal silicon dioxide or microcrystalline cellulose or starch or talc or sucralose or aspartame was co-sifted through a 40 mesh sieve with part quantity of sucrose.

Step 4: Further, flavor with part quantity of sucrose was co-sifted through a 40 mesh sieve.

Step 5: Contents of step 1 were mixed with step 4.

Step 6: Coated mexiletine ion exchange resin complex was added to step 5 and blended.

Step 7: Contents of step 6 were filled in sachets (unit dose or multiple dose) or filled in bottles (unit dose or multiple dose).

### Example 4: Process for Making Constituted Oral Suspension

This Example demonstrates a process for making constituted oral suspension.

Microcrystalline cellulose and carboxymethyl cellulose sodium co-mix as a suspending agent was added to water under stirring, and then sucrose was added under stirring. preservative, flavor, and sweeteners were added to it subsequently. The coated drug resin complex as obtained in example 2 was added to it and stirred. The final content was filled in sachets or bottles (unit dose or multiple dose).

The ingredients for the suspension composition 1 (TI) and respective % w/w are presented in Table 12 (below).

TABLE 12
Suspension composition 1 (T1).

| Ingredients | % w/w |
|---|---|
| Drug-Resin Complex | 45.20 |
| Coat 1 | |
| Hydroxy propyl methyl Cellulose | 0.90 |
| IPA | q.s. |
| Water | q.s. |
| Coat 2 | |
| Hydroxy propyl methyl Cellulose | 1.09 |
| Ethyl Cellulose (7 Cps) | 3.28 |
| Triethyl Citrate | 0.23 |
| IPA | q.s. |
| DCM | q.s. |
| Suspension Base | |
| Sodium CMC-MCC Co-mix | 2.82 |
| Sucrose | 16.95 |
| Maize starch | 9.04 |
| Hydroxypropyl Cellulose | 4.52 |
| Sucrose | 11.10 |
| Sucralose | 1.69 |
| Strawberry Flavour | 1.13 |
| Col. Silicon Dioxide | 1.69 |
| Methyl paraben | 0.28 |
| Propyl paraben | 0.06 |
| Total | 100.00 |

A dissolution study was performed for suspension composition 1 (TI) in USP Type IV, 8 mL/min, 6 g Beads, and 0.1N HCl at 37° C.±0.5° C. the data for which is presented in Table 13 (below).

TABLE 13
Dissolution study for suspension composition
1 (T1) in USP Type IV, 8 mL/min, 6 g Beads,
0.1N HCl, 37° C. ± 0.5° C.

| Time (Hour) | % CDR |
|---|---|
| 0 | 0 |
| 0.5 | 10 |
| 1 | 18 |
| 2 | 30 |
| 3 | 38 |
| 4 | 44 |
| 5 | 49 |
| 6 | 53 |
| 8 | 59 |
| 10 | 64 |
| 12 | 67 |
| 14 | 70 |
| 16 | 73 |
| 18 | 75 |
| 20 | 77 |
| 24 | 81 |

* % CDR: % cumulative drug release

As shown in Table 13 (above), the suspension composition 1 (T1) demonstrates a dissolution profile wherein, on average, about 59% of the total suspension composition is dissolved after 8 hours under the tested conditions. As further shown in Table 13, the suspension composition demonstrates a dissolution profile wherein, on average, about 81% of the total suspension composition is dissolved after 24 hours under the tested conditions. These results exemplify that compositions according to the invention can exhibit a dissolution profile under which a significant amount of mexiletine remains undissolved after a number of hours (at least about 50% remaining, after 6 hours, at least 30% remaining, after 16 hours; at least about 20% remaining, after 24 hours).

As further exemplified by the results of this study, and those above, this invention provides various forms and methods for making the composition which may likely be advantageous to the treatment population.

### Example 5: Stability Studies

This Example demonstrates a number of stability studies.

Stability Study for suspension composition 1 (TI): Samples were kept for stability study for a period of 1 month, 3 months, and 6 months. Following which percent (%) assay and degradation products study were performed to assess the stability of samples. The details of the stability study are provided below for 40° C./75% RH and 30° C./75% RH conditions.

A stability study was performed for 40° C./75% RH with Alu-Sachet, the data for which is presented in Table 14 (below). The basic conditions for the stability study comprise those conditions well known in the art.

TABLE 14
Stability study- 40° C./75% RH with Alu-Sachet.

| Test | Initial | 1 month | 3 month | 6 month |
|---|---|---|---|---|
| Assay % | 102.8 | 104.9 | 102.7 | 99.3 |

Degradation Products

| | | | | |
|---|---|---|---|---|
| KOE 5692 CL | 0.01 | 0.01 | 0.01 | 0.01 |
| Single maximum | Not | Not | Not | Not |
| unknown impurity | Detected | Detected | Detected | Detected |
| “Total degradation | 0.01 | 0.01 | 0.01 | 0.01 |
| products” | | | | |

The results above demonstrate that formulations of the invention are stable over significant periods of time under these conditions. From the data, a person of ordinary skill in the art could find a minimal loss of less than about 5%, such as less than about 1%.

A second stability study was performed for 30° C./75% RH with Alu-Sachet, the data for which is presented in Table 15 (below).

TABLE 15
Stability study- 30° C./75% RH with Alu-Sachet.

| Test | Initial | 3 month | 6 month |
|---|---|---|---|
| Assay % | 102.8 | 100.4 | 101.2 |

Degradation Products

| | | | |
|---|---|---|---|
| KOE 5692 CL | 0.01 | 0.01 | 0.01 |
| Single maximum | Not | Not | Not |
| unknown impurity | Detected | Detected | Detected |
| “Total degradation | 0.01 | 0.01 | 0.01 |
| products” | | | |

The results above demonstrate that formulations of the invention are stable over significant periods of time under these conditions. From the data, a person of ordinary skill in the art could find a minimal loss of less than about 5%, such as less than about 1%.

A third stability study was performed for 40° C./75% RH with glass vial(s), the data for which is presented in Table 16 (below).

TABLE 16
Stability study- 40° C./75% RH with Glass Vial.

| Test | Initial | 1 month | 3 month | 6 month |
|---|---|---|---|---|
| Assay % | 102.8 | 99.4 | 101 | 100 |

Degradation Products

| | | | | |
|---|---|---|---|---|
| KOE 5692 CL | 0.01 | 0.01 | 0.01 | 0.01 |
| Single maximum | Not | Not | Not | Not |
| unknown impurity | Detected | Detected | Detected | Detected |
| “Total degradation | 0.01 | 0.01 | 0.01 | 0.01 |
| products” | | | | |

The results above demonstrate that formulations of the invention are stable over significant periods of time under these conditions. From the data, a person of ordinary skill in the art could find a minimal loss of less than about 5%, such as less than about 1%.

A fourth stability study was performed for 30° C./75% RH with glass vials, the data for which is presented in Table 17 (below).

TABLE 17
Stability study-30° C./75% RH with Glass Vial.

| Test | Initial | 3 month | 6 month |
|---|---|---|---|
| Assay % | 102.8 | 100.8 | 98.6 |

Degradation Products

| | | | |
|---|---|---|---|
| KOE 5692 CL | 0.01 | 0.01 | 0.01 |
| Single maximum | Not | Not | Not |
| unknown impurity | Detected | Detected | Detected |
| “Total degradation | 0.01 | 0.01 | 0.01 |
| products” | | | |

The results above demonstrate that formulations of the invention are stable over significant periods of time under these conditions. From the data, a person of ordinary skill in the art could find a minimal loss of less than about 5%, such as less than about 1%.

The ingredients for the suspension composition 2 (T2) and respective % w/w are presented in Table 18 (below).

TABLE 18
Suspension composition 2 (T2).

| Ingredients | % w/w |
|---|---|
| Drug-Resin Complex | 45.20 |
| Coat 1 | |
| Hydroxypropyl methyl cellulose | 0.90 |
| IPA | q.s. |
| Water | q.s. |
| Coat 2 | |
| HPMC E 5LV | 0.44 |
| Ethyl Cellulose (7 Cps) | 3.94 |
| Triethyl Citrate | 0.23 |
| IPA | q.s. |
| DCM | q.s. |
| Suspension Base | |
| Sodium CMC-MCC Co-mix | 2.82 |
| Sucrose (300 Mesh pass) | 16.95 |
| Maize starch | 9.04 |
| Hydroxypropyl cellulose | 4.52 |
| Sucrose | 11.10 |
| Sucralose | 1.69 |
| Strawberry Flavour | 1.13 |
| Col. Silicon Dioxide | 1.69 |
| Methyl paraben | 0.28 |
| Propyl paraben | 0.06 |
| Total | 100.00 |

### Example 6: Dissolution Studies

This Example demonstrates a number of dissolution studies.

A dissolution study was performed for suspension composition 2 (T2) in USP Type IV, 8 mL/min, 6 g Beads at 37° C.±0.5° C., the data for which is present in Table 19 (below).

TABLE 19
Dissolution study for suspension composition 2 (T2) in USP Type
IV, 8 mL/min, 6 g Beads, 37° C. ± 0.5° C., 0.1N
HCl; pH 5.0 Acetate Buffer and pH 6.8 Phosphate Buffer (multimedia).

| | | % CDR in pH | % CDR in pH |
|---|---|---|---|
| | % CDR in 0.1N | 5.0 Acetate | 6.8 Phosphate |
| Time (Hour) | HCl | buffer | Buffer |
| 0 | 0 | 0 | 0 |
| 0.5 | 8 | 9 | 14 |
| 1 | 14 | 15 | 23 |
| 2 | 24 | 23 | 35 |
| 3 | 31 | 30 | 44 |
| 4 | 37 | 36 | 51 |
| 5 | 41 | 41 | 56 |
| 6 | 45 | 46 | 61 |
| 8 | 51 | 53 | 68 |
| 10 | 56 | 60 | 72 |
| 12 | 59 | 65 | 76 |
| 14 | 63 | — | — |
| 16 | 65 | — | — |
| 18 | 68 | — | — |
| 20 | 70 | — | — |
| 24 | 74 | — | — |

* % CDR: % cumulative drug release

As shown in Table 19 (above), the suspension composition 2 (T2) demonstrates a dissolution profile wherein, on average, about 51% of the total suspension composition is dissolved after 8 hours in 0.1N HCl under the tested conditions. As further shown in Table 19, the suspension composition 2 (T2) demonstrates a dissolution profile wherein, on average, about 53% of the total suspension composition is dissolved after 8 hours at pH 5.0 under the tested conditions. As further shown in Table 19, the suspension composition 2 (T2) demonstrates a dissolution profile wherein, on average, about 68% of the total suspension composition is dissolved after 8 hours at pH 6.8 under the tested conditions. These results exemplify that compositions according to the invention can exhibit a dissolution profile under which a significant amount of mexiletine remains undissolved after a number of hours (at least about 50% remaining, such as about 40% to about 65% remaining, after 6 hours, at least 45% remaining, such as 40% to 50% remaining, after 8 hours; at least about 30% in most cases, such as 25% to 40%, after 12 hours).

A dissolution study for compositions 1 and 2 (T1 and T2) was performed in 500 mL 0.1N HCl for the initial one (1) hour, followed by one (1) hour in 700 mL 4.5 pH acetate buffer changeover media, then two (2) hours in 900 mL phosphate buffer pH 6.8 changeover media by using USP type II (paddle), the data for which is present in Table 20 (below).

TABLE 20
Dissolution study for composition 1 and 2 (T1 and T2) in 500
mL 0.1N HCl for an initial 1 hour followed by 1 hour in 700
mL 4.5 pH acetate buffer changeover media to 2 hours in 900
mL phosphate buffer pH 6.8 changeover media by using USP
type II (paddle), 75 rpm at temp. 37° C. ± 0.5° C.

| | |
|---|---|
| Time | % CDR |

| Dissolution media | (hour) | T1 | T2 |
|---|---|---|---|
| 0.1N HCl | 1 | 38 | 30 |
| pH 4.5 acetate buffer | 1 | 88 | 81 |
| pH 6.8 phosphate buffer | 2 | 99 | 98 |

* % CDR: % cumulative drug release

As shown in Table 20 (above), the suspension composition 1 (T1) demonstrates a dissolution profile wherein, on average, about 38% of the total suspension composition is dissolved after 1 hour in 0.1N HCl under the tested conditions. As further shown in Table 20, the suspension composition 1 (T1) demonstrates a dissolution profile wherein, on average, about 88% of the total suspension composition is dissolved at pH 4.5 under the tested conditions. As further shown in Table 20, the suspension composition 1 (T1) demonstrates a dissolution profile wherein, on average, about 99% of the total suspension composition is dissolved at pH 6.8 under the tested conditions. As also shown in Table 20, the suspension composition 2 (T2) demonstrates a slightly more advantageous dissolution profile, wherein, on average, about 30%, about 81%, and about 98% of the total suspension composition is dissolved after the three tested conditions are incrementally applied. These results exemplify that compositions according to the invention can exhibit a dissolution profile under which a significant amount of mexiletine remains undissolved after a number of hours (at least about 65% remaining, such as about 62% to about 70% remaining, after the initial 1 hour, at least 15% remaining, such as 10% to 20% remaining, after the additional 1 hour at pH 4.5; at least about 1.5% in most cases, such as 1% to 2%, after the final 2 hours at pH 6.8).

### Example 7: Suspension Study for Composition T2

This Example demonstrates a suspension study for composition T2.

Stability study for suspension composition 2 (T2): samples were kept for stability study for a period of 1 month and 3 months. Following which percent (%) assay by HPLC method and degradation products study were performed to assess the stability of samples. The details of the stability study are provided below for 40° C./75% RH conditions.

A stability study of composition 2 (T2) was performed at 40° C./75% RH conditions, the data for which is presented in Table 21 (below).

TABLE 21
Stability data of composition 2 (T2) at 40° C./75% RH conditions.

| Test | Initial | 1 month | 3 month |
|---|---|---|---|
| Assay % | 102 | 104.4 | 102.2 |

Degradation products

| | | | |
|---|---|---|---|
| KOE 5692 CL | 0.01 | 0.01 | 0.01 |
| Single maximum | Not | Not | Not |
| unknown impurity | Detected | Detected | Detected |
| Total degradation | 0.01 | 0.01 | 0.01 |
| products | | | |

The results above demonstrate that formulations of the invention are stable over significant periods of time under these conditions. From the data, a person of ordinary skill in the art could find a minimal loss of less than about 5%, such as less than about 1%.

### Example 8: Suspension Composition T3 and Dissolution Study

This Example demonstrates the ingredients of suspension composition 3 (T3) as well as a dissolution study performed on suspension composition T3.

The ingredients of suspension composition 3 (T3) are presented in Table 22 (below).

TABLE 22
Suspension composition 3 (T3).

| Ingredients | % w/w |
|---|---|
| Extended-Release Drug Resin Complex | 48.31 |
| Drug Resin Complex | 2.27 |
| Sodium CMC-MCC Co-mix | 2.83 |
| Sucrose | 17.00 |
| Maize starch | 9.06 |
| Hydroxyl propyl cellulose | 4.53 |
| Sucrose | 11.13 |
| Sucralose | 1.70 |
| Strawberry Flavour | 1.13 |
| Aerosil 200 | 1.70 |
| Methyl paraben | 0.28 |
| Propyl paraben | 0.06 |
| Total | 100.00 |

A dissolution study for suspension composition 3 (T3) was performed in 0.1 N HCl, Apparatus USP type IV, Flow rate 8 mL/min along with 6 g glass bead at 37° C.±0.5° C., the data for which is presented in Table 23 (below).

TABLE 23
Dissolution study for suspension composition 3 (T3)
in 0.1N HCl, Apparatus USP type IV, Flow rate 8 mL/min
along with 6 g glass bead, temp. 37° C. ± 0.5° C.

| Time (Hour) | % CDR |
|---|---|
| 0 | 0 |
| 0.5 | 11 |
| 1 | 19 |
| 2 | 32 |
| 3 | 42 |
| 4 | 49 |
| 5 | 56 |
| 6 | 61 |
| 8 | 70 |
| 10 | 76 |
| 12 | 81 |

* % CDR: % cumulative drug release

As shown in Table 23 (above), the suspension composition 3 (T3) demonstrates a dissolution profile wherein, on average, about 70% of the total suspension composition is dissolved after 8 hours under the tested conditions. As further shown in Table 23, the suspension composition 3 (T3) demonstrates a dissolution profile wherein, on average, about 81% of the total suspension composition is dissolved after 12 hours under the tested conditions. These results exemplify that compositions according to the invention can exhibit a dissolution profile under which a significant amount of mexiletine remains undissolved after a number of hours (at least about 40% remaining, after 6 hours, at least 30% remaining, after 8 hours; at least about 20% remaining, after 12 hours).

### Example 9: Dispersible Tablet Examples and Studies Therewith

This Example demonstrates the composition of dispersible tablets as well as their corresponding studies.

The ingredients for the Dispersible Tablets of composition 1 (D1) and composition 2 (D2) are presented in Table 24 (below).

TABLE 24
Dispersible Tablets composition 1 (D1) and composition 2 (D2).

Dispersible Tablets composition

| | |
|---|---|
| D1 | D2 |
| (EC:HPMC | (EC:HPMC |
| ratio is 75:25) | ratio is 90:10) |

| | |
|---|---|
| Ingredients | % w/w |

| | | |
|---|---|---|
| ER-coated Mexiletine HCl | 64.11 | 64.11 |
| Resinate | | |
| Crosspovidone | 4.29 | 4.29 |
| Microcrystalline cellulose | 13.17 | 13.17 |
| Col. Silicon Dioxide | 1.00 | 1.00 |
| Sucralose | 2.14 | 2.14 |
| Strawberry Flavour | 1.43 | 1.43 |
| Microcrystalline cellulose | 12.86 | 12.86 |
| Magnesium Stearate | 1.00 | 1.00 |
| Total | 100.00 | 100.00 |

A dissolution study for Dispersible Tablets of composition 1 (D1) and composition 2 (D2) was performed in 0.1 N HCl, Apparatus USP type IV, Flow rate 8 mL/min along with 6 g glass bead, at 37° C.±0.5° C., the data for which are presented in Table 25 (below).

TABLE 25
Dissolution study for Dispersible tablets compositions 1 and
2 (D1& D2) in 0.1N HCl, Apparatus USP type IV, Flow rate 8
mL/min along with 6 g glass bead, temp. 37° C. ± 0.5° C.

| Time (Hour) | % CDR: D1 | % CDR: D2 |
|---|---|---|
| 0 | 0 | 0 |
| 0.5 | 17 | 12 |
| 1 | 30 | 21 |
| 2 | 50 | 38 |
| 3 | 66 | 53 |
| 4 | 79 | 68 |
| 5 | 88 | 81 |
| 6 | 93 | 90 |
| 8 | 98 | 98 |
| 10 | 100 | 101 |
| 12 | 100 | 101 |

* % CDR: % cumulative drug release

As shown in Table 25 (above), the dispersible tablet compositions 1 and 2 (D1 & D2) demonstrate dissolution profiles wherein, on average, about 98% and 98%, respectively, of the total dispersible tablets are dissolved after 8 hours under the tested conditions. These results exemplify that compositions according to the invention can exhibit a dissolution profile under which a significant amount of mexiletine remains undissolved after a number of hours (at least about 25% remaining, such as about 20% to about 30% remaining, after 4 hours, at least 15% remaining, such as 10% to 20% remaining, after 5 hours; at least ˜2% remaining, after 8 hours).

A second set of dissolution studies for Dispersible Tablets of composition 1 (D1) and composition 2 (D2) was performed in 500 mL 0.1N HCl for the initial (1) hour, followed by (1) hour in 700 mL 4.5 pH acetate buffer changeover media, then to (2) hours in 900 mL phosphate buffer pH 6.8 changeover media by using USP type II (paddle), 75 rpm at 37° C.±0.5° C., the data for which are presented in Table 26 (below).

TABLE 26
Dissolution study for dispersible tablets 1 and 2 (D1 and D2)
in 500 mL 0.1N HCl for an initial 1 hour followed by 1 hour
in 700 mL 4.5 pH acetate buffer changeover media to 2 hours
in 900 mL phosphate buffer pH 6.8 changeover media by using
USP type II (paddle), 75 rpm at temp. 37° C. ± 0.5° C.

| | |
|---|---|
| Time | % CDR |

| Dissolution media | (hour) | D1 | D2 |
|---|---|---|---|
| 0.1N HCl | 1 | 40 | 44 |
| pH 4.5 acetate buffer | 1 | 81 | 83 |
| pH 6.8 phosphate buffer | 2 | 94 | 95 |

* % CDR: % cumulative drug release

As shown in Table 26 (above), the dispersible tablet compositions 1 and 2 (D1 & D2) demonstrate dissolution profiles wherein, on average, about 94% and 95%, respectively, of the total dispersible tablets are dissolved after the three tested conditions are applied. These results exemplify that compositions according to the invention can exhibit a dissolution profile under which a significant amount of mexiletine remains undissolved after a number of hours (at least about 68% remaining, such as about 55% to about 60% remaining, after 1 hour, at least 20% remaining, such as 7% to 20% remaining, after 1 hour at a pH 4.5; at least about 5% remaining, such as 5% to 6%, after 2 hours at pH 6.8).

Stability study for dispersible tablets composition 1 and 2 (D1 and D2): Samples were kept for stability study for a period of 6 months. Following which % assay by HPLC method and degradation products study were performed to assess the stability of samples. The details of the stability study are provided below for 40° C./75% RH conditions.

Stability studies for dispersible tablet compositions 1 and 2 (D1 and D2) were performed, the data for which are presented in Table 27 (below).

TABLE 27
Stability study for 40° C./75% RH conditions.

| | Compositions | | | |
|---|---|---|---|---|
| | Dispersible tablets (D1) | | Dispersible tablets (D2) | |
| | | 6 M - 40/75% | | 6 M - 40/75% |
| Test | Initial | RH | Initial | RH |
| Assay % | 97.7 | 97.7 | 96.7 | 97.7 |

Degradation Products

| | | | | |
|---|---|---|---|---|
| KOE 5692 CL | 0.02 | 0.02 | 0.02 | 0.02 |
| Single maximum | ND | ND | ND | ND |
| unknown impurity | | | | |
| Total degradation | 0.02 | 0.02 | 0.02 | 0.02 |
| products | | | | |

The results above demonstrate that formulations of the invention are stable over significant periods of time under these conditions. From the data, a person of ordinary skill in the art could find a minimal loss of less than about 1%.

### Example 10: Relative Bioavailability Study

### Fasting Study:

This Example demonstrates a relative bioavailability study that compared the exemplary mexiletine hydrochloride extended-release oral suspension 400 mg/5 mL (7.5 mL administered once per day) to the formulation with Namuscla® (mexiletine) capsule 167 mg (administered thrice in one day).

The objective of the study was to compare the relative bioavailability of two mexiletine hydrochloride extended-release oral suspension 400 mg/5 mL (7.5 mL administered once) formulation with Namuscla® (mexiletine) capsule 167 mg (administered thrice in one day) in healthy, adult, human male subjects under fasting conditions and also to monitor the safety of subjects.

The study design and plan were as follows:

Test Product (T1 and T2): mexiletine hydrochloride extended-release oral suspension 400 mg/5 mL. Dose: 1×7.5 mL of extended-release oral suspension 400 mg/5 mL, once a day dosing (0.00 hours).

Reference Product (R): Namuscla® (mexiletine) Capsule 167 mg (equivalent to 200 mg mexiletine HCl). Dose: 3×167 mg IR capsule: three times a day with an interval of 8.00 hours between each dose (0.00, 8.00, and 16.00 hours).

Study Design: An Open Label, Balanced, Randomized, Single-Dose, Three-Treatment, Three-Sequence, Three-Period Crossover Oral Relative Bioavailability Study.

Number of Subjects: Twenty-four (24) healthy, adult, human male subjects were enrolled and dosed in the study. All subjects were fasted for at least 10.00 hours prior to dosing.

Administration: The test formulation was given orally, and the reference formulation was given to the subjects over a period of 60 minutes.

Test Product: After an overnight fast of at least 10.00 hours, 7.5 mL either of Test Product 1 (T1) or Test Product 2 (T2) as per randomization was emptied into the subject's mouth via oral syringe while in a sitting position. The subject was instructed to swallow it with about 50 mL of water from 240 mL of dosing water at ambient temperature. Part of the water (3×5 mL approximately) from the same was used to carefully rinse the syringe thrice, followed by a thorough mouth check to ensure that the drug had been swallowed. The remaining part of the 240 mL of dosing water was then given to the subject, thus ensuring complete administration of the dispensed Test product in each period. The procedure was completed within 2 minutes of the actual start time of dose administration.

Reference Product: Morning (0.00 hour) dose administration: After an overnight fast of at least 10.00 hours, one capsule of Reference Product (R) was administered orally to each subject while in a sitting position with approximately 240 mL of water at ambient temperature as per randomization schedule in each period.

Evening (8.00 hour) dose administration: After a fasting of at least 2.00 hours, one capsule of Reference Product (R) was administered orally to each subject while in a sitting position with approximately 240 mL of water at ambient temperature as per the randomization schedule in each period.

Night (16.00 hour) dose administration: After a fasting of at least 2.00 hours, one capsule of Reference Product (R) was administered orally to each subject while in a sitting position with approximately 240 mL of water at ambient temperature as per the randomization schedule in each period.

### Sampling Schedule

Sampling Schedule for Subjects Who have Received Test Product:
Twenty-six (26) blood samples were collected from each subject in each period. The pre-dose (0.00 hours) blood sample of 4.0 mL was taken not more than one hour prior to dosing in each period. Further samples of 4.0 mL each were collected at 0.50 (30 min), 1.00, 2.00, 3.00, 4.00, 5.00, 6.00, 7.00, 8.00, 9.00, 10.00, 11.00, 12.00, 13.00, 14.00, 15.00, 16.00, 18.00, 20.00, 24.00, 30.00, 36.00, 48.00, 72.00 and 96.00 hours post-dose in each period.
   Sampling Schedule for Subjects Who have Received Reference Product:

Forty-eight (48) blood samples were collected from each subject in each period. The pre-dose (0.00 hours) blood sample of 4.0 mL was taken not more than one hour prior to dosing in each period. Further samples of 4.0 mL each were collected at 0.33 (20 min), 0.67 (40 min), 1.00, 1.50, 2.00, 2.50, 3.00, 3.50, 4.00, 4.50, 5.00, 6.00, 7.00, 8.00, 8.33, 8.67, 9.00, 9.50, 10.00, 10.50, 11.00, 11.50, 12.00, 12.50, 13.00, 14.00, 15.00, 16.00, 16.33, 16.67, 17.00, 17.50, 18.00, 18.50, 19.00, 19.50, 20.00, 20.50, 21.00, 22.00, 23.00, 24.00, 30.00, 36.00, 48.00, 72.00 and 96.00-hour post-dose in each period.

Pharmacokinetic Analysis: For Mexiletine, the calculated primary pharmacokinetic parameters were AUC₀₋₂₄ and secondary pharmacokinetic parameters were Cₘₐₓ, Tₘₐₓ, AUC₀₋ₜ, AUC_{0-∞}, Kₑₗ, and t_{1/2}.

Statistical Analysis: Statistical analyses were done using SAS® system for Windows version 9.4 or higher. The log-transformed pharmacokinetic parameters AUC₀₋₂₄ for Mexiletine were analyzed using ANOVA. The intra-subject CV, Power, Ratio analysis, and 90% confidence interval for the ratio of the geometric least squares mean was computed for log-transformed pharmacokinetic parameter AUC₀₋₂₄.

The % Relative Bioavailability was calculated as follows:

$$\frac{{AUC}_{0 - 24} Test}{{AUC}_{0 - 24} Reference} \times 100$$

### Conclusion:

The ratio of Test (T1) and Reference (R) product averages (least-squares means) and its 90% Confidence.

Interval derived from the analysis of log-transformed pharmacokinetic parameters were found to be 139.60% (90% CI: 132.20%-147.40%) for AUC₀₋₂₄ and 130.09% (90% CI: 120.74%-140.15%) for Cₘₐₓ.

The ratio of Test (T2) and Reference (R) product averages (least-squares means) and its 90% Confidence Interval derived from the analysis of log-transformed pharmacokinetic parameters were found to be 106.50% (90% CI: 100.77%-112.57%) for AUC₀₋₂₄ and 86.72% (90% CI: 80.39%-93.55%) for Cₘₐₓ.

The median Tₘₐₓ (hr) was found to be 6.00 hr and 8.00 hr for T1 and T2 respectively with the corresponding Tₘₐₓ range observed to be 3.00 to 12.02 hr for T1 and 5.00 to 16.00 hr for T2.

The relative bioavailability for T1 was higher than the reference product based on a comparison of Mexiletine Cₘₐₓ and AUC.

The relative bioavailability for T2 was comparable to that of the reference product based on a comparison of Mexiletine Cₘₐₓ and AUC. Test product 1 (T1), test product 2 (T2), and reference product (R) were found to be safe and well tolerated upon administration of a single dose in healthy, adult, human male subjects under fasting conditions.

A comparison of the geometric least square mean, ratios, 90% CI, and ISCV for Mexiletine HCl for Test product 1 vs. reference product, the data for which is presented in Table 28 (below).

TABLE 28
Geometric least square mean, ratios, 90% CI, and ISCV for Mexiletine HCl for Test
product 1 Vs Reference product.

| | Geometric Least Square | | T/R | | | | |
|---|---|---|---|---|---|---|---|
| | Means | | ratio | 90% CI | | ISCV | |
| Parameter | Test (T) | Reference (R) | (%) | LCL | UCL | (%) | Power |
| AUC₀₋₂₄ | 15965.7530 | 11437.1890 | 139.60 | 132.20 | 147.40 | 10.44 | 1.00 |
| (hr*ng/ml) | | | | | | | |
| Cₘₐₓ | 1085.7073 | 834.5941 | 130.09 | 120.74 | 140.15 | 14.34 | 1.00 |
| (ng/mL) | | | | | | | |
| AUC₀₋ₜ | 23759.1230 | 21493.7020 | 110.54 | 103.35 | 118.23 | 12.85 | 1.00 |
| (hr*ng/ml) | | | | | | | |
| AUC_{0-∞} | 24207.8160 | 21884.0340 | 110.62 | 103.39 | 118.35 | 12.91 | 1.00 |
| (hr*ng/ml) | | | | | | | |

As shown in Table 28 (above), the AUC₀₋₂₄ for Test product 1 is 15965.7530 hr*ng/mL when administered once per day, which is greater than that of the Reference product, which is 11437.1890 hr*ng/mL when administered thrice per day. As further shown in Table 28 (above), the Cₘₐₓ for Test product 1 is 1085.7073 ng/mL when administered once per day, which is greater than that of the Reference product, which is 834.5941 ng/mL when administered thrice per day. These results exemplify that compositions according to the invention can exhibit greater bioavailability, such as having an AUC₀₋₂₄ of about 15900 hr*ng/mL, such as about 15500-16500 hr*ng/mL, and a Cₘₐₓ of about 1080 ng/mL, such as about 1050-1150 ng/mL, when administered once-per-day when compared to the Reference product when administered thrice per day.

A comparison of the geometric least square mean, ratios, 90% CI, and ISCV for Mexiletine HCl for Test product 2 vs. reference product, the data for which is presented in Table 29 (below).

TABLE 29
Geometric least square mean, ratios, 90% CI, and ISCV for Mexiletine HCl for Test
product 2 Vs Reference product.

| | Geometric Least Square | | | | | | |
|---|---|---|---|---|---|---|---|
| | Means | | T/R | | | | |
| | | Reference | ratio | 90% CI | | ISCV | |
| Parameter | Test (T) | (R) | (%) | LCL | UCL | % | Power |
| AUC₀₋₂₄ | 12180.9070 | 11437.1890 | 106.50 | 100.77 | 112.57 | 10.44 | 1.00 |
| (hr*ng/ml) | | | | | | | |
| Cₘₐₓ | 723.7411 | 834.5941 | 86.72 | 80.39 | 93.55 | 14.34 | 1.00 |
| (ng/ml) | | | | | | | |
| AUC₀₋ₜ | 20003.1990 | 21493.7020 | 93.07 | 86.91 | 99.65 | 12.85 | 1.00 |
| (hr*ng/ml) | | | | | | | |
| AUC_{0-∞} | 20586.5830 | 21884.0340 | 94.07 | 87.82 | 100.76 | 12.91 | 1.00 |
| (hr*ng/mL) | | | | | | | |

As shown in Table 29 (above), the AUC₀₋₂₄ for Test product 2 is 12180.9070 hr*ng/mL when administered once per day, which is greater than that of the Reference product, which is 11437.1890 hr*ng/mL when administered thrice per day. As further shown in Table 29 (above), the Cₘ, for Test product 2 is 723.7411 ng/mL when administered once per day, which is greater than that of the Reference product, which is 834.5941 ng/mL when administered thrice per day. These results exemplify that compositions according to the invention can exhibit greater bioavailability, such as having an AUC₀₋₂₄ of about 12180 hr*ng/mL, such as about 12000 hr*ng/mL to about 13000 hr*ng/mL, and a Cₘₐₓ of about 720 ng/mL, such as about 700 ng/mL to about 800 ng/mL, when administered once-per-day, than the Reference product when administered thrice per day.

A summary of the PK parameters of Test Product 2 is presented in Table 30 (below).

TABLE 30
Summary of PK parameters of Test Product 2 (T2).

| | | | | | | | Geometric |
|---|---|---|---|---|---|---|---|
| Parameter | Mean | SD | Min | Median | Max | CV % | Mean |
| AUC₀₋₂₄ | 12826.7066 | 3890.5966 | 7408.731 | 12307.154 | 21997.054 | 30.33 | 12317.5205 |
| (hr*ng/mL) | | | | | | | |
| Cₘₐₓ | 763.2237 | 238.1657 | 391.667 | 762.506 | 1335.036 | 31.21 | 729.9618 |
| (ng/ml) | | | | | | | |
| AUC₀₋ₜ | 22301.7312 | 9786.0271 | 11018.334 | 20640.891 | 42422.734 | 43.88 | 20453.2331 |
| (hr*ng/ml) | | | | | | | |
| AUC_{0-∞} | 23345.7516 | 10902.0930 | 11187.093 | 20771.880 | 47308.706 | 46.70 | 21211.8309 |
| (hr*ng/mL) | | | | | | | |
| Tₘₐₓ (hr) | 8.952 | 3.542 | 5.00 | 8.00 | 16.00 | 39.57 | 8.365 |
| t_{1/2} | 13.7536 | 5.4815 | 6.821 | 12.690 | 31.553 | 39.86 | 12.9261 |
| Kel | 0.0566 | 0.0186 | 0.022 | 0.055 | 0.102 | 32.86 | 0.0536 |

As shown in Table 30 (above), the mean AUC₀₋₂₄ for Test product 2 is 12826.7066 hr*ng/mL when administered once per day. As further shown in Table 30 (above), the mean Cₘₐₓ for Test product 2 is 763.2237 ng/mL when administered once per day. These results exemplify that compositions according to the invention can exhibit greater bioavailability than the Reference product (administered three times per day), such as having a mean AUC₀₋₂₄ of about 12820 hr*ng/mL, such as about 12000 hr*ng/mL to about 13000 hr*ng/mL, and a mean Cₘₐₓ of about 760 ng/mL, such as about 700 ng/mL to about 800 ng/mL, when administered once-per-day.

A linear mean plot of Plasma concentration (ng/mL) vs. time (hours) for the Reference, Test Product 1, and Test Product 2 are presented in FIG. 1.

As shown in FIG. 1, both T1 and T2 exhibit a gradual increase in plasma concentration with a single peak in plasma concentration via once-a-day administration, compared to the Reference product (administered three times per day), which exhibits lower peaks in plasma concentration. As further shown in FIG. 1 (above), T1 exhibits a max plasma concentration of about 1000 ng/mL at about 8 hours post-administration. Additionally, as shown in FIG. 1 (above), T2 exhibits a max plasma concentration of about 700 ng/mL at about 8 hours post-administration. These results exemplify the blood plasma levels for T2 exhibit the following: (1) a max blood plasma concentration of about 700 ng/mL, such as about 650-750 ng/mL, at about 8 hours, such as at about 7 to 10 hours; (2) a stabilized decrease in blood plasma concentration post max blood plasma concentration beginning at about 8 hours post administration, such as at about 7-10 hours post administration, and lasting a period of about 8 hours, such as a period of about 7-10 hours, during which time the change in blood plasma concentration over any 1 hour of the period is less than about 2%, such as less than about 0.5-2.5%, from the subsequent hour; and (3) a drop off in blood plasma concentration, beginning at about 15 hours post administration, such as at about 12-20 hours post administration, during which time the decrease in blood plasma concentration over any 1 hour of the period is greater than 10%, such as greater than 9-15%, from the subsequent hour.

### Example 11: Food Effect Study

The objective of the study was to assess the food effect on the pharmacokinetics of mexiletine hydrochloride extended-release oral suspension 400 mg/5 mL (Dose: 7.5 mL) administered under fasting condition and under fed conditions (with standard breakfast and high fat; high-calorie nonveg breakfast) in healthy, adult, human male subjects and also to monitor the safety of the subjects.

The study design and plan were as follows:

Test Product: Mexiletine hydrochloride extended-release oral suspension 400 mg/5 mL.

Dose: 1×7.5 mL of extended-release oral suspension 400 mg/5 mL, Once a day dosing.

Study Design: An open-label, balanced, randomized, single-dose, three-treatment, three-sequence, three-period crossover oral food effect study.

Study Treatment A: Test product 2 (T2) was administered under fasting conditions.

Study Treatment B: Test product 2 (T2) was administered under fed conditions after standard breakfast (approximately 500 Kcal), which was served 30 minutes before dosing.

Study Treatment C: Test product 2 (T2) was administered under fed conditions after high fat; high-calorie nonveg breakfast (approximately 1000 calories) which was served 30 minutes before dosing.

Number of Subjects: Twenty-four (24) healthy, adult, human male subjects were enrolled and dosed in the study.

Administration for those receiving Treatment A: After an overnight fast of at least 10.00 hours, a single dose of 7.5 mL of Test Product 2 (T2) as per randomization was emptied into the subject's mouth via beaker while in a sitting position. The subject was instructed to swallow it with about 50 mL of water from approximately 240 mL of dosing water at ambient temperature. The beaker was rinsed three times with approximately 3×5 mL water and the rinse was administered to the subject, followed by a thorough mouth check to ensure that the drug had been swallowed. The procedure was completed within 2 minutes of the actual start time of dose administration. The remaining part of the 240 mL of dosing water was then given to the subject, thus ensuring complete administration of the dispensed Investigational products in each period.

Administration for those receiving Treatment B: After overnight fasting of at least 10.00 hours, a standard breakfast was served 30 minutes prior to dosing. Subjects have to consume the whole standard breakfast within 30 minutes of it being served. Exactly 30 minutes after the actual start time of a standard breakfast, a single dose of 7.5 mL of Test Product 2 (T2) as per randomization was emptied into the subject's mouth via beaker while in a sitting position. The subject was instructed to swallow it with about 50 mL of water from approximately 240 mL of dosing water at ambient temperature. The beaker was rinsed three times with approximately 3×5 mL water, and the rinse was administered to the subject, followed by a thorough mouth check to ensure that the drug had been swallowed. The procedure was completed within 2 minutes of the actual start time of dose administration. The remaining part of the 240 mL of dosing water was then given to the subject, thus ensuring complete administration of the dispensed Investigational products in each period.

Administration for those receiving Treatment C: After overnight fasting of at least 10.00 hours, a standard breakfast was served 30 minutes prior to dosing. Subjects have to consume whole standard high-fat, high-calorie, non-veg breakfast within 30 minutes of it being served. Exactly 30 minutes after the actual start time of a standard high-fat, high-calorie, non-veg breakfast, a single dose of 7.5 mL of Test Product 2 (T2) as per randomization was emptied into the subject's mouth via beaker while in a sitting position. The subject was instructed to swallow it with about 50 mL of water from approximately 240 mL of dosing water at ambient temperature. The beaker was rinsed three times with approximately 3×5 mL water and the rinse was administered to the subject, followed by a thorough mouth check to ensure that the drug had been swallowed. The procedure was completed within 2 minutes of the actual start time of dose administration. The remaining part of the 240 mL of dosing water was then given to the subject, thus ensuring complete administration of the dispensed Investigational products in each period.

Sampling Schedule: Twenty-six (26) blood samples were collected from each subject in each period.

The pre-dose (0.00 hours) blood sample of 4.0 mL was taken not more than one hour prior to dosing in each period. Further samples of 4.0 mL each were collected at 0.50 (30 min), 1.00, 2.00, 3.00, 4.00, 5.00, 6.00, 7.00, 8.00, 9.00, 10.00, 11.00, 12.00, 13.00, 14.00, 15.00, 16.00, 18.00, 20.00, 24.00, 30.00, 36.00, 48.00, 72.00, and 96.00 hours post-dose in each period.

Pharmacokinetic Analysis: Concentration vs. time profiles of Mexiletine were estimated by using Phoenix WinNonlin® version 8.3.3 or higher version of Pharsight Corporation, USA, and pharmacokinetic parameters were calculated: Primary parameters were Cₘₐₓ and AUC_{0-∞}. Secondary parameters were AUC₀₋ₜ, Tₘₐₓ, Kₑₗ, and T_{1/2}.

Statistical Analysis: Statistical analysis was done using the SAS® system for Windows version 9.4 or higher (SAS® Institute Inc., USA) The log-transformed pharmacokinetic parameters Cₘₐₓ, and AUC_{0-∞} for Mexiletine were analyzed using ANOVA. The intra-subject CV, Power, Ratio analysis, and 90% confidence interval for the ratio of the geometric least squares mean were computed for log-transformed pharmacokinetic parameters Cₘₐₓ, and AUC_{0-∞}.

Conclusion: An increase in Cₘₐₓ and AUC was observed in the Fed state (both Standard and high-calorie breakfast) compared to the Fasted state. In conclusion, food increases Cₘₐₓ (^{˜}39%) and AUC (^{˜}16%) of mexiletine hydrochloride.

Food effect studies were performed on Test Product 2 (T2), Treatment B, and Treatment A, the data for which is presented via comparison in Table 31 (below).

TABLE 31
Food effect study for Test Product 2 (T2) Comparison Treatment B Vs Treatment A.

| | Geomean | Geomean | Ratio | Lower | Upper | | |
|---|---|---|---|---|---|---|---|
| Parameter | (Treatment A) | (Treatment B) | (B/A) | Limit | Limit | ISCV | Power |
| Cₘₐₓ | 825.1137 | 1143.4828 | 138.58 | 132.42 | 145.04 | 9.25 | 1.00 |
| (ng/mL) | | | | | | | |
| AUC_{0-∞} | 19807.1980 | 23063.0820 | 116.44 | 109.29 | 124.05 | 12.90 | 1.00 |
| (hr*ng/mL) | | | | | | | |

As shown in Table 31 (above), the geomean Cₘₐₓ for Treatment A is 825.1137 ng/mL and the geomean AUC_{0-∞} is 19807.1980 hr*ng/mL under fasting conditions. As further shown in Table 31, the geomean Cₘₐₓ for Treatment B is 1143.4828 ng/mL and the geomean AUC_{0-∞} is 23063.0820 hr*ng/mL under conditions including a standard breakfast. These results exemplify that compositions according to the invention can exhibit greater bioavailability, when administered with food rather than under fasting conditions, such as having a geomean Cₘₐₓ of about 1140 ng/mL, such as about 1100-1200 ng/mL, and the geomean AUC_{0-∞} of about 23000 hr*ng/mL, such as about 22500-23500 hr*ng/mL.

Food effect studies were performed on Test Product 2 (T2), Treatment C, and Treatment A, the data for which is presented via comparison in Table 32 (below).

TABLE 32
Food effect study for Test product 2 (2) comparison Treatment C Vs Treatment A.

| | Geomean | Geomean | Ratio | Lower | Upper | | |
|---|---|---|---|---|---|---|---|
| Parameter | (Treatment A) | (Treatment C) | (C/A) | Limit | Limit | ISCV | Power |
| Cₘₐₓ | 825.1137 | 1036.8176 | 125.66 | 120.15 | 131.41 | 9.25 | 1.00 |
| (ng/mL) | | | | | | | |
| AUC_{0-∞} | 19807.1980 | 22382.1160 | 113.00 | 106.17 | 120.27 | 12.90 | 1.00 |
| (hr*ng/mL) | | | | | | | |

As shown in Table 32 (above), the geomean Cₘₐₓ for Treatment A is 825.1137 ng/mL and the geomean AUC_{0-∞} is 19807.1980 hr*ng/mL under fasting conditions. As further shown in Table 32, the geomean Cₘₐₓ for Treatment C is 1036.8176 ng/mL and the geomean AUC_{0-∞} is 22382.1160 hr*ng/mL under conditions including a whole standard high-fat high-calorie non-veg breakfast. These results exemplify that compositions, according to the invention can exhibit greater bioavailability, when administered with a whole standard high-fat high-calorie non-veg breakfast rather than under fasting conditions, such as having a geomean Cₘₐₓ of about 1030 ng/mL, such as about 1000-1100 ng/mL, and the geomean AUC_{0-∞} of about 22300 hr*ng/mL, such as about 22500-23500 hr*ng/mL.

A linear plot of plasma concentration (ng/mL) vs. time (hours) of the data collected from the food effect studies for Compositions A, B, and C are presented in FIG. 2.

As shown in FIG. 2, all three compositions exhibit a gradual increase in plasma concentration with a single peak in plasma concentration, via once-a-day administration. As further shown in FIG. 2, Composition B, administered under conditions including a standard breakfast, exhibits the highest maximum plasma concentration of about 1200 ng/mL, such as about 1150-1300 ng/mL, at about 6 hours post-administration, such as at about 5-7 hours post-administration. As further shown in FIG. 2, Composition C, administered under conditions including a high-fat high-calorie non-veg breakfast, exhibits the second highest maximum plasma concentration of about 1000 ng/mL, such as about 950-1050 ng/mL, at about 6 hours post-administration, such as at about 5-7 hours post-administration. Lastly, as shown in FIG. 2, Composition A, administered under fasting conditions, exhibits the lowest maximum plasma concentration of about 750 ng/mL, such as about 700-800 ng/mL, at about 6 hours post-administration, such as at about 5-7 hours post-administration.

As further exemplified by the results of this study, and those above, this invention provides various forms and methods for making compositions that may likely be advantageous to the treatment population, especially under conditions including administration post a standard breakfast.

What is claimed is:

1. A pharmaceutical composition for oral administration comprising (a) a therapeutically effective amount of mexiletine or pharmaceutically acceptable salt thereof, (b) an effective amount of a pharmaceutically acceptable ion exchange resin, and (c) one or more pharmaceutically acceptable excipients, wherein the mexiletine or pharmaceutically acceptable salt thereof is released in a controlled release manner when the composition is administered to a human subject such that once-daily administration of the composition is effective for the treatment of myotonia.

2. The composition of claim 1, wherein the composition comprises about 100-about 600 mg of mexiletine hydrochloride.

3. The composition of claim 2, wherein the composition comprises about 200-about 600 mg of mexiletine hydrochloride.

4. The composition of claim 1, wherein the composition exhibits a median, Tₘₐₓ of about 5 to about 8 hours, the composition exhibits a mean t_{1/2} of about 10 hours to about 13.5 hours, or the composition exhibits both a Tₘₐₓ of about 5 to about 8 hours and a mean t_{1/2} of about 10 hours to about 13.5 hours.

5. The composition of claim 4, wherein the composition further exhibits:
(a) a mean Cₘₐₓ of about 700-1250 ng/mL;
(b) a mean AUC₀₋₂₄ of about 12000 ng*hr/mL to about 17500 ng*hr/mL;
(c) a mean AUC₀₋ₜ of about 20000-24500 ng*hr/mL;
(d) a mean AUC_{0-∞} of about 20000-26000 ng*hr/mL; or
(e) any combination of any or all of (a)-(d)
after single-dose administration.

6. The composition of claim 1, wherein the composition exhibits at least two of:
(a) a mean Tₘₐₓ of about 5 to about 8 hours;
(b) a mean Cₘₐₓ of about 700-1250 ng/mL;
(c) a mean AUC₀₋₂₄ of about 12000 ng*hr/mL to about 17500 ng*hr/mL;
(d) a mean AUC₀₋ₜ of about 20000-24500 ng*hr/mL;
(e) a mean AUC_{0-∞} of about 20000-26000 ng*hr/mL; and
(f) a mean t_{1/2} of about 10 hours to about 13.5 hours
after single-dose administration.

7. The composition of claim 1, wherein the pharmaceutical composition exhibits a statistically significant increase in mean Cₘₐₓ, mean AUC, or both, in a fed condition compared to a fasting condition.

8. The composition of claim 4, wherein the pharmaceutical composition exhibits a statistically significant increase in mean Cₘₐₓ, mean AUC, or both, in a fed condition compared to a fasting condition.

9. The composition of claim 1, wherein the composition exhibits an in vitro dissolution profile characterized by
(a) (1) after 0-2 hours, from about 0% to about 30% by weight of mexiletine is released from the composition, after 0-4 hours from about 10% to about 40% by weight of mexiletine is released from the composition, (2) after 4-12 hours from about 30% to about 70% by weight of mexiletine is released from the composition, (3) from 6-14 hours more than about 60% by weight of mexiletine is released from the composition, and (4) more than about 80% by weight of mexiletine is released from the composition within about 24 hours, when measured using a USP type IV apparatus having flow rate 8 ml/min along with 6 gm glass beads in 0.1N HCl media, 37° C.±0.5° C.;
(b) (1) after 0-2 hours from about 0% to about 25% by weight of mexiletine is released from the composition, (2) after 0-4 hours from about 10% to about 40% by weight of mexiletine is released from the composition, and (3) after 4-12 hours from about 30% to about 100% of the mexiletine is released from the composition within about 24 hours when measured using a USP type IV apparatus having flow rate 8 ml/min along with 6 gm glass beads in pH 5.0 acetate buffer media, 37° C.±0.5° C.;
(c) (1) after 0-2 hours from about 0% to about 35% by weight of mexiletine is released from the composition, (2) after 0-4 hours from about 10% to about 50% by weight of mexiletine is released from the composition, (3) after 4-12 hours from about 30% to about 100% from the composition, when measured using a USP type IV apparatus having flow rate 8 ml/min along with 6 gm glass beads in pH 6.8 phosphate buffer media, 37° C.±0.5° C.;
(d) the composition (1) releases not more than 50% by weight of mexiletine in an initial 1 hour in 500 mL 0.1N HCl and (2) followed by 700 mL pH 4.5 acetate buffer changeover media the said composition releases not less than 65% by weight of mexiletine in 2 hours and (3) further followed by 900 mL pH 6.8 phosphate buffer changeover media the composition releases not less than 85% by weight of mexiletine in 4 hours when measured in a United States Pharmacopoeia (USP) type II dissolution apparatus, rotated at 75 rpm at a temperature of 370±0.5° C.; or
(e) a combination of any or all of (a)-(d).

10. The composition of claim 4, wherein upon a single oral administration of the composition (1) Tₘₐₓ occurs between about 5 to about 8 hours after administration in most human subjects and (2) the plasma concentration of mexiletine in most human subjects after 16 hours administration is within at least about 33% of Cₘₐₓ.

11. The composition of claim 4, wherein the plasma concentration in most human patients receiving a single oral administration of the composition remains within about 20% of Cₘₐₓ for a period of at least about 4 hours.

12. The composition of claim 4, wherein the plasma concentration of mexiletine in most human patients receiving a single oral administration of the compound at either 3 hours after administration, 4 hours after administration, or both, is within about 25% of the plasma concentration of mexiletine 18 hours after administration, 20 hours after administration, or both.

13. The composition of claim 1, wherein more than 50% of the active pharmaceutical ingredient content of the composition is composed of mexiletine or pharmaceutically acceptable salt thereof.

14. The composition of claim 13, wherein the only active pharmaceutical ingredient in the composition consists of mexiletine or pharmaceutically acceptable salt thereof.

15. The composition of claim 14, wherein the pharmaceutical composition exhibits an increase in mean Cₘₐₓ, mean AUC, or both, in a fed condition with respect to fasting condition.

16. The composition of claim 13, wherein the composition exhibits a mean t_{1/2} of about 10 hours to about 13.5 hours, a median Tₘₐₓ of about 5 to about 8 hours, or both.

17. The composition of claim 16, wherein the ratio of mexiletine or pharmaceutically acceptable salt thereof to the pharmaceutically acceptable ion exchange resin in the composition is about 1:0.5 to about 1:5.

18. The composition of claim 16, wherein the mexiletine or pharmaceutically acceptable salt thereof and the ion exchange resin form a pharmaceutically acceptable mexiletine resin complex wherein, the ion exchange resin is about 30% to about 85% of the mexiletine-resin complex on a weight-to-weight basis.

19. The pharmaceutical composition of claim 16, wherein the composition further comprises (c) one or more suitable release retarding agents selected from a hydrophilic release retardant polymer, a hydrophobic release retardant polymer, or both.

20. The composition of claim 16, wherein the composition further comprises a suspension base selected from starch or disaccharide or both.

21. A pharmaceutical composition for oral administration comprising a therapeutically effective amount of mexiletine or pharmaceutically acceptable salt thereof and an effective amount of at least one release retarding agents, wherein the composition exhibits:
(a) a median Tₘₐₓ about 5 to about 8 hours;
(b) a mean Cₘₐₓ about 700-1250 ng/mL;
(c) a mean AUC₀₋₂₄ about 12000 ng*hr/mL to about 17500 ng*hr/mL;
(d) a mean AUC₀₋ₜ of about 20000-24500 ng*hr/mL;
(e) a mean AUC_{0-∞} about 20000-26000 ng*hr/mL;
(f) a mean t_{1/2} about 10 hours to about 13.5 hours; or
(g) any combination of any or all of (a)-(f)
after single-dose administration.

22. The composition of claim 21, wherein more than 50% of the active pharmaceutical ingredient content of the composition is composed of mexiletine or pharmaceutically acceptable salt thereof.

23. The composition of claim 22, wherein the only active pharmaceutical ingredient in the composition consists of mexiletine or pharmaceutically acceptable salt thereof.

24. A pharmaceutical composition for oral administration, the pharmaceutical composition being formulated in a pharmaceutically suitable liquid oral dosage form and comprising (1) a therapeutically effective amount of mexiletine or pharmaceutically acceptable salt thereof and (2) an amount of at least one pharmaceutically acceptable release retarding agent that following administration of the composition to a human subject releases the mexiletine or pharmaceutically acceptable salt thereof in a controlled release manner such that once-daily administration of the composition is effective for the treatment of myotonia.

25. The composition of claim 24, wherein at least most of the active pharmaceutical ingredient content of the composition is composed of mexiletine or pharmaceutically acceptable salt thereof.

26. The composition of claim 25, wherein the only active pharmaceutical ingredient in the composition consists of mexiletine or pharmaceutically acceptable salt thereof.

27. A method of treating myotonia in a human subject in need thereof comprising administering an effective amount of a composition according to claim 1 to the human subject.

28. The method of claim 27, wherein the method comprises administering the composition to the subject only once per day.

29. The method of claim 28, wherein the method comprises administering the effective amount of the composition to the subject with food once per day.

30. A method of treating myotonia in a human subject in need thereof comprising administering an effective amount of a composition according to claim 21 to the subject.

31. The method of claim 30, wherein the method comprises administering the composition to the subject only once per day.

32. The method of claim 31, wherein the method comprises administering the effective amount of the composition to the subject with food once per day.

33. A method of treating myotonia in a human subject in need thereof comprising administering an effective amount of a composition according to claim 24 to the subject.

34. The method of claim 33, wherein the method comprises administering the composition to the subject only once per day.

35. The method of claim 34, wherein the method comprises administering the effective amount of the composition to the subject with food once per day.
